(19) United States
(12) Patent Application Publication
(10) Pub. No.: US 2026/0275378 A1
(43) Pub. Date: Sep. 17, 2026

(54) AAV VARIANTS FOR TREATMENT OF COMPLEMENT DISORDERS

(71) Applicant: 4D MOLECULAR THERAPEUTICS INC., Emeryville, CA (US)

(72) Inventors: Melissa Calton, Emeryville, CA (US); Roxanne CROZE, Emeryville, CA (US); Albena KANTARDZHIEVA, Emeryville, CA (US)

(73) Assignee: 4D MOLECULAR THERAPEUTICS INC., Emeryville, CA (US)

(21) Appl. No.: 19/473,440

(22) PCT Filed: Apr. 3, 2024
(86) PCT No.: PCT/US2024/022843

§ 371 Date: Oct. 7, 2025

Related U.S. Application Data
(60) Provisional application No. 63/494,925, filed on Apr. 7, 2023.
(60) Provisional application No. 63/586,227, filed on Sep. 28, 2023.

(51) Int. Cl. C12N 15/86; C07K 14/005; C07K 14/47

(52) U.S. Cl. CPC C12N 15/86; C07K 14/005; C07K 14/472; C12N 2750/14122; C12N 2750/14143; C12N 2830/48; C12N 2830/50

(57) ABSTRACT

Recombinant AAV (rAAV) comprising a variant adeno-associated virus (AAV) capsid and a transgene encoding a human factor H variant are provided. Also provided are methods of delivering the transgene to the retina and methods of treating dry age-related macular degeneration and geographic atrophy secondary to age-related macular degeneration disorders by contacting retinal cells with the rAAV.

### CROSS REFERENCE TO RELATED APPLICATIONS

This application claims the benefit of U.S. Provisional Patent Application Serial Nos. 63/494,925, filed Apr. 7, 2023 and 63/586,227, filed Sep. 28, 2023, the entire contents of each of which are incorporated herein by reference.

### SEQUENCE LISTING SUBMISSION VIA EFS-WEB

A computer readable XML file, entitled “090400-5022-WO-Sequence-Listing” created on Apr. 2, 2024, with a file size of about 87,900 bytes contains the sequence listing for this application and is hereby incorporated by reference in its entirety.

### BACKGROUND OF THE INVENTION

A number of human diseases are caused by complement dysregulation, resulting in complement-mediated autologous tissue injury. The complement dysregulation may arise from mutations, either somatic or germline, in complement regulator or regulator-related genes such that these regulators no longer function normally. In particular, there are common and rare human diseases that are caused by excessive complement activation resulting from dysregulation of the complement activation cascade.

Current therapeutic approaches are focused on the development of reagents such as monoclonal antibodies (mAbs), peptides or other small molecules that bind and block specific alternative pathway or terminal pathway complement components, A clinically validated example is Eculizamab, a humanized mAb against complement C5 which has been approved for the treatment of paroxysmal nocturnal hemoglobinuria (PNH) and atypical hemolytic uremic syndrome (aHUS). Other approaches that have been described include mAbs against factor B (fB), factor D (fD), or properdin (fP), and a cyclic peptide that binds and inhibits C3. A limitation of these approaches is that they require repeated and inconvenient intravenous (IV) dosing of patients. Further, since these treatments block the alternative pathway or terminal pathway, they run the risk of compromising host defense. Indeed, patients on Eculizumab therapy have to be vaccinated against bacteria strains that cause lethal meningitis and these patients are also put on prophylactic antibiotic therapy before being treated with the approved mAb drug.

In other approaches, recombinant regulatory proteins such as soluble DAF, CR1, CRIg and proteins comprising minimal domains of fluid phase regulator fH (N-terminal short consensus repeat [SCR]1-5 and C-terminal SCR 19-20) or fusion proteins between fHT and CR2 (TT30) have been tested. However, large scale heterologous expression of such proteins as therapeutic drugs requires significant effort, and animal studies have shown their in vivo clearance rate after administration to be fast making such therapeutic strategies cumbersome and less practical as multiple and frequent administrations of such protein drugs would be required.

A need remains in the art for compositions useful for treating complement-mediated diseases with greater and longer-lasting efficacy.

### SUMMARY OF THE INVENTION

Described herein are recombinant AAV (rAAV) virions comprising a variant AAV capsid sequence encapsulating a heterologous nucleic acid comprising an engineered human complement regulator factor H (f) gene operably linked to an expression control sequence, wherein the human fH (hfH) gene encodes a soluble MH protein variant that retains complement regulatory function, wherein said fH variant comprises short consensus repeats (SCRs) 1, 2, 3, 4, 19 and 20. In some embodiments, the rAAV comprises a heterologous nucleic acid comprises an hfH gene encoding a soluble hfH protein variant that retains complement regulatory function, wherein said fH variant comprises or consists of or consists essentially of SCRs, wherein the SCRs are selected from the group consisting of SCR1, SCR2, SCR3, SCR4, SCR6, SCR7, SCR8, SCR17, SCR18, SCRSCR19 and SCR20.

In related aspects, an rAAV virion is provided comprising a variant AAV capsid sequence encapsulating a heterologous nucleic acid, wherein the nucleic acid comprises nucleotide sequence encoding an engineered hfH variant comprising a leader sequence and human complement receptor SCRs, wherein the SCRs are selected from the group consisting of: (a) SCR1-4, 7, and 19-20; (b) SCR1-4, 6, 7, and 19-20; (c) SCR1-4, 7, 8, and 19-20; (d) SCR1-4, 6, 7, 8, and 19-20; (e) SCR1-4, 17, and 19-20; (f) SCR1-4, and 18-20; (g) SCR1-4, and 17-20; (h) SCR1-4, 7, and 18-20; (i) SCR1-4, 6, 7, and 18-20; (j) SCR11-4, 7, 8 and 18-20; (k) SCR1-4, 6, 7, 8 and 18-20; (1) SCR1-4, 7, and 17-20; (m) SCR1-4, 6, 7, and 17-20; (n) SCR1-4, 7, 8 and 17-20; or (o) SCR1-4, 6, 7, 8 and 17-20. Optionally, at least one glycosylation site is engineered into at least one of the SCRs.

The variant AAV capsid protein of the rAAV comprises a capsid protein comprising a peptide insertion of from about 7 amino acids to about 20 amino acids (a “heterologous peptide” or “peptide insertion”) in the GH-loop of the capsid protein, preferably in a surface-exposed region of the Gil-loop, relative to a corresponding parental AAV capsid protein, wherein the peptide insertion comprises the amino acid sequence ISDQTKH (SEQ 1) NO;1). In some preferred aspects, the peptide insertion has from 1 to 3 amino acid spacer amino acids (Y₁-Y₃) at the amino and/or carboxyl terminus of the amino acids sequence TSDQTKH (SEQ ID NO:1), wherein each of Y₁-Y₃ is independently selected from Ala, Leu, Gly, Ser, Thr, and Pro. In some embodiments, the peptide insertion comprises 2 spacer amino acids at the N-terminus and 1 spacer amino acids at the C-terminus. In particularly preferred embodiments, the peptide insertion comprises, consists essentially of or consists of the amino acid sequence LAISDQTKHA (SEQ ID NO:2). In certain preferred embodiments, the insertion site is between amino acids 587 and 588 of VP1 of AAV2 or is between amino acids 588 and 589 of AAV2 or the corresponding positions in the capsid protein of another AAV serotype. In some embodiments, the capsid protein further comprises one or more amino acid substitutions relative to VP1 capsid of AAV2 or one or more corresponding substitutions in another AAV serotype, preferably wherein the capsid protein further comprises a P34A amino acid substitution relative to VP1 capsid of AAV2 or the corresponding substitution in another AAV serotype.

In other embodiments, a method for delivering a heterologous nucleic acid comprising a nucleotide sequence encoding a soluble hfH protein variant as herein described to a mammalian subject is provided, the method comprising administering to the mammal an effective amount of an rAAV as herein described or a pharmaceutical composition comprising same, preferably wherein the rAAV or pharmaceutical composition is administered by intravitreal injection. In some aspects, the heterologous nucleic acid is delivered to a retinal cell of the subject, e.g., a photoreceptor cell (e.g., rods; cones), a retinal ganglion cell (ROC), a glial cell (e.g., a Müller glial cell, a microglial cell), a bipolar cell, an amacrine cell, a horizontal cell, and/or a retinal pigmented epithelium (RPE) cell of the subject.

In some embodiments, detectable plasma levels of the hfH variant are present in a subject for at least a week, at least two weeks, at least three weeks, at least a month, at least two months, or at least 6 months following administration of the rAAV to the subject. In particularly preferred embodiments, the rAAV is administered to a subject by intravitreal administration.

In other embodiments, a pharmaceutical composition is provided comprising an rAAV as described herein and a pharmaceutically acceptable excipient.

In other aspects, a method is provided for treating a complement related disorder by delivering to the subject an rAAV as herein described or a pharmaceutical composition comprising the rAAV. Complement related disorders that may be treated include, without limitation, membranoproliferative glomerulonephritis, atypical hemolytic uremic syndrome (aHUS), age related macular degeneration (AMD), geographic atrophy secondary to AMD microangiopathic haemolytic anemia, thrombocytopenia, acute renal failure, paroxysmal nocturnal hemoglobinuria (PNH), schizophrenia, ischemic stroke, and/or bacterial infections caused by recruitment of bacterial pathogens.

In a further aspect, a method is provided for treating dry age-related macular degeneration (AMD) (e.g., late-stage dry AMD) in a subject in need thereof by delivering to the subject an effective amount of an rAAV as herein described or a pharmaceutical composition comprising the rAAV. In related aspects, the rAAV or pharmaceutical composition is administered to a subject for the treatment of geographic atrophy secondary to AMD. Preferably, the rAAV or pharmaceutical composition is administered to the subject by intravitreal injection.

### DESCRIPTION OF THE DRAWINGS

FIG. 1 is a schematic of a transgene cassette comprising 5′ and 3′ AAV invented terminal repeats (ITRs) from the AAV2 genome, a polyadenylation signal (SV40 late polyA) and nucleotide sequence encoding a codon-optimized human fH variant containing SCRs 1-4, 6-8 and 17-20 (“miniCFH”), operably linked to a CAG promoter.

FIGS. 2A-F. FIGS. 2A-F illustrate miniCFM expression, activity and function in human cells following transfection with an AAV plasmid comprising the transgene cassette illustrated at FIG. 1. FIGS. 2A-2B illustrate dose-dependent miniCFI and full, length CFH expression in protein lysates from supematants of plasmid transfected HEK293T cells by ELISA (FIG. 2A) and western blot (FIG. 2B). FIG. 2C illustrates C3b binding: preincubation with blocking CFH antibodies, but not with control antibodies or mock incubation, reduces CFH and miniCFH signal, indicating that miniCFH binds directly to C3b.

FIG. 2D illustrates heparin binding: preincubation with blocking CFH antibodies reduces recombinant CFH and miniCFH signal, indicating that miniCFH1 bind retain heparin binding activity. FIG. 2E shows the results of a C3b cleavage assay that was performed to confirm the functional activity of miniCFH expressed in HEK293T cells. The C3 protein is cleaved into C3a and C3b fragments. C3b fragment can form C3 convertase upon binding factor B or can be degraded by complement factor I (CR), in the presence of CFH as a co-factor, into smaller inactive fragments causing interruption in the alternative complement cascade. These smaller fragments resulting from the C3b degradation are observed by Western blot. FIG. 2F illustrates complement inhibition (Wieslab® Complement system Alternative pathway assay), demonstrating that supernatant from AAV plasmid cassette-transfected cells resulted in membrane attack complex (MAC) formation inhibition similar to full length CFH and anti-C5 antibody controls. NT=not transfected

FIGS. 3A-C illustrate expression, activity, and function of miniCHF in human RPE Cells. IPSC-derived retinal pigment epithelial (RPE) cells were transduced with an rAAV comprising (i) a variant AAV capsid protein comprising the amino acid sequence of SEQ ID NO:42 and (ii) a nucleic acid comprising the expression cassette illustrated at FIG. 1. RPE were transduced at different multiplicity of infection (MOI; vector genomes (vg) per cell). 4 days later, 7 days post-transduction, media was harvested and assayed for secreted miniCFH expression by ELISA (FIG. 3A) and complement inhibition activity by the Wieslab® Complement system Alternative pathway assay (FIG. 3B). Complement inhibition was also assessed by ICC (FIG. 3C). Alternative complement was activated in iPSC-derived RPE on day 6 post-transduction by adding to the culture media NIS at final concentration [1%] and Zymosan at final concentration [0.5 mg/ml of NHS]. 24 hours later, 7 days post-transduction, cells were fixed and stained with primary MAC antibody IgG2aKappa mouse anti-human (Abcam cat #59835) or Isotype control mouse IgG2aKappa (Invitrogen cat #14-4724-82) and stained with secondary A488 goat anti-mouse (Invitrogen cat #A11001) and DRAQ5 (ThermoFisher cat #62251) for nuclei. Images were taken using a fluorescence microscope. miniCFI expressed by cells at all MOIs prevented MAC formation in the cultures. An anti-CS antibody was used as a positive control for complement inhibition and prevention of MAC formation. NT=not transduced

FIG. 4 illustrates LCMS quantification of short CFH (sCFH) concentrations in aqueous humor (AH) samples of NHPs following intravitreal (bilateral) administration of the specified dose of rAAV comprising (i) a variant AAV capsid protein comprising the amino acid sequence of SEQ ID NO:42 and (ii) a nucleic acid comprising the expression cassette illustrated at FIG. 1 to the NHPs.

FIG. 5 illustrates sCFH RNA expression (by in situ hybridization analysis) in untransfected HEK 293T cells and HEK 293T cells following transfection with rAAV comprising (i) a variant AAV capsid protein comprising the amino acid sequence of SEQ ID NO:42 and (ii) a nucleic acid comprising the expression cassette illustrated at FIG. 1.

FIG. 6 illustrates the results of in situ hybridization analysis of ocular tissue from non-human primates following intravitreal (bilateral) administration of the specified dose of rAAV comprising (i) a variant AAV capsid protein comprising the amino acid sequence of SEQ ID NO:42 and (ii) a nucleic acid comprising the expression cassette illustrated at FIG. 1.

FIGS. 7A-F FIG. 7A is a Western blot that shows detection of CFH protein following transfection with CFH or miniCFH constructs. NT cell supernatant analyzed showed no band as a negative control. Recombinant full length CFH (250 kDa, Lane 5) served as a positive control. Arrows show the theoretical MW of miniCFPH (80 kDa), and full length CFH (139 kDa). Ladder is represented on the left. FIG. 7B shows CPH protein concentrations in supematants from HEK293T cells transfected with 0.125 or 0.5 jig of CFH or miniCFH construct as determined with a human Factor H ELISA, ****p<0.0001 compared to NT; \hat{ }p-0.0003 compared between 0.125 μg DNA vs 0.5 jig DNA samples. FIG. 7C shows supernatants from miniCFH DNA (0.5 μg DNA/well, 15 μl supernatant) transfected HEK cells that led to complement inhibition comparable to positive control eculizumab (6 μg), **p=0.0028, ***p=0.002 compared to NT. FIG. 7D is a Western blot of 6 reactions including various components identified on the legend at the top of the image. Purified recombinant protein components (purified CHF, CFI, C3b) and supernatants following transfection (CFH supernatant) are shown. Cleavage products are highlighted with arrows. Upon addition of supernatant following transfection of miniCFH to reaction, increased cleavage products were observed (Lane 2) compared to NT supernatants (Lane 3). Positive control reaction with all three recombinant proteins (CFH, CFI, C3b) in Lane 4 and negative controls in Lane 5-7. FIG. 7E shows miniCFH in supernatants following transfection bound to C3b and was ablated following a CFH antibody incubation, *p=0.05 to CFH antibodies **p=0.01 to CFH antibodies. FIG. 7F shows miniCFH in supernatants following transfection bound to heparin and ablated following a CFH antibody incubation, *p=0.05 to CFH antibodies **p=0.01 to CFH antibodies. NT=non-transfected Error bars±standard deviation; n=3 experimental replicates; One-way ANOVA, Tukey's post-hoc

FIG. 8 illustrates transduction of iPSC-RPE cells resulting in expression of miniCFH protein. CFH concentration [nM] in supernatant from RPE cells transduced with rAAV (comprising a capsid protein comprising the amino acid sequence set forth in SEQ ID NO:42 and a heterologous nucleic acid encoding a shortened from of complement factor H (“miniCFH”) having the amino acid sequence set forth in SEQ ID NO:34) at different MOI, which showed a dose response in CFH expression. Any signal beyond that seen from NT cells would then correlate to miniCFH expressed from the rAAV. MOI=multiplicity of infection; NT=non-transduced. Error bars±standard deviation; n=4 experiment replicates. Significance relative to NT, **p=0.0071 and ***p=0.0007. Significance \hat{ }p=0.0477 for comparison between MOI 5,000 and MOT 50,000. Statistics; one-way ANOVA, Tukey's post-hoc.

FIGS. 9A-9B illustrate inhibition of alternative complement pathway by miniCFH Expressed by iPSC-derived RPE cells transduced by rAAV comprising a capsid protein comprising the amino acid sequence set forth in SEQ ID NO:42 and a heterologous nucleic acid encoding the amino acid sequence set forth in SEQ ID NO:34, operably linked to a CAG promoter. Cell supernatants from RPE transduced with the rAAV were assayed for complement inhibiting activity by two methods: Wieslab assay (FIG. 9A) and soluble C5b-9 ELISA (FIG. 9B). FIG. 9A shows inhibition of alternative complement activity resulting from addition of the specified samples to a Wieslab assay with normal human serum. Activity readout with input of NT supernatants is defined as 0% inhibition. A dose response was observed, with higher MOI doses of the rAAV resulting in increased complement inhibition. Error bars±standard deviation; n=3 experiment replicates. Significance relative to NT unless otherwise noted. *p 0.0342, **p 0.0026, ****p<0.0001, \hat{ }\hat{ }p=0.0078 for comparison between MOI 5,000 and MOI 50,000. FIG. 9B shows soluble C5b-9 concentration in samples with supernatant from RPE transduced with the rAAV at different MOI showed an inverted dose response. Concentration of soluble C5b-9 was normalized, with C5b-9 concentration in NT supernatant 32 1. Error bars±standard deviation; n=4 experiment replicates. Significance relative to NT, **p=0.0071 and ****p<0.0001. Significance \hat{ }p=0.0272 for comparison between MOI 5,000 and MOI 50,000. Statistics; one-way ANOVA, Tukey's post-hoc. NT, non-transduced; MOT, multiplicity of infection.

FIG. 10 shows acceleration of C3 convertase decay activity by expressed miniCFH. C3b cleavage products in samples of purified C3b incubated with purified CFI and supernatant samples or purified full length CFH were detected by western blot. Samples lacking both CFI and CFH do not show cleavage of the C3b a chain, while samples with one or the other of purified CF or CFH show low levels—of cleavage products. Incubating C3b with both CFI and miniCFH results in extensive cleavage, with a marked reduction in the a chain and increased abundance of cleavage products. miniCFH from cell supematants in place of purified CFH recapitulates these effects. NT, non-transduced; MOI, multiplicity of infection. Representative blot, n=4 experiment replicates.

FIGS. HA-11B show that transduction of RPE with rAAV (comprising a capsid protein comprising the amino acid sequence set forth in SEQ ID NO:42 and a heterologous nucleic acid encoding the amino acid sequence set forth in SEQ TID NO:34, operably linked to a CAG promoter) prior to induction of complement activation in cultures protected RPE from MAC deposition. FIG. 11A shows immunocytochemistry (ICC) analysis. Representative images, n=4 experimental replicates. Scale bar=200 μm. FIG. 11B shows flow cytometry analysis. The percentage of total RPE cells with MAC deposition at different MOI showed a dose response. Error bars standard deviation; n-4 experiment replicates. Significance relative to NT, ****p<0.0001. Statistics; one-way ANOVA, Tukey's post-hoc. NT, non-transduced; MOI, multiplicity of infection. MAC=Membrane Attack Complex.

FIG. 12 shows alternative Complement inhibitory activity of rAAV (comprising a capsid protein comprising the amino acid sequence set forth in SEQ ID NO:42 and a heterologous nucleic acid encoding the amino acid sequence set forth in SEQ ID NO:34, operably linked to a CAG promoter) in an in vitro RPE disease model. iC3b concentration in a disease model supernatant from RPE transduced with the rAAV showed a decrease in comparison to NT. Concentration of iC3b was normalized, with iC3b concentration in NT supernatant 1. NT, non-transduced; MOI, multiplicity of infection; RPE, retinal pigment epithelial cells. Error bars ±standard deviation; n-4 experiment replicates. Significance relative to NT, **p=0.0021 and ****p<0.0001. Statistics; one-way ANOVA, Tukey's post-hoc.

FIG. 13 shows miniCFH concentrations in aqueous humor of nonhuman primates following intravitreal dosing with an rAAV comprising a capsid protein comprising the amino acid sequence set forth in SEQ ID NO: 42 and a heterologous nucleic acid encoding the amino acid sequence set forth in SEQ ID NO:34, operably linked to a CAG promoter. Expression of miniCFH protein in aqueous humor was measured by LC-MS. Fluid expression is shown as ng/mL, Data is summarized from 3-4 animals. Mean+standard deviation

FIG. 14 shows miniCFH. concentrations in retinal tissue and RPE/Choroid in nonhuman primates following intravitreal dosing with an rAAV comprising a capsid protein comprising the amino acid sequence set forth in SEQ ID NO:42 and a heterologous nucleic acid encoding the amino acid sequence set forth in SEQ ID NO:34, operably linked to a CAG promoter. Expression of miniCFH protein in aqueous humor (AH), retina tissue (Ret), and RPE/choroid tissue (RPE/C) from NIP eyes after IVT of the rAAV at (left) 5×10¹¹ vg/eye and (right) 1×10¹⁰ vg/eye measured by LC-MS at terminal necropsy timepoints. Data is summarized from 3-4 animals. Fluid expression shown as ng/mL, Tissue expression shown as ng/g. Mean+standard deviation.

### DETAILED DESCRIPTION OF THE INVENTION

### Definitions

As used herein, the term “functional fH variant” includes fH variants which are characterized by having complement regulating activity (cofactor activity) located in SCR1-4 and optionally, a functional C3b-binding and GAG-binding ability (located within wild-type SCR7 and SCR19-20) characteristic of wild-type fH. In some embodiments, the engineered fH variants have more than 100% of wild-type fH cofactor activity and/or GAG-binding ability. In another embodiment, the engineered fH variant has less than about 95% to about 100% of wild-type functional fH. For example, an engineered fH variant may have at least 50% of the cofactor activity present in functional wild-type fH, and more desirably at least about 60%, at least about 75%, at least about 80%, at least about 85%, at least about 90%, at least about 95%, or at least about 99%. In another embodiment, the engineered fH variant may alternatively or additionally have at least at least 50% of the GAG-binding ability of functional fH, and more desirably at least about 60%, at least about 75%, at least about 80%, at least about 85%, at least about 90%, at least about 95%, or at least about 99%. Methods of determining cofactor activity, binding and/or determining increased circulating half-life as compared to the hfH proteins are known in the art

As used herein, when reference is made to SCR #-#, the domains are inclusive of the endpoints and is the same as “SCR #, . . . SCR ##”. In certain embodiments, periods are used between the domains. For example, SCR1-4, refers to “SCR1, SCR2, SCR3, and SCR4” and is the same as “SCR1,2,3,4” or “SCR1 0.2.3.4.”. SCR19-20, refers to SCR19 and SCR20 and is the same as “SCR19,20”. For example, “SCR6-8”, “SCR6.7.8” and “SCR6,7,8” refer to the same domains.

The term “isolated” designates a biological material (cell, nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.”

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then that a single vector can contain just a single coding region, or comprise two or more coding regions.

As used herein, the term “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites and stem-loop structures. Ifa coding region is intended for expression in a eukaryotic cell, a polyadenylation signal and transcription termination sequence will usually be located 3′ to the coding sequence.

As used herein, the term “nucleic acid” is interchangeable with “polynucleotide” or “nucleic acid molecule” and a polymer of nucleotides is intended.

A polynucleotide which encodes a gene product, e.g., a polypeptide, can include a promoter and/or other transcription or translation control elements operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other transcription control elements, besides a promoter, for example enhancers, operators, repressors, and transcription termination signals, can also be operably associated with a coding region to direct gene product expression.

“Transcriptional control sequences” refer to DNA regulatory sequences, such as promoters, enhancers, terminators, and the like, that provide for the expression of a coding sequence in a host cell. A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit beta-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence),

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.”

Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell,

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. The Smith-Waterman is one type of algorithm that permits gaps in sequence alignments. See Meth. Mot. Biol. 70: 173-187 (1997). Also, the GAP program using the Needleman and Wunsch alignment method can be utilized to align sequences. See J. Mol. Biot. 48: 443-453 (1970).

The term “amino acid substitution” and its synonyms described above are intended to encompass modification of an amino acid sequence by replacement of an amino acid with another, substituting, amino acid. The substitution may be a conservative substitution. Tt may also be a non-conservative substitution. The term conservative, in referring to two amino acids, is intended to mean that the amino acids share a common property recognized by one of skill in the art. For example, amino acids having hydrophobic nonacidic side chains, amino acids having hydrophobic acidic side chains, amino acids having hydrophilic nonacidic side chains, amino acids having hydrophilic acidic side chains, and amino acids having hydrophilic basic side chains. Common properties may also be amino acids having hydrophobic side chains, amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Methods for replacing an amino acid are well known to the skilled in the art and include, but are not limited to, mutations of the nucleotide sequence encoding the amino acid sequence. Reference to “one or more” herein is intended to encompass the individual embodiments of, for example, 1, 2, 3, 4, 5, 6, or more.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease (and/or symptoms caused by the disease) from occurring in a subject which may be predisposed to the disease or at risk of acquiring the disease but has not yet been diagnosed as having it; (b) inhibiting the disease (and/or symptoms caused by the disease), i.e., arresting its development; and (c) relieving the disease (and/or symptoms caused by the disease), i.e., causing regression of the disease (and/or symptoms caused by the disease), i.e., ameliorating the disease and/or one or more symptoms of the disease.

As used herein, the term “treating complement factor H disorders” may encompass alleviating, reducing, and/or ameliorating symptoms, and/or preventing the development of additional symptoms associated with complement factor H disorder, which can manifest as several different phenotypes, including asymptomatic, recurrent bacterial infections, and renal failure. This is typically characterized by decreased serum levels of factor H, complement component C3, and a decrease in other terminal complement components, indicating activation of the alternative complement pathway. This disorder is associated with a number of renal diseases with variable clinical presentation and progression, including C3 glomerulopathy and atypical hemolytic uremic syndrome. Also provided herein are compositions and methods for treating one or more of dry age related macular degeneration (AMD), geographic atrophy secondary to AMD, atypical hemolytic uremic (including, e.g., syndrome microangiopathic haemolytic anemia, thrombocytopenia. acute renal failure), paroxysmal nocturnal hemoglobinuria (PNH), schizophrenia, ischemic stroke, and/or preventing or treating bacterial infections caused by recruitment of bacterial pathogens (e.g., Aspergillus spp.; Borrelia burgdorferi; H. duttonii; B. recurrentis; Candida albicans; Francisella tularensis; Haemophilus influenzae; Neisseria meningitidis; Streptococcus pyogenes, or one of the five factor H binding proteins of B. burgdorferi(CRASP-1, CRASP-2, CRASP-3, CRASP-4, or CRASP-5), among others.

As used herein, the term “treating complement associated disorders” includes alleviating, reducing, and/or ameliorating symptoms, both of the complement factor H disorders identified above, but also other disorders associated with uncontrolled alternative pathway complement regulation.

“Complement-mediated disorders” may encompass symptoms associated with complement dysregulation which can manifest as several different phenotypes, including asymptomatic, recurrent bacterial infections, and various tissue injuries including but not limited to renal diseases. Unless otherwise specified, both homozygous subjects and heterozygous subjects are encompassed within this definition. Complement dysregulation is typically caused by loss of function mutations in, or auto-antibodies against, complement regulatory proteins including but not limited to fH, factor I (fI) and membrane cofactor protein (MCP) or by gain of function mutations in other complement proteins including but not limited to C3 and factor B (fB). Complement dysregulation is typically, though not always, characterized by decreased serum levels of factor H, complement component C3, fB and a decrease in other terminal complement components, indicating activation of the alternative and/or the terminal complement pathway. Complement-mediated pathologies that can be treated by the present invention of composition and method include but are not limited to the following diseases with variable clinical presentation and progression: C3 glomerulopatby (formally called membranoproliferative glomerulonephritis type11 or MPGNII), of which there are two known forms—dense deposit disease (DDD) and C3 glomerulonephritis (C3GN); thrombotic microangiopathy (TMA) including but not limited to atypical hemolytic uremic syndrome (aHUS), Shiga-like toxin-producing E. coli HUS (STEC-HUS) and thrombotic throtribocytopenia purpura (TTP); retinal degenerative eye disease including age related macular degeneration (AMD), RPE degeneration, chorioretinal degeneration, photoreceptor degeneration, paroxysmal nocturnal hemoglobinuria (PNH), ischemia reperfusion injury of all organs and settings, rheumatoid arthritis, hemodialysis, diabetic nephropathy, diabetic vasculopathy, asthma, systemic lupus erythematosus (SLE), ischemic stroke, abdominal aortic aneurysm (AAA), anti-neutrophil cytoplasmic antibody (ANCA) mediated vasculitis (ANCA vasculitis), ANCA-mediated hemorrhagic lung injury and disease, ANCA glomerulonephritis, graft versus host disease (GvHD), acute or delay graft rejection in organ transplantation, Crohn's disease, psoriasis, multiple sclerosis, anti-phospholipid syndrome, preeclampsia, atherosclerosis, neuromyelitis optica (NMO), autoimmune skin-blistering disease, Bullous pemphigoid (BP), Alzheimer's disease (AD), as well as bacterial infections caused by recruitment of bacterial pathogens (e.g., Aspergillus spp.; Borrelia burgdorferi; B. duttonii; . recurrentis; Candida albicans; Fr and sella tularensis; Haeimophilus influenzae; Neisseria meningitidis; Streptococcus pygenes).

The terms “individual,” “host,” “subject,” and “patient” are used interchangeably herein, and refer to a mammal, including, but not limited to, humans; non-human primates, including simians; mammalian sport animals (e.g., horses); mammalian farm animals (e.g., sheep, goats, etc.); mammalian pets (dogs, cats, etc.); and rodents (e.g., mice, rats, etc.).

The term “effective amount” as used herein is an amount sufficient to effect beneficial or desired clinical results. An effective amount can be administered in one or more administrations. For purposes of this disclosure, an effective amount of a compound (e.g., an infectious rAAV virion) is an amount that is sufficient to palliate, ameliorate, stabilize, reverse, prevent, slow or delay the progression of (and/or symptoms associated with) a particular disease state (e.g., a disorder associated with complement dysfunction). Accordingly, an effective amount of an infectious rAAV virion is an amount of the infectious rAAV virion that is able to effectively deliver a heterologous nucleic acid to a target cell (or target cells) of the individual, Effective amounts may be determined preclinically by, e.g., detecting in the cell or tissue the gene product (RNA, protein) that is encoded by the heterologous nucleic acid sequence using techniques that are well understood in the art, e.g. RT-PCR, western blotting, ELISA, fluorescence or other reporter readouts, and the like. Effective amounts may be determined clinically by, e.g. detecting a change in the onset or progression of disease using methods known in the art, e.g. 6-minute walk test, left ventricular ejection fraction, hand-held dynamometry, Vignos Scale and the like as described herein and as known in the art.

### DETAILED DESCRIPTION

Novel rAAV virions encoding engineered factor H (fH) genes and protein variants are described herein. These rAAV virions are characterized by durable and robust expression of fH protein in the retina and increased efficacy in treating conditions associated with factor H and other complement disorders.

Delivery of these rAAV virions to subjects in need thereof may be achieved via a number of routes, preferably by intravitreal administration. Also provided are methods of using these rAAV virions in regimens for treating factor H associated disorders, particularly dry AMD and geographic atrophy secondary to AMD.

### Heterologous Nucleic Acid

The novel rAAV virions described herein comprise a heterologous nucleic acid encoding a human factor H (hfH) variant, operably linked to an expression control sequence. Typically, the heterologous nucleic acid includes an AAV genome with the rep and cap genes deleted and/or replaced by the hfH sequence and its associated expression control sequences. The hfH sequence is typically inserted adjacent to one or two (i.e., is flanked by) AAV TRs or TR elements adequate for viral replication (Xiao et al., 1997, J. Virol. 71(2): 941-948), in place of the nucleic acid encoding viral rep and cap proteins. Other regulatory sequences suitable for use in facilitating tissue-specific expression of the hFH gene sequence in the target cell (e.g., a retinal cell) may also be included.

In some aspects, the rAAV virion comprises a heterologous nucleic acid comprising (a) an AAV2 terminal repeat (b) a transcription control sequence (c) a nucleotide sequence encoding an hfl variant as herein described (d) a polyadenylation sequence and (e) an AAV2 terminal repeat.

### hfH Genes

The amino acid sequence of the mature “wild-type” human complement factor H (isoform 1) is provided at www.uniprot.org/uniprot/P08603 and serves as a reference for the amino acid numbering of the hfH isoform 1 [see also SEQ ID NO: 39 of U.S. Pat. No. 10,988,519, the entire contents of which are incorporated herein by reference]:

(SEQ ID NO: 3)
MRLLAKIICLMLWAICVAEDCNELPPRRNTEILTGSWSDQTYPEGTQAIYKCRPGYRS
LGNVIMVCRKGEWVALNPLRKCQKRPCGHIPGDTPFGTFTLTGGNVFEYGVKAVYT
CNEGYQLLGEINYRECDTDGWTNDIPICEVVKCLPVTAPENGKIVSSAMEPDREYHF
GQAVRFVCNSGYKIEGDEEMHCSDDGFWSKEKPKCVEISCKSPDVINGSPISQKIIYK
ENERFQYKCNMGYBYSERGDAVCTESGWRPLPSCEEKSCDNPYIPNGDYSPLRIKHR
TGDEITYQCRNGFYPATRGNTAKCTSTGWIPAPRCTLKPCDYPDIKHGGLYHENMRR
PYFPVAVGKYYSYYCDEHFETPSGSYWDHIHCTQDGWSPAVPCLRKCYFPYLENGY
NQNHGRKFVQGKSIDVACHPGYALPKAQTTVTCMENGWSPTPRCIRVKTCSKSSIDI
ENGFISESQYTYALKEKAKYQCKLGYVTADGETSGSITCGKDGWSAQPTCIKSCDIP
VFMNARTKNDFTWFKLNDTLDYECHDGYESNTGSTTGSIVCGYNGWSDLPICYERE
CELPKIDVHLVPDRKKDQYKVGEVLKFSCKPGFTIVGPNSVQCYHFGLSPDLPICKEQ
VQSCGPPPELLNGNVKEKTKEEYGHSEVVEYYCNPRFLMKGPNKIQCVDGEWTTLP
VCIVEESTCGDIPELEHGWAQLSSPPYYYGDSVEFNCSESFTMIGHRSITCIHGVWTQL
PQCVAIDKLKKCKSSNLIILEEHLKNKKEFDHNSNIRYRCRGKEGWIHTVCINGRWDP
EVNCSMAQIQLCPPPPQIPNSHNMTTTLNYRDGEKVSVLCQENYLIQEGEEITCKDGR
WQSIPLCVEKIPCSQPPQIEHGTINSSRSSQESYAHGTKLSYTCEGGFRISEENETTCYM
GKWSSPPQCEGLPCKSPPEISHGVVAHMSDSYQYGEEVTYKCFEGFGIDGPAIAKCL
GEKWSHPPSCIKTDCLSLPSFENAIPMGEKKDVYKAGEQVTYTCATYYKMDGASNV
TCINSRWTGRPTCRDTSCVNPPTVQNAYIVSRQMSKYPSGERVRYQCRSPYEMFGDE
EVMCLNGNWTEPPQCKDSTGKCGPPPPIDNGDITSFPLSVYAPASSVEYQCONLYQL
EGNKRITCRNGQWSEPPKCLHPCVISREIMENYNIALRWTAKQKLYSRTGESVEFVC
KRGYRLSSRSHTLRTTCWDGKLEYPTCAKR

The leader sequence (MRLLAKIICLMLWAICVA; SEQ ID NO:4) is located at amino acids 1 to 18 of factor H, with reference to SEQ ID NQ:3. The mature (secreted) M protein is located at amino acids 19 to 1231 of SEQ ID NO:3. There are alternative methods of determining the location of the 20 short complement repeats (SCRs). The location of the domains referred to herein is based on the numbering used in C. Estaller et al, Eur J Immunol, 1991 Mar; 21 (3):799-802.

The amino acid sequence of each of the 20 SCRs of hfH is provided at Table 1 below (see also SEQ ID Nos: 3, 5, 7, 9, 11, 13, 14, 16, 17, 19, 21, 23, 25, 27, 29, 31, 33, 35, 37 and 38 of U.S. Pat. No. 10,988,519):

| SCR | Amino acid Sequence | SCR | Amino acid sequence |
|---|---|---|---|
| SCR1 | EDCNELPPRRNTEILTGSW | SCR11 | CGPPPELLNGNVKEKTKEEYG |
| | SDQTYPEGTQAIYKCRPGY | | HSEVVEYYCNPRFLMKGPNKI |
| | RSLGNIIMVCRKGEWVAL | | QCVDGEWTTLPVC (SEQ ID |
| | NPLRKC (SEQ ID NO: 5) | | NO: 15) |
| SCR2 | CGHPGDTPFGTFTLTGGNV | SCR12 | CGDIPELEHGWAQLSSPPYYY |
| | FEYGVKAVYTCNEGYQLL | | GDSVEFNCSESFTMIGHRSITCI |
| | GEINYRECDTDGWTNDIPI | | HGVWTQLPQC (SEQ ID |
| | C (SEQ ID NO: 6) | | NO: 16) |
| SCR3 | CLPVTAPENGKIVSSAMEP | SCR13 | CKSSNLIILEEHLKNKKEFDHN |
| | DREYHFGQAVRFVCNSGY | | SNIRYRCRGKEGWIHTVCING |
| | KIEGDEEMHCSDDGFWSK | | RWDPEVNC (SEQ ID NO: 17) |
| | EKPKC (SEQ ID NO: 7) | | |
| SCR4 | CKSPDVINGSPISQKIIYKE | SCR14 | CPPPPQIPNSHNMTTTLNYRD |
| | NERFQYKCNMGYEYSERG | | GEKVSVLCQENYLIQEGEEITC |
| | DAVCTESGWRPLPSC (SEQ | | KDGRWQSIPLC (SEQ ID |
| | ID NO: 8) | | NO: 18) |
| SCR5 | CDNPYIPNGDYSPLRIKHR | SCR15 | CSQPPQIEHGTINSSRSSQESYA |
| | TGDEITYQCRNGFYPATRG | | HGTKLSYTCEGGFRISEENETT |
| | NTAKCTSTGWIPAPRC | | CYMGKWSSPPQC (SEQ ID |
| | (SEQ ID NO: 9) | | NO: 19) |
| SCR6 | CDYPDIKHGGLYHENMRR | SCR16 | CKSPPEISHGVVAHMSDSYQY |
| | PYFPVAVGKYYSYYCDEH | | GEEVTYKCFEGFGIDGPAIAKC |
| | FETPSGSYWDHIHCTQDG | | LGEKWSHPPSC (SEQ ID |
| | WSPAVPC (SEQ ID NO: 10) | | NO: 20) |
| SCR7 | CYFPYLENGYNQNYGRKF | SCR17 | CLSLPSFENAIPMGEKKDVYK |
| | VQGKSIDVACHPGYALPK | | AGEQVTYTCATYYKMDGASN |
| | AQTTVTCMENGWSPTPRC | | VTCINSRWTGRPTC (SEQ ID |
| | (SEQ ID NO: 11) | | NO: 21) |
| SCR8 | CSKSSIDIENGFISESQYTY | SCR18 | CVNPPTVQNAYIVSRQMSKYP |
| | ALKEKAKYQCKLGYVTAD | | SGERVRYQCRSPYEMFGDEEV |
| | GETSGSITCGKDGWSAQPT | | MCLNGNWTEPPQC (SEQ ID |
| | C (SEQ ID NO: 12) | | NO: 22) |
| SCR9 | CDIPVFMNARTKNDFTWF | SCR19 | CGPPPPIDNGDITSFPLSVYAPA |
| | KLNDTLDYECHDGYESNT | | SSVEYQCONLYQLEGNKRITC |
| | GSTTGSIVCGYNGWSDLPI | | RNGQWSEPPKC (SEQ ID |
| | C (SEQ ID NO: 13) | | NO: 23) |
| SCR10 | CELPKIDVHLVPDRKKDQY | SCR20 | CVISREIMENYNIALRWTAKQ |
| | KVGEVLKFSCKPGFTIVGP | | KLYSRTGESVEFVCKRGYRLS |
| | NSVQCYHFGLSPDLPIC | | SRSHTLRTTCWDOKLEYPTCA |
| | (SEQ ID NO: 14) | | KR (SEQ ID NO: 24) |

Optionally, an engineered hfH variant as described herein may have a heterologous leader sequence substituted for the native hfH leader sequence. Additionally, or optionally, another hfH isoform (e.g., isoform 2), the sequence of which are available, e.g., from http://www.uniprot.org/uniprot/P08603, and/or one of the natural amino acid variants therein which are not associated with a disorder, See, SEQ ID NO: 40 of U.S. Pat. No. 10,988,519. In the following descriptions, substitutions may be written as (first amino acid identified by single letter code)-residue position #—(second amino acid identified by single letter code) whereby the first amino acid is the substituted amino acid and the second amino acid is the substituting amino acid at the specified position with reference to isoform 1; however, by conventional alignment steps, the corresponding amino acid residues identified herein with respect to the numbering of isoform 1 can be located in isoform 2 and non-disease causing natural variants of the SCRs of isoform 1 or 2 of fH.

An rAAV as described herein comprises a heterologous nucleic acid encoding a functional fH variant. Examples of functional fH variants include those having SCR1-4 and 19-20 of the fH protein, with one or more of an SCR7, SCR17 or SCR18 domain. Further variants include those having one or more of SCR6, SCR8, SCR16, SCR17, SCR18, or fragments thereof, and combinations thereof. For example, such variants may include, e.g., fH SCR1-4, 6-8, 19-20; fH SCR1-4, 6-8, 18-20; fH SCR1-4, 6-8, 17-20; fH SCR1-4, 6-7, 19-20; fH SCR1-4, 6-7, 18-20; fH SCR1-4, 6-7, 17-20; fH SCR1-4, 7-8, 19-20; fH SCR1-4, 7-8, 18-20; fH SCR1-4, 7-8, 17-20; fH SCR1-4, 7, 19-20; fH SCR1-4, 7, 18-20; fH SCR1-4, 7, 17-20; SCR1-4, 17, 19-20; SCR1-4, 18-20; SCR1-4, 17-20 and/or fH SCR1-4, 7, 16-20, among others. In certain embodiments, the hfH variant further comprises additional hfH SCRs, e.g., SCR 6, SCRS, SCR16, or combinations thereof. In preferred embodiments, hfH SCR5 is absent. However, in certain embodiments, hfH SCR5 may be present in whole or a fraction thereof. In certain embodiments, hfH SCR9, SCR10, SCR11, SCR12, SCR13, SCR14, and/or SCR 15 are absent, or are at least functionally deleted. Optionally, one or more of the SCRS in these variants may be a “functional fragment” of the SCRs, rather than a full-length SCR. By “functional fragment” it is meant an amino acid sequence (or coding sequence therefor) less than the full-length SCR which is characterized by having one or more of complement inhibiting activity, the ability to bind, heparin, and/or C3b-binding activity.

With the hfH variants, domains may be located immediately adjacent to one another (e.g., the carboxy terminus of one domain may immediately follow the amino terminus of the preceding domain). Alternatively, one or more of the SCR domains may have a linker composed of one to about 12 to 18 amino acids located between them. For example, a variant may contain SCR1-(L)-SCR2-(L)-SCR3-(L)-SCR4-(L)-(SCR6-(L))-SCR7-(L)-(SCR8-(L))-(SCR16-(L))-(SCR17-(L))-(SCR18-(LS))-SCR19-(L)-SCR20, wherein the ( ) indicate optional component, “L” refers to a linker, which may be absent or independently selected from an amino acid sequence of about 1 to about 12-18 amino acids. In other words, where a variant contains multiple linkers, each of the linkers may have the same sequence or a different sequence. In certain embodiments, a variant contains at least one, at least two, at least three, at least four, at least five linkers, at least six linkers. Examples of suitable linkers include the natural linkers described herein or artificial linkers. Each of these wild-type linkers may be located in their native position. Alternatively, one or more of these wild-type linkers may be used in a different linker position, or in multiple different linker positions.

Optionally, one or more of these linkers may be fH sequences and are independently selected. Alternatively, one or more of the linkers may be heterologous to fH, e.g., from a different source, whether artificial, synthetic, or from a different protein which confers suitable flexibility to the fH variant. Examples of other suitable linkers may include, e.g., a poly Gly linker and other linkers providing suitable flexibility (e.g., http://parts.igem.org/rotein_domainsitinker), which is incorporated by reference herein. In certain embodiments, the linkers lack any fH function.

Some representative suitable fH linker sequences are provided in Table 2 below

| Linker Sequence | Exemplary linkage |
|---|---|
| QKRP (SEQ ID NO: 25) | SCR1-SCR2 |
| EVVK (SEQ ID NO: 26) | SCR2-SCR3 |
| VEIS (SEQ ID NO: 27) | SCR3-SCR4 |
| EEKSTLKP (SEO ID NO: 28) | SCR4-SCR6 |
| LRK | SCR6-SCR7 |
| IRVKT (SEQ ID NO: 29) | SCR7-SCR8 |
| IKTD (SEQ ID NO: 30) | SCR8-SCR17 |
| IKSKDSTGK (SEQ ID NO: 31) | SCR8-SCR19 |
| RDTS (SEQ ID NO: 32) | SCR17-SCR18 |
| KDSTGK (SEQ ID NO: 33) | SCR18-SCR19 |
| LHP | SCR19-SCR20 |

In some preferred embodiments, the rAAV comprises a heterologous nucleic acid encoding an hfH variant with the following structure; SCR1-(L1)-SCR2-(L2)-SCR3-(L3)-SCR4-(L4)-SCR6-(L5)-SCR7-(L6)-SCR8-(L7)-SCR17-(LS)-SCR18-(L9)-SCR19-(L10)-SCR20.

In a particularly preferred embodiment, the heterologous nucleic acid encodes an fH variant having the following amino acid sequence:

(SEQ ID NO: 34)
MRLLAKIICLMLWAICVAEDCNELPPRRNTEILTGSWSDQTYPEGTQAIYKCRPGYRS
LGNIIMVCRKGEWVALNPLRKCQKRPCGHPGDTPFGTFTLTGGNVFBYGVKAVYTC
NEGYQLLGEINYRECDTDGWTNDIPICEVVKCLPVTAPENGKIVSSAMEPDREYHFG
QAVRFVCNSGYKIEGDEEMHCSDDGFWSKEKPKCVEISCKSPDVINGSPISQKIIYKE
NERFQYKCNMGYEYSERGDAVCTESGWRPLPSCEEKSTLKPCDYPDIKHGGLYHEN
MRRPYFPVAVGKYYSYYCDEHFETPSGSYWDHIHCTQDGWSPAVPCLRKCYFPYLE
NGYNQNYGRKFVQGKSIDVACHPGYALPKAQTTVTCMENGWSPTPRCIRVKTCSKS
SIDIENGFISESQYTYALKEKAKYQCKLGYVTADGETSGSITCGKDGWSAQPTCIKSI
KTDCLSLPSFENAIPMGEKKDVYKAGEQVTYTCATYYKMDGASNVTCINSRWTGRP
TCRDTSCVNPPTVQNAYIVSRQMSKYPSGERVRYQCRSPYEMFGDEEVMCLNGNW
TEPPQCKDSTGKCGPPPPIDNGDITSFPLSVYAPASSVEYQCONLYQLEGNKRITCRN
GQWSEPPKCLHPCVISREIMENYNIALRWTAKQKLYSRTGESVEFVCKRGYRLSSRS
HTLRTTCWDGKLEYPTCAKR

In other embodiments, the rAAV comprises a heterologous nucleic acid encoding an hfH variant having the following SCR1-(L1)-SCR2-(L2)-SCR3-(L3)-SCR4-(A4)-SCR6-(L5)-SCR7-(L6)-SCR8-(L7)-SCR19-(L10)-SCR20.

These and other variants may include other fH sequences. For example, the coding sequence of the fH variant may also include a leader sequence. Such a leader sequence may be an fH leader (e.g., MRLLAKIICLMLWAICVA; SEQ ID NO:4). Optionally, the leader sequence can be from another source, e.g., an IL-2 leader, [see, e.g., the index of mammalian leader sequences identified in www.signalpeptide.de/J, incorporated by reference herein. In one embodiment, the leader sequence selected is less than about 26 amino acids in length (e.g., from about 1 to about 26 amino acids), more preferably less than 20 amino acids (from about 1 to about 20 amino acids), and most preferably, less than about 18 amino acids in length (from about 1 to about 18 amino acids). By “functional deletion” is meant an amino acid sequence (or coding sequence therefor) which lacks complement inhibiting activity, the C3b-binding activity, and optionally also further lacks heparin binding activity.

In addition to the fH protein variants provided herein, nucleic acid sequences encoding these fH protein variants are provided. The coding sequences for these variants may be from wild-type sequences of the leader sequence and/or one or more SCRs of isoform 1, isoform 2, or non-disease associated variants. Alternatively or additionally, web-based or commercially available computer programs, as well as service based companies may be used to back translate the amino acids sequences of the leader sequence, and/or one or more of the SCRs to nucleic acid coding sequences, including both RNA and/or cDNA. See, e.g., backtranseq by EMBOSS, Gene Infinity (www.geneinfinity.org/sms-/sms_backtranslation.html); ExPasy (www.expasy.org/tools/).

In one embodiment, the RNA and/or cDNA coding sequences are designed for optimal expression in human cells.

Codon-optimized coding regions can be designed by various different methods. This optimization may be performed using methods which are available on-line, published methods, or a company which provides codon optimizing services. One codon optimizing method is described, e.g., in WO 2015/012924 A2, which is incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

In another embodiment, at least one glycosylation site is engineered into at least one of the SCRs present in the hfH variant, at least two of the SCRs present in the hfH variant, at least three of the SCRs present in the hfH variant, or more. For example, the glycosylation site may be engineered into one or more of SCR1, SCR2, SCR3, SCR4, SCR19, and/or SCR20. In another embodiment, SCR17 and/or SCR18 are additionally or alternatively glycosylated. in still a further embodiment, SCR4, 17 and 18 are glycosylated. In a preferred embodiment, the hfH variant comprises or consists of or consists essentially of SCR1-4, SCR6-8 and SCF17-19 and SCR17 and SCR18 are glycosylated. In certain embodiments, a glycosylation site may be engineered into a linker. However, in such instance, the linker is preferably at least six amino acids in length up to about 18 amino acids in length, e.g., 8-18, 10-15, or 12 amino acids.

As used herein, a glycosylation site refers to the point of attachment of oligosaccharides to a carbon atom (C-linked), nitrogen atom. (N-linked), or oxygen atom (0-linked), or glycation (non-enzymatic attachment of reducing sugars to the nitrogen atom of a protein (e.g., the nitrogen atom of an asparagine (Asn) side chain that is part of an Asn-X-Ser/Thr, wherein X is any amino acid except Pro). In certain embodiments, N-glycosylation sites are desired. A variety of techniques are known in the art for engineering N-glycosylation sites. See. e.g. Y Liu et al, Biotech Prog 2009 September-October; 25(5): 1468-1475; Sala R J, Griebenos K. Glycoslylation of therapeutic proteins: an effective strategy to optimize efficacy. BioDrugs. 2010 Feb. 1; 24(1): 9-21.

Exemplary hfH variant nucleotide sequences encoding the amino acid sequence of SEQ ID NO:34 are provided below:

(SEQ ID NO: 35)
ATGCGCCTGCTGGCCAAGATCATCTGCCTGATGCTGTGGGCCATCTGCGTGGCCG
AGGACTGTAACGAGCTGCCCCCTCGGAGAAATACAGAGATCCTGACCGGCTCTT
GGAGCGATCAGACCTACCCAGAGGGCACACAGGCCATCTACAAGTGCAGGCCCG
GCTATCGCTCCCTGGGCAATATCATCATGGTGTGCAGGAAGGGAGAGTGGGTGG
CCCTGAACCCACTGAGAAAGTGCCAGAAGAGGCCATGTGGACACCCAGGCGACA
CACCTTTCGGCACCTTTACACTGACCGGCGGCAACGTGTTCGAGTACGGCGTGAA
GGCCGTGTATACCTGCAATGAGGGCTACCAGCTGCTGGGCGAGATCAACTACCG
GGAGTGTGACACAGATGGCTGGACCAATGATATCCCCATCTGCGAGGTGGTGAA
GTGTCTGCCAGTGACCGCCCCCGAGAACGGCAAGATCGTGAGCTCCGCCATGGA
GCCTGACAGAGAGTATCACTTCGGCCAGGCCGTGAGGTTCGTGTGCAATAGCGG
CTACAAGATCGAGGGCGATGAGGAGATGCACTGTTCCGACGATGGCTTCTGGTC
TAAGGAGAAGCCCAAGTGCGTGGAGATCTCCTGTAAGTCTCCTGACGTGATTAA
CGGCAGCCCAATCTCCCAGAAGATCATCTATAAGGAGAATGAGCGGTTTCAGTA
CAAGTGCAACATGGGCTACGAGTATTCCGAGAGAGGCGATGCCGTGTGCACAGA
GTCTGGATGGAGGCCCCTGCCTAGCTGCGAGGAGAAGTCCACCCTGAAGCCTTG
TGACTATCCAGATATCAAGCACGGCGGCCTGTATCACGAGAATATGAGGCGCCC
TTACTTCCCAGTGGCCGTGGGCAAGTACTATTCCTACTATTGCGACGAGCACTTT
GAGACACCATCCGGCTCTTACTGGGACCACATCCACTGTACCCAGGATGGCTGGT
CTCCCGCCGTGCCTTGCCTGAGAAAGTGTTACTTCCCCTATCTGGAGAACGGCTA
CAACCAGAATTATGGCAGGAAGTTTGTGCAGGGCAAGTCCATCGACGTGGCATG
CCACCCAGGATACGCACTGCCTAAGGCACAGACCACAGTGACCTGTATGGAGAA
TGGCTGGAGCCCAACACCCAGGTGCATCCGCGTGAAGACCTGTTCCAAGAGCAG
CATCGACATCGAGAACGGCTTCATCAGCGAGTCCCAGTACACCTATGCCCTGAA
GGAGAAGGCCAAGTATCAGTGCAAGCTGGGATACGTGACCGCAGACGGAGAAA
CATCTGGCAGCATCACCTGCGGCAAGGATGGCTGGTCTGCCCAGCCCACATGCAT
CAAGAGCATCAAGACCGACTGTCTGTCTCTGCCCAGCTTTGAGAATGCCATCCCT
ATGGGCGAGAAGAAGGACGTGTACAAGGCCGGCGAGCAGGTGACATACACCTG
CGCCACATACTATAAGATGGACGGCGCCAGCAACGTGACCTGTATCAATTCCCG
GTGGACAGGCAGACCTACCTGCAGGGATACAAGCTGCGTGAACCCACCCACCGT
GCAGAATGCCTATATCGTGTCCCGCCAGATGTCTAAGTACCCTAGCGGCGAGCG
GGTGAGATATCAGTGCCGGTCCCCATACGAGATGTTCGGCGACGAGGAAGTGAT
GTGCCTGAACGGCAATTGGACAGAGCCTCCACAGTGCAAGGATAGCACCGGCAA
GTGTGGACCACCTCCACCAATCGACAACGGCGATATCACATCCTTTCCACTGAGC
GTGTACGCCCCCGCCTCCTCTGTGGAGTATCAGTGCCAGAACCTGTACCAGCTGG
AGGGCAATAAGCGCATCACCTGTCGGAACGGCCAGTGGTCTGAGCCTCCAAAGT
GCCTGCACCCTTGCGTGATCTCCAGAGAGATCATGGAGAACTATAATATCGCCCT
GCGCTGGACAGCCAAGCAGAAGCTGTACTCTCGGACCGGCGAGAGCGTGGAGTT
CGTGTGCAAGCGGGGCTATAGACTGAGCTCCCGCAGCCACACACTGCGGACCAC
ATGCTGGGACGGCAAGCTGGAGTACCCAACCTGTGCCAAGAGGTGA
(SEQ ID NO: 36)
ATGAGGCTGCTGGCCAAGATCATCTGCCTGATGCTGTGGGCCATCTGTGTGGCTG
AGGACTGTAATGAGCTGCCCCCTAGGAGAAATACAGAGATCCTGACTGGCTCTT
GGTCTGATCAGACCTACCCAGAGGGCACACAGGCCATCTACAAGTGCAGGCCTG
GCTATAGATCCCTGGGCAATATCATCATGGTGTGCAGGAAGGGAGAGTGGGTGG
CCCTGAACCCACTGAGAAAGTGCCAGAAGAGGCCATGTGGACACCCAGGAGACA
CACCTTTTGGCACCTTTACACTGACAGGGGGCAATGTGTTTGAGTATGGTGTGAA
GGCAGTGTATACCTGCAATGAGGGCTACCAGCTGCTGGGAGAGATCAACTACAG
GGAGTGTGACACAGATGGCTGGACCAATGATATCCCCATCTGTGAGGTGOTGAA
GTGTCTGCCAGTGACTGCCCCTGAGAATGGCAAGATAGTGAGCTCTGCCATGGA
GCCTGACAGAGAGTATCACTTTGGCCAGGCTGTGAGGTTTGTGTGCAATAGTGGC
TACAAGATTGAGGGGGATGAGGAGATGCACTGTTCAGATGATGGCTTCTGGTCT
AAGGAGAAGCCCAAGTGTGTGGAGATCTCCTGTAAGTCTCCTGATGTGATTAATG
GCAGCCCAATCTCCCAGAAGATCATCTATAAGGAGAATGAGAGATTTCAGTACA
AGTGCAACATGGGCTATGAGTATTCAGAGAGAGGTGATGCTGTGTGCACAGAGT
CTGGATGGAGGCCCCTGCCTAGCTGTGAGGAGAAGTCCACCCTGAAGCCTTGTG
ACTATCCAGATATCAAGCATGGAGGCCTGTATCATGAGAATATGAGGAGGCCTT
ACTTCCCAGTGGCTGTGGGCAAGTACTATTCCTACTATTGTGATGAGCACTTTGA
GACACCATCTGGCTCTTACTGGGACCACATCCACTGTACCCAGGATGGCTGGTCT
CCTGCTGTGCCTTGCCTGAGAAAGTGTTACTTCCCCTATCTGGAGAATGGCTACA
ACCAGAATTATGGCAGGAAGTTTGTGCAGGGCAAGTCCATTGATGTGGCATGCC
ACCCAGGATATGCACTGCCTAAGGCACAGACCACAGTGACCTGTATGGAGAATG
GCTGGAGCCCAACACCCAGGTGCATCAGGGTGAAGACCTGTTCCAAGAGCAGCA
TTGACATAGAGAATGGCTTCATCTCAGAGTCCCAGTACACCTATGCCCTGAAGGA
GAAGGCCAAGTATCAGTGCAAGCTGGGATATGTGACAGCAGATGGAGAAACATC
TGGCAGCATCACCTGTGGCAAGGATGGCTGGTCTGCCCAGCCCACATGCATCAA
GAGCATCAAGACTGACTGTCTGTCTCTGCCCAGCTTTGAGAATGCCATCCCTATG
GGTGAGAAGAAGGATGTGTACAAGGCTGGAGAGCAGGTGACATACACCTGTGCC
ACATACTATAAGATGGATGGAGCCAGCAATGTGACCTGTATCAATTCCAGATGG
ACAGGCAGACCTACCTGCAGGGATACAAGCTGTGTGAACCCACCCACTGTGCAG
AATGCCTATATTGTGTCCAGGCAGATGTCTAAGTACCCTTCTGGAGAGAGAGTGA
GATATCAGTGCAGATCCCCATATGAGATGTTTGGGGATGAGGAAGTGATGTGCC
TGAATGGCAATTGGACAGAGCCTCCACAGTGCAAGGATAGCACTGGCAAGTGTG
GACCACCTCCACCAATTGACAATGGAGATATCACATCCTTTCCACTGTCAGTGTA
TGCCCCTGCCTCCTCTGTGGAGTATCAGTGCCAGAACCTGTACCAGCTGGAGGGC
AATAAGAGAATCACCTGTAGAAATGGCCAGTGGTCTGAGCCTCCAAAGTGCCTG
CACCCTTGTGTGATCTCCAGAGAGATCATGGAGAACTATAATATAGCCCTGAGGT
GGACAGCCAAGCAGAAGCTGTACTCTAGGACTGGGGAGTCAGTGGAGTTTGTGT
GCAAGAGAGGCTATAGACTGAGCTCCAGAAGCCACACACTGAGGACCACATGCT
OGGATGGCAAGCTGGAGTACCCAACCTGTGCCAAGAGGTGA
(SEQ ID NO: 37)
ATGAGACTTCTAGCAAAGATTATTTGCCTTATGTTATGGGCTATTTGTGTAGCAG
AAGATTGCAATGAACTTCCTCCAAGAAGAAATACAGAAATTCTGACAGGTTCCT
GGTCTGACCAAACATATCCAGAAGGCACCCAGGCTATCTATAAATGCAGGCCTG
GATATAGATCTCTTGGAAATATCATAATGGTATGCAGGAAGGGAGAATGGGTTG
CTCTTAATCCATTAAGGAAATGTCAGAAAAGGCCCTGTGGACATCCTGGAGATA
CTCCTTTTGGTACTTTTACCCTTACAGGAGGAAATGTGTTTGAATATGGTGTAAA
AGCTGTGTATACATGTAATGAGGGGTATCAATTGCTAGGTGAGATTAATTACAGG
GAATGTGACACAGATGGATGGACCAATGATATTCCTATATGTGAAGTIGTGAAG
TGTTTACCAGTGACAGCACCAGAGAATGGAAAAATTGTCAGTAGTGCAATGGAA
CCAGATAGAGAATACCATTTTGGACAAGCAGTAAGATTTGTATGTAACTCAGGCT
ACAAGATTGAAGGAGATGAAGAAATGCATTGTTCAGATGATGGTTTTTGGAGTA
AAGAGAAACCAAAGTGTGTGGAAATTTCATGCAAATCCCCAGATGTTATAAATG
GATCTCCTATATCTCAGAAGATTATTTATAAGGAGAATGAAAGATTTCAATATAA
ATGTAACATGGGTTATGAATACAGTGAAAGAGGAGATGCTGTATGCACTGAATC
TGGATGGAGACCTTTGCCTTCATGTGAAGAAAAATCAACCTTGAAACCTTGTGAT
TATCCAGACATTAAACATGGAGGTCTATATCATGAGAATATGAGAAGACCATAC
TTTCCAGTAGCTGTAGGAAAATATTACTCCTATTACTGTGATGAACATTTTGAGA
CTCCTTCAGGAAGTTACTGGGATCACATTCATTGCACACAAGATGGATGGAGTCC
AGCAGTACCATGCCTCAGAAAATGTTATTTTCCTTATTTGGAAAATGGATATAAT
CAAAATTATGGAAGAAAGTTTGTACAGGGTAAATCTATAGATGTTGCCTGCCATC
CTGGCTATGCTCTTCCAAAAGCTCAGACCACAGTTACATGTATGGAGAATGGCTG
GTCTCCTACTCCCAGATGCATCAGAGTCAAAACATGTTCCAAATCAAGTATAGAT
ATTGAGAATGGGTTTATTTCTGAATCTCAGTATACATATGCCTTAAAAGAAAAAG
CTAAATATCAATGCAAACTAGGATATGTAACAGCAGATGGTGAAACATCAGGAT
CAATTACATGTGGGAAAGATGGATGGTCAGCTCAACCCACCTGCATTAAATCTAT
AAAAACAGATTGTCTCAGTTTACCTAGCTTTGAAAATGCCATACCCATGGGAGAG
AAGAAGGATGTGTATAAGGCTGGTGAGCAAGTGACTTACACTTGTGCAACATAT
TACAAAATGGATGGAGCCAGTAATGTAACATGCATTAATAGCAGATGGACAGGA
AGGCCAACATGCAGAGACACCTCCTGTGTGAATCCTCCCACAGTACAAAATGCTT
ATATAGTGAGTAGACAGATGAGTAAATATCCATCTGGTGAGAGAGTAAGATATC
AATGTAGGAGCCCTTATGAAATGTTTGGGGATGAAGAAGTGATGTGTTTAAATG
GAAACTGGACAGAACCACCTCAATGCAAAGATTCTACAGGAAAATGTGGGCCCC
CTCCACCTATTGACAATGGGGACATTACTTCATTCCCCTTGTCAGTATATGCTCCA
GCTTCATCAGTTGAGTACCAATGCCAGAACTTGTATCAACTTGAGGGTAACAAGA
GGATAACATGTAGAAATGGACAATGGTCAGAACCACCAAAATGCTTACATCCCT
GTGTAATATCCAGGGAAATTATGGAAAATTATAACATAGCATTAAGGTGGACAG
CCAAACAGAAGCTTTATTCTAGAACAGGTGAATCAGTTGAATTTGTGTGTAAAAG
AGGATATAGACTTTCATCAAGGTCTCACACATTGAGGACAACATGTTGGGATGG
GAAACTGGAGTATCCAACTTGTGCAAAAAGATGA

### ITRs

The inverted terminal repeats (ITR(s)) selected for use in the rAAV virion are preferably AAV sequences, with serotypes 1, 2, 3, 4, 5 and 6 being preferred. The ITRs may be synthetic sequences that function as AAV inverted terminal repeats, such as the “double-D sequence” as described in U.S. Pat. No. 5,478,745 to Samulski et al., the entire disclosure of which is incorporated in its entirety herein by reference. Typically, but not necessarily, the TRs are from the same parvovirus, e.g., both ITR sequences are from AAV2.

In some aspects, the heterologous nucleic encapsulated by the rAAV virion comprises a 5′ ITR with the following sequence:

(SEQ ID NO: 38)
TTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGA
GCGAGCGCGCAGAGAGGGAGTGGCCAACTCCATCACTAGGGGTTCCT

In related aspects, the heterologous nucleic acid encapsulated by the rAAV virion comprises a 3′ ITR with the following sequence:

(SEQ ID NO: 39)
AGGAACCCCTAGTGATGGAGTTGGCCACTCCCTCTCTGCGCGCTCGCTC
GCTCACTGAGGCCGCCCGGGCAAAGCCCGGGCGTCGGGCGACCTTTGGT
CGCCCGGCCTCAGTGAGCGAGCGAGCGCGCAGAGAGGGAGTGGCCAA

### Expression Control Sequences

The hFH gene is operatively linked to at least one transcription control sequence, preferably a transcription control sequence that is heterologous to the nucleic acid. In some aspects, the transcription control sequence comprises a cell- or tissue-specific promoter that results in cell-specific expression of the nucleic acid e.g. in photoreceptor cells, such as human rod photoreceptor-specific human (3-protein coupled receptor rhodopsin kinase 1 (hGRK) promoter or a human interphotoreceptor retinoid-binding protein (IRBP) promoter. In other aspects, the transcription control sequence comprises a constitutive promoter that results in similar expression level of the nucleic acid in many cell types. Suitable constitutive promoters include CAG promoter comprising (C) cytomegalovirus (CMV) immediate-early enhancer element, (A) first exon and the first intron of chicken beta-adin gene and (G) splice acceptor of the rabbit beta-globin gene (see Miyazaki et al. (1989) Gene 79(2): 269-277), cytomegalovirus promoter (CMV) (Stinski et al, (1985) Journal of Virology 55(2): 431-441), human elongation factor l a promoter (ER1a) (Kim et al, (1990) Gene 91(2): 217-223), human phosphoglycerate kinase promoter (POK) (Singer-Sam et al. (1984) Gene 32(3): 409-417), mitochondrial heavy-strand promoter (Loderio et al. (2012) PNAS 109(17): 6513-6518), and ubiquitin promoter (Wulff et al. (1990) FEBS Letters 261: 101-105).

In a preferred aspect, the hfH gene is operably linked to a CAG promoter. In a particularly preferred embodiment, the CAG promoter comprises the sequence of SEQ ID NO:40 or comprises a sequence at least 95%, at least 96%, at least 97%, at least 98% or at least 99% identical thereto:

(SEQ ID NO: 40)
ACTAGTTATTAATAGTAATCAATTACGGGGTCATTAGTTCATAGCCCATATATGG
AGTTCCGCGTTACATAACTTACGGTAAATGGCCCGCCTGGCTGACCGCCCAACGA
CCCCCGCCCATTGACGTCAATAATGACGTATGTTCCCATAGTAACGCCAATAGGG
ACTTTCCATTGACGTCAATGGGTGGAGTATTTACGGTAAACTGCCCACTTGGCAG
TACATCAAGTGTATCATATGCCAAGTACGCCCCCTATTGACGTCAATGACGGTAA
ATGGCCCGCCTGGCATTATGCCCAGTACATGACCTTATGGGACTTTCCTACTTGG
CAGTACATCTACGTATTAGTCATCGCTATTACCATGGTCGAGGTGAGCCCCACGT
TCTGCTTCACTCTCCCCATCTCCCCCCCCTCCCCACCCCCAATTTTGTATTTATTTA
TTTTTTAATTATTTTGTGCAGCGATGGGGGCGGGGGGGGGGGGGGGGCGCGCGC
CAGGCGGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGGCGAGGCG
GCGGCGGCGGCGGCCCTATAAAAAGCGAAGCGCGCGGCGGGGGGGGAGTCGCT
GCGACGCTGCCTTCGCCCCGTGCCCCGCTCCGCCGCCGCCTCGCGCCGCCCGCCC
CGGCTCTGACTGACCGCGTTACTCCCACAGGTGAGCGGGCGGGACGGCCCTTCTC
CTCCGGGCTGTAATTAGCGCTTGGTTTAATGACGGCTTGTTTCTTTTCTGTGGCTG
CGTGAAAGCCTTGAGGGGCTCCGGGAGGGCCCTTTGTGCGGGGGGAGCGGCTCG
GGGGGTGCGTGCGTGTGTGTGTGCGTGGGGAGCGCCGCGTGCGGCTCCGCGCTG
CCCGGCGGCTGTGAGCGCTGCGGGCGCGGCGCGGGGCTTTGTGCGCTCCGCAGT
GTGCGCGAGGGGAGCGCGGCCGGGGGCGGTGCCCCGCGGTGCGGGGGGGGCTG
CGAGGGGAACAAAGGCTGCGTGCGGGGTGTGTGCGTGGGGGGGTGAGCAGGGG
GTGTGGGCGCGTCGGTCGGGCTGCAACCCCCCCTGCACCCCCCTCCCCGAGTTGC
TGAGCACGGCCCGGCTTCGGGTGCGGGGCTCCGTACGGGGCGTGGCGCGGGGCT
CGCCGTGCCGGGCGGGGGGTGGCGGCAGGTGGGGGTGCCGGGGGGGGCGGGGC
CGCCTCGGGCCGGGGAGGGCTCGGGGGAGGGGCGCGGCGGCCCCCGGAGCGCC
GGCGGCTGTCGAGGCGCGGCGAGCCGCAGCCATTGCCTTTTATGGTAATCGTGCG
AGAGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCGGCA
GGAAGGAAATGGGGGGGGAGGGCCTTCGTGCGTCGCCGCGCCGCCGTCCCCTTC
TCCCTCTCCAGCCTCGGGGCTGTCCGCGGGGGGACGGCTGCCTTCGGGGGGGAC
GGGGCAGGGCGGGGTTCGGCTTCTGGCGTGTGACCGGCGGCTCTAGAGCCTCTG
CTAACCATGTTCATGCCTTCTTCTTTTTCCTACAG

In some aspects, the heterologous nucleic acid encapsulated by the rAAV virion comprises a SV40 polyadenylation sequence the following sequence:

(SEQ ID NO: 41)
GGGGATCCAGACATGATAAGATACATTGATGAGTTTGGACAAACCACAA
CTAGAATGCAGTGAAAAAAATGCTTTATTTGTGAAATTTGTGATGCTAT
TGCTTTATTTGTAACCATTATAAGCTGCAATAAACAAGTTAACAACAAC
AATTGCATTCATTTTATGTTTCAGGTTCAGGGGGAGGTGTGGGAGGTTT
TTTAAAGCAAGTAAAACCTCTACAAATGTGGTATGGCTGATTATGATCA

In some aspects, the heterologous nucleic acid comprises the following nucleotide sequence or a nucleotide sequence at least 80%, at least 85%, at least 90%, at least 95%, at least 98% or at least 99% identical thereto:

(SEQ ID NO: 43)
TTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCA
GAGAGGGAGTGGCCAACTCCATCACTAGGGGTTCCTATCGATTGAATTCCCCGG
GGATCCACTAGTTATTAATAGTAATCAATTACGGGGTCATTAGTTCATAGCCCAT
ATATGGAGTTCCGCGTTACATAACTTACGGTAAATGGCCCGCCTGGCTGACCGCC
CAACGACCCCCGCCCATTGACGTCAATAATGACGTATGTTCCCATAGTAACGCCA
ATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGA
CGGTAAATGGCCCGCCTGGCATTATGCCCAGTACATGACCTTATGGGACTTTCCT
ACTTGGCAGTACATCTACGTATTAGTCATCGCTATTACCATGGTCGAGGTGAGCC
CCACGTTCTGCTTCACTCTCCCCATCTCCCCCCCCTCCCCACCCCCAATTTTGTAT
TTATTTATTTITTAATTATTTTGTGCAGCGATGGGGGCGGGGGGGGGGGGGGGGC
GCGCGCCAGGCGGGGCGGGGCGGGGCGAGGGGCGGGGCGGGGCGAGGCGGAG
AGGTGCGGCGGCAGCCAATCAGAGCGGCGCGCTCCGAAAGTTTCCTTTTATGGC
GAGGCGGCGGCGGCGGCGGCCCTATAAAAAGCGAAGCGCGCGGCGGGGGGGGA
GTCGCTGCGACGCTGCCTTCGCCCCGTGCCCCGCTCCGCCGCCGCCTCGCGCCGC
CCGCCCCGGCTCTGACTGACCGCGTTACTCCCACAGGTGAGCGGGGGGACGGC
CCTTCTCCTCCGGGCTGTAATTAGCGCTTGGTTTAATGACGGCTTGTTTCTTTTCT
GTGGCTOCGTGAAAGCCTTGAGGGGCTCCGGGAGGGCCCTTTGTGCGGGGGGAG
CGGCTCGGGGGGTGCGTGCGTGTGTGTGTGCGTGGGGAGCGCCGCGTGCGGCTC
CGCGCTGCCCGGCGGCTGTGAGCGCTGCGGGCGCGGCGCGGGGCTTTGTGCGCT
CCGCAGTGTGCGCGAGGGGAGCGCGGCCGGGGGGGGTGCCCCGCGGTGCGGGG
GGGGCTGCGAGGGGAACAAAGGCTGCGTGCGGGGTGTGTGCGTGGGGGGGTGA
GCAGGGGGTGTGGGCGCGTCGGTCGGGCTGCAACCCCCCCTGCACCCCCCTCCCC
GAGTTGCTGAGCACGGCCCGGCTTCGGGTGCGGGGCTCCGTACGGGGCGTGGCG
CGGGGCTCGCCGIGCCGGGGGGGGGGGGCGGCAGGTGGGGGTGCCGGGCGGG
GCGGGGCCGCCTCGGGCCGGGGAGGGCTCGGGGGAGGGGCGCGGCGGCCCCCG
GAGCGCCGGCGGCTGTCGAGGCGCGGCGAGCCGCAGCCATTGCCTTTTATGGTA
ATCGTGCGAGAGGGCGCAGGGACTTCCTTTGTCCCAAATCTGTGCGGAGCCGAA
ATCTGGGAGGCGCCGCCGCACCCCCTCTAGCGGGCGCGGGGCGAAGCGGTGCGG
CGCCGGCAGGAAGGAAATGGGGGGGGAGGGCCTTCGTGCGTCGCCGCGCCGCCG
TCCCCTTCTCCCTCTCCAGCCTCGGGGCTGTCCGCGGGGGGACGGCTGCCTTCGG
GGGGGACGGGGCAGGGGGGGGTTCGGCTTCTGGCGTGTGACCGGCGGCTCTAGA
GCCTCTGCTAACCATGTTCATGCCTTCTTCTTTTTCCTACAGTCTAGAGTCGACCT
GCAGGTGGATATCTTGCTAGCACGCCACCATGCGCCTGCTGGCCAAGATCATCTG
CCTGATGCTGTGGGCCATCTGCGTGGCCGAGGACTGTAACGAGCTGCCCCCTCGG
AGAAATACAGAGATCCTGACCGGCTCTTGGAGCGATCAGACCTACCCAGAGGGC
ACACAGGCCATCTACAAGTGCAGGCCCGGCTATCGCTCCCTGGGCAATATCATCA
TGGTGTGCAGGAAGGGAGAGTGGGTGGCCCTGAACCCACTGAGAAAGTGCCAGA
AGAGGCCATGTGGACACCCAGGCGACACACCTTTCGGCACCTTTACACTGACCG
GCGGCAACGTGTTCGAGTACGGCGTGAAGGCCGTGTATACCTGCAATGAGGGCT
ACCAGCTGCTGGGCGAGATCAACTACCGGGAGTGTGACACAGATGGCTGGACCA
ATGATATCCCCATCTGCGAGGTGGTGAAGTGTCTGCCAGTGACCGCCCCCGAGA
ACGGCAAGATCGTGAGCTCCGCCATGGAGCCTGACAGAGAGTATCACTTCGGCC
AGGCCGTGAGGTTCGTGTGCAATAGCGGCTACAAGATCGAGGGCGATGAGGAGA
TGCACTGTTCCGACGATGGCTTCTGGTCTAAGGAGAAGCCCAAGTGCGTGGAGA
TCTCCTGTAAGTCTCCTGACGTGATTAACGGCAGCCCAATCTCCCAGAAGATCAT
CTATAAGGAGAATGAGCGGTTTCAGTACAAGTGCAACATGGGCTACGAGTATTC
CGAGAGAGGCGATGCCGTGTGCACAGAGTCTGGATGGAGGCCCCTGCCTAGCTG
CGAGGAGAAGTCCACCCTGAAGCCTTGTGACTATCCAGATATCAAGCACGGCGG
CCTGTATCACGAGAATATGAGGCGCCCTTACTTCCCAGTGGCCGTGGGCAAGTAC
TATTCCTACTATTGCGACGAGCACTTTGAGACACCATCCGGCTCTTACTGGGACC
ACATCCACTGTACCCAGGATGGCTGGTCTCCCGCCGTGCCTTGCCTGAGAAAGTG
TTACTTCCCCTATCTGGAGAACGGCTACAACCAGAATTATGGCAGGAAGTTTGTG
CAGGGCAAGTCCATCGACGTGGCATGCCACCCAGGATACGCACTGCCTAAGGCA
CAGACCACAGTGACCTGTATGGAGAATGGCTGGAGCCCAACACCCAGGTGCATC
CGCGTGAAGACCTGTTCCAAGAGCAGCATCGACATCGAGAACGGCTTCATCAGC
GAGTCCCAGTACACCTATGCCCTGAAGGAGAAGGCCAAGTATCAGTGCAAGCTG
GGATACGTGACCGCAGACGGAGAAACATCTGGCAGCATCACCTGCGGCAAGGAT
GGCTGGTCTGCCCAGCCCACATGCATCAAGAGCATCAAGACCGACTGTCTGTCTC
TGCCCAGCTTTGAGAATGCCATCCCTATGGGCGAGAAGAAGGACGTGTACAAGG
CCGGCGAGCAGGTGACATACACCTGOGCCACATACTATAAGATGGACGGCOCCA
GCAACGTGACCTGTATCAATTCCCGGTGGACAGGCAGACCTACCTGCAGGGATA
CAAGCTGCGTGAACCCACCCACCGTGCAGAATGCCTATATCGTGTCCCGCCAGAT
GTCTAAGTACCCTAGCGGCGAGCGGGTGAGATATCAGTGCCGGTCCCCATACGA
GATGTTCGGCGACGAGGAAGTGATGTGCCTGAACGGCAATTGGACAGAGCCTCC
ACAGTGCAAGGATAGCACCGGCAAGTGTGGACCACCTCCACCAATCGACAACGG
CGATATCACATCCTTTCCACTGAGCGTGTACGCCCCCGCCTCCTCTGTGGAGTAT
CAGTGCCAGAACCTGTACCAGCTGGAGGGCAATAAGCGCATCACCTGTCGGAAC
GGCCAGTGGTCTGAGCCTCCAAAGTGCCTGCACCCTTGCGTGATCTCCAGAGAGA
TCATGGAGAACTATAATATCGCCCTGCGCTGGACAGCCAAGCAGAAGCTGTACT
CTCGGACCGGCGAGAGCGTGGAGTTCGTGTGCAAGCGGGGCTATAGACTGAGCT
CCCGCAGCCACACACTGCGGACCACATGCTGGGACGGCAAGCTGGAGTACCCAA
CCTGTGCCAAGAGGTGAGCCACGCGTAACACGTGCATGCGAGAGATCTGCGGCC
GCGAGCTCGGGGATCCAGACATGATAAGATACATTGATGAGTTTGGACAAACCA
CAACTAGAATGCAGTGAAAAAAATGCTTTATTTGTGAAATTTGTGATGCTATTGC
TTTATTTGTAACCATTATAAGCTGCAATAAACAAGTTAACAACAACAATTGCATT
CATTTTATGTTTCAGGTTCAGGGGGAGGTGTGGGAGGTTTTTTAAAGCAAGTAAA
ACCTCTACAAATGTGGTATGGCTGATTATGATCAATGCATCCTAGCCGGAGGAAC
CCCTAGTGATGGAGTTGGCCACTCCCTCTCTGCGCGCTCGCTCGCTCACTGAGGC
CGCCCGGGCAAAGCCCGGGCGTCGGGCGACCTTTGGTCGCCCGGCCTCAGTGAG
CGAGCGAGCGCGCAGAGAGGGAGTGGCCAA

In related aspects, the heterologous nucleic acid comprises the following nucleotide sequence or a nucleotide sequence at least 80%, at least 85%, at least 90%, at least 95%, at least 98% or at least 99% identical thereto:

(SEQ ID NO: 44)
TTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCA
GAGAGGGAGTGGCCAACTCCATCACTAGGGGTTCCTATCGATTGAACCAGGCGC
GCCCAGTTTAAACAGTCCGGAACTAGTTATTAATAGTAATCAATTACGGGGTCAT
TAGITCATAGCCCATATATGGAGTTCCGCGTTACATAACTTACGGTAAATGGCCC
GCCTGGCTGACCGCCCAACGACCCCCGCCCATTGACGTCAATAATGACGTATGTT
CCCATAGTAACGCCAATAGGGACTTTCCATTGACGTCAATGGGTGGAGTATTTAC
GGTAAACTGCCCACTTGGCAGTACATCAAGTGTATCATATGCCAAGTACGCCCCC
TATTGACGTCAATGACGGTAAATGGCCCGCCTGGCATTATGCCCAGTACATGACC
TTATGGGACTTTCCTACTTGGCAGTACATCTACGTATTAGTCATCGCTATTACCAT
GGTCGAGGTGAGCCCCACGTTCTGCTTCACTCTCCCCATCTCCCCCCCCTCCCCAC
CCCCAATTTTGTATTTATTTATTTTTTAATTATTTTGTGCAGCGATGGGGGCGGGG
GGGGGGGGGGGGCGCGCGCCAGGCGGGGCGGGGCGGGGCGAGGGGCGGGGCG
GGGCGAGGCGGAGAGGTGCGGCGGCAGCCAATCAGAGCGGCGCGCTCCGAAAG
TTTCCTTTTATGGCGAGGCGGCGGCGGCGGCGGCCCTATAAAAAGCGAAGCGCG
CGGCGGGGGGGGAGTCGCTGCGACGCTGCCTTCGCCCCGTGCCCCGCTCCGCCGC
CGCCTCGCGCCGCCCGCCCCGGCTCTGACTGACCGCGTTACTCCCACAGGTGAGC
GGGCGGGACGGCCCTTCTCCTCCGGGCTGTAATTAGCGCTTGGTTTAATGACGGC
TTGTTTCTTTTCTGTGGCTGCGTGAAAGCCTTGAGGGGCTCCGGGAGGGCCCTTT
GTGCGGGGGGAGCGGCTCGGGGGGTGCGTGCGTGTGTGTGTGCGTGGGGAGCGC
CGCGTGCGGCTCCGCGCTGCCCGGCGGCTGTGAGCGCTGCGGGCGCGGCGCGGG
GCTTTGTGCGCTCCGCAGTGTGCGCGAGGGGAGCGCGGCCGGGGGGGGTGCCCC
GCGGTGCGGGGGGGGCTGCGAGGGGAACAAAGGCTGCGTGCGGGGTGTGTGCG
TGGGGGGGTGAGCAGGGGGTGTGGGCGCGTCGGTCGGGCTGCAACCCCCCCTGC
ACCCCCCTCCCCGAGTTGCTGAGCACGGCCCGGCTTCGGGTGCGGGGCTCCGTAC
GGGGCGTGGCGCGGGGCTCGCCGTGCCGGGCGGGGGGTGGCGGCAGGTGGGGG
TGCCGGGCGGGGGGGGGCCGCCTCGGGCCGGGGAGGGCTCGGGGGAGGGGCGC
GGCGGCCCCCGGAGCGCCGGCGGCTGTCGAGGCGCGGCGAGCCGCAGCCATTGC
CTTTTATGGTAATCGTGCGAGAGGGCGCAGGGACTTCCTTTGTCCCAAATCTGTG
CGGAGCCGAAATCTGGGAGGCGCCGCCGCACCCCCTCTAGCGGGCGCGGGGCGA
AGCGGTGCGGCGCCGGCAGGAAGGAAATGGGGGGGGAGGGCCTTCGTGCGTCG
CCGCGCCGCCGTCCCCTTCTCCCTCTCCAGCCTCGGGGCTGTCCGCGGGGGGACG
GCTGCCTTCGGGGGGGACGGGGCAGGGGGGGGTTCGGCTTCTGGCGTGTGACCG
GCGGCTCTAGAGCCTCTGCTAACCATOTTCATGCCTTCTTCTTTTTCCTACAGTCT
AGAGTCGACGOTCACCGGTCAGCTAGCACGCCACCATGCGCCTGCTGGCCAAGA
TCATCTGCCTGATGCTGTGGGCCATCTGCGTGGCCGAGGACTGTAACGAGCTGCC
CCCTCGGAGAAATACAGAGATCCTGACCGGCTCTTGGAGCGATCAGACCTACCC
AGAGGGCACACAGGCCATCTACAAGTGCAGGCCCGGCTATCGCTCCCTGOGCAA
TATCATCATGGTGTGCAGGAAGGGAGAGTGGGTGGCCCTGAACCCACTGAGAAA
GTGCCAGAAGAGGCCATGTGGACACCCAGGCGACACACCTTTCGGCACCITTAC
ACTGACCGGCGGCAACGTGTTCGAGTACGGCGTGAAGGCCGTGTATACCTGCAA
TGAGGGCTACCAGCTGCTGGGCGAGATCAACTACCGGGAGTGTGACACAGATGG
CTGGACCAATGATATCCCCATCTGCGAGGTGGTGAAGTGTCTGCCAGTGACCGCC
CCCGAGAACGGCAAGATCGTGAGCTCCGCCATGGAGCCTGACAGAGAGTATCAC
TTCGGCCAGGCCGTGAGGTTCGTGTGCAATAGCGGCTACAAGATCGAGGGCGAT
GAGGAGATGCACTGTTCCGACGATGGCTTCTGGTCTAAGGAGAAGCCCAAGTGC
GTGGAGATCTCCTGTAAGTCTCCTGACGTGATTAACGGCAGCCCAATCTCCCAGA
AGATCATCTATAAGGAGAATGAGCGGTTTCAGTACAAGTGCAACATGGGCTACG
AGTATTCCGAGAGAGGCGATGCCGTGTGCACAGAGTCTGGATGGAGGCCCCTGC
CTAGCTGCGAGGAGAAGTCCACCCTGAAGCCTTGTGACTATCCAGATATCAAGC
ACGGCGGCCTGTATCACGAGAATATGAGGCGCCCTTACTTCCCAGTGGCCGTGG
GCAAGTACTATTCCTACTATTGCGACGAGCACTTTGAGACACCATCCGGCTCTTA
CTGGGACCACATCCACTGTACCCAGGATGGCTGGTCTCCCGCCGTGCCTTGCCTG
AGAAAGTGTTACTTCCCCTATCTGGAGAACGGCTACAACCAGAATTATGGCAGG
AAGTTTGTCCAGGGCAAGTCCATCGACGTGGCATGCCACCCAGGATACGCACTG
CCTAAGGCACAGACCACAGTGACCTGTATGGAGAATGGCTGGAGCCCAACACCC
AGGTGCATCCGCGTGAAGACCTGTTCCAAGAGCAGCATCGACATCGAGAACGGC
TTCATCAGCGAGTCCCAGTACACCTATGCCCTGAAGGAGAAGGCCAAGTATCAG
TGCAAGCTGGGATACGTGACCGCAGACGGAGAAACATCTGGCAGCATCACCTGC
GGCAAGGATGGCTGGTCTGCCCAGCCCACATGCATCAAGAGCATCAAGACCGAC
TGTCTGTCTCTGCCCAGCTTTGAGAATGCCATCCCTATGGGCGAGAAGAAGGACG
TGTACAAGGCCGGCGAGCAGGTGACATACACCTGCGCCACATACTATAAGATGG
ACGGCGCCAGCAACGTGACCTGTATCAATTCCCGGTGGACAGGCAGACCTACCT
GCAGGGATACAAGCTGCGTGAACCCACCCACCGTGCAGAATGCCTATATCGTGT
CCCGCCAGATGTCTAAGTACCCTAGCGGCGAGCGGGTGAGATATCAGTGCCGGT
CCCCATACGAGATGTTCGGCGACGAGGAAGTGATGTGCCTGAACGGCAATTGGA
CAGAGCCTCCACAGTGCAAGGATAGCACCGGCAAGTGTGGACCACCTCCACCAA
TCGACAACGGCGATATCACATCCTTTCCACTGAGCGTGTACGCCCCCGCCTCCTC
TGTGGAGTATCAGTGCCAGAACCTGTACCAGCTGGAGGGCAATAAGCGCATCAC
CTGTCGGAACGGCCAGTGGTCTGAGCCTCCAAAGTGCCTGCACCCTTGCGTGATC
TCCAGAGAGATCATGGAGAACTATAATATCGCCCTGCGCTGGACAGCCAAGCAG
AAGCTGTACTCTCGGACCGGCGAGAGCGTGGAGTTCGTGTGCAAGCGGGGCTAT
AGACTGAGCTCCCGCAGCCACACACTGCGGACCACATGCTGGGACGGCAAGCTG
GAGTACCCAACCTGTOCCAAGAGGTGAGCCTAGGACTGTACAGTACGOGTAACA
CGTGGGGGATCCAGACATGATAAGATACATTGATGAGTTTGGACAAACCACAAC
TAGAATGCAGTGAAAAAAATGCTTTATTTGTGAAATTTGTGATGCTATTGCTTTA
TTTGTAACCATTATAAGCTGCAATAAACAAGTTAACAACAACAATTGCATTCATT
TTATGTTTCAGGTTCAGGGGGAGGTGTGGGAGGTTTTTTAAAGCAAGTAAAACCT
CTACAAATGTGGTATGGCTGATTATGATCAATGCATGGTCCCAGCCTTCATTGAG
CCTATTGGTCCCCCCTCAGAACCCACCCACCTGGCAGTAGAGGATGTCTCTTGAG
ACACCACTGTCTCCCTCAAGTTCAGGAGGCCCCCATTAATTAAGAGAGAGTGGG
AGCAGGAGGCCTGTCAGATGGCTACTCTTAAGTGGAGTACTGCCCAGAGGGCTG
CTCAGAGTGGGTGGCTGTAACCCTGCAGGGGCTGACAGAGCACACATCCATACT
GGTGAAGGACCTGTGACCCACTGGCCAGACTGGCCGGCCGGAGGAACCCCTAGT
GATGGAGTTGGCCACTCCCTCTCTGCGCGCTCGCTCGCTCACTGAGGCCGCCCGG
GCAAAGCCCGGGCGTCGGGCGACCTTTGGTCGCCCGGCCTCAGTGAGCGAGCGA
GCGCGCAGAGAGGGAGTGGCCAA

In related aspects, the heterologous nucleic acid comprises the following nucleotide sequence or a nucleotide sequence at least 80%, at least 85%, at least 90%, at least 95%, at least 98% or at least 99% identical thereto:

(SEQ ID NO: 45)
TTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCA
GAGAGGGAGTGGCCAACTCCATCACTAGGGGTTCCTATCGATTGAACCAGGCGC
GCCCAGTTTAAACAGTCCGGAACTAGTTATTAATAGTAATCAATTACGGGGTCAT
TAGTTCATAGCCCATATATGGAGTTCCGCGTTACATAACTTACGGTAAATGGCCC
GCCTGGCTGACCGCCCAACGACCCCCGCCCATTGACGTCAATAATGACGTATGTT
CCCATAGTAACGCCAATAGGGACTTTCCATTGACGTCAATGGGTGGAGTATTTAC
GGTAAACTGCCCACTTGGCAGTACATCAAGTGTATCATATGCCAAGTACGCCCCC
TATTGACGTCAATGACGGTAAATGGCCCGCCTGGCATTATGCCCAGTACATGACC
TTATGGGACTTTCCTACTTGGCAGTACATCTACGTATTAGTCATCGCTATTACCAT
GGTCGAGGTGAGCCCCACGTTCTGCTTCACTCTCCCCATCTCCCCCCCCTCCCCAC
CCCCAATTTTGTATTTATTTATTTTTTAATTATTTTGTGCAGCGATGGGGGCGGGG
GGGGGGGGGGGGCGCGCGCCAGGCGGGGCGGGGCGGGGCGAGGGGCGGGGCG
GGGCGAGGCGGAGAGGTGCGGCGGCAGCCAATCAGAGCGGCGCGCTCCGAAAG
TTTCCTTTTATGGCGAGGCGGCGGCGGCGGCGGCCCTATAAAAAGCGAAGCGCG
CGGCGGGGGGGGAGTCGCTGCGACGCTGCCTTCGCCCCGTGCCCCGCTCCGCCGC
CGCCTCGCGCCGCCCGCCCCGGCTCTGACTGACCGCGTTACTCCCACAGGTGAGC
GGGCGGGACGOCCCTTCTCCTCCGGGCTGTAATTAGCGCTTGGTTTAATGACGGC
TTGTTTCTTTTCTGTGGCTGCGTGAAAGCCTTGAGGGGCTCCGGGAGGGCCCTTT
GTGCGGGGGGAGCGGCTCGGGGGGTGCGTGCGTGTGTGTGTGCGTGGGGAGCGC
CGCGTGCGGCTCCGCGCTGCCCGGCGGCTGTGAGCGCTGCGGGCGCGGCGCGGG
GCTTTGTGCGCTCCGCAGTGTGCGCGAGGGGAGCGCGGCCGGGGGGGGTGCCCC
GCGGTGCGGGGGGGGCTGCGAGGGGAACAAAGGCTGCGTGCGGGGTGTGTGCG
TGGGGGGGTGAGCAGGGGGTGTGGGCGCGTCGGTCGGGCTGCAACCCCCCCTGC
ACCCCCCTCCCCGAGTTGCTGAGCACGGCCCGGCTTCGGGTGCGGGGCTCCGTAC
GGGGCGTGGCGCGGGGCTCGCCGTGCCGGGGGGGGGGGGCGGCAGGTGGGGG
TGCCGGGGGGGGGGGGGCCGCCTCGGGCCGGGGAGGGCTCGGGGGAGGGGCGC
GGCGGCCCCCGGAGCGCCGGCGGCTGTCGAGGCGCGGCGAGCCGCAGCCATTGC
CTTTTATGGTAATCGTGCGAGAGGGCGCAGGGACTTCCTTTGTCCCAAATCTGTG
CGGAGCCGAAATCTGGGAGGCGCCGCCGCACCCCCTCTAGCGGGCGCGGGGCGA
AGCGGTGCGGCGCCGGCAGGAAGGAAATGGGCGGGGAGGGCCTTCGTGCGTCG
CCGCGCCGCCGTCCCCTTCTCCCTCTCCAGCCTCGGGGCTGTCCGCGGGGGGACG
GCTGCCTTCGGGGGGGACGGGGCAGGGCGGGGTTCGGCTTCTGGCGTGTGACCG
GCGGCTCTAGAGCCTCTGCTAACCATGTTCATGCCTTCTTCTTTTTCCTACAGTCT
AGAGTCGACGGTCACCGGTCAGCTAGCACGCCACCATGAGGCTGCTGGCCAAGA
TCATCTGCCTGATGCTGTGGGCCATCTGTGTGGCTGAGGACTGTAATGAGCTGCC
CCCTAGGAGAAATACAGAGATCCTGACTGGCTCTTGGTCTGATCAGACCTACCCA
GAGGGCACACAGGCCATCTACAAGTGCAGGCCTGGCTATAGATCCCTGGGCAAT
ATCATCATGGTGTGCAGGAAGGGAGAGTGGGTGGCCCTGAACCCACTGAGAAAG
TGCCAGAAGAGGCCATGTGGACACCCAGGAGACACACCTTTTGGCACCTTTACA
CTGACAGGGGGCAATGTGTTTGAGTATGGTGTGAAGGCAGTGTATACCTGCAAT
GAGGGCTACCAGCTGCTGGGAGAGATCAACTACAGGGAGTGTGACACAGATGGC
TGGACCAATGATATCCCCATCTGTGAGGTGGTGAAGTGTCTGCCAGTGACTGCCC
CTGAGAATGGCAAGATAGTGAGCTCTGCCATGGAGCCTGACAGAGAGTATCACT
TTGGCCAGGCTGTGAGGTTTGTGTGCAATAGTGGCTACAAGATTGAGGGGGATG
AGGAGATGCACTGTTCAGATGATGGCTTCTGGTCTAAGGAGAAGCCCAAGTGTG
TGGAGATCTCCTGTAAGTCTCCTGATGTGATTAATGGCAGCCCAATCTCCCAGAA
GATCATCTATAAGGAGAATGAGAGATTTCAGTACAAGTGCAACATGGGCTATGA
GTATTCAGAGAGAGGTGATGCTGTGTGCACAGAGTCTGGATGGAGGCCCCTGCC
TAGCTGTGAGGAGAAGTCCACCCTGAAGCCTTGTGACTATCCAGATATCAAGCAT
GGAGOCCTGTATCATGAGAATATGAGGAGGCCTTACTTCCCAGTGGCTOTGGGC
AAGTACTATTCCTACTATTGTGATGAGCACTTTGAGACACCATCTGGCTCTTACT
GGGACCACATCCACTGTACCCAGGATGGCTGGTCTCCTGCTGTGCCTTGCCTGAG
AAAGTGTTACTTCCCCTATCTGGAGAATGGCTACAACCAGAATTATGGCAGGAA
GTTTGTGCAGGGCAAGTCCATTGATGTGGCATGCCACCCAGGATATGCACTGCCT
AAGGCACAGACCACAGTGACCTGTATGGAGAATGGCTGGAGCCCAACACCCAGG
TGCATCAGGGTGAAGACCTGTTCCAAGAGCAGCATTGACATAGAGAATGGCTTC
ATCTCAGAGTCCCAGTACACCTATGCCCTGAAGGAGAAGGCCAAGTATCAGTGC
AAGCTGGGATATGTGACAGCAGATGGAGAAACATCTGGCAGCATCACCTGTGGC
AAGGATGGCTGGTCTGCCCAGCCCACATGCATCAAGAGCATCAAGACTGACTGT
CTGTCTCTGCCCAGCTTTGAGAATGCCATCCCTATGGGTGAGAAGAAGGATGTGT
ACAAGGCTGGAGAGCAGGTGACATACACCTGTGCCACATACTATAAGATGGATG
GAGCCAGCAATGTGACCTGTATCAATTCCAGATGGACAGGCAGACCTACCTGCA
GGGATACAAGCTGTGTGAACCCACCCACTGTGCAGAATGCCTATATTGTGTCCAG
GCAGATGTCTAAGTACCCTTCTGGAGAGAGAGTGAGATATCAGTGCAGATCCCC
ATATGAGATGTTTGGGGATGAGGAAGTGATGTGCCTGAATGGCAATTGGACAGA
GCCTCCACAGTGCAAGGATAGCACTGGCAAGTGTGGACCACCTCCACCAATTGA
CAATGGAGATATCACATCCTTTCCACTGTCAGTGTATGCCCCTGCCTCCTCTGTGG
AGTATCAGTGCCAGAACCTGTACCAGCTGGAGGGCAATAAGAGAATCACCTGTA
GAAATGGCCAGTGGTCTGAGCCTCCAAAGTGCCTGCACCCTTGTGTGATCTCCAG
AGAGATCATGGAGAACTATAATATAGCCCTGAGGTGGACAGCCAAGCAGAAGCT
GTACTCTAGGACTGGGGAGTCAGTGGAGTTTGTGTGCAAGAGAGGCTATAGACT
GAGCTCCAGAAGCCACACACTGAGGACCACATGCTOGGATGGCAAGCTGGAGTA
CCCAACCTGTGCCAAGAGGTGAGCCTAGGACTGTACAGTACGCGTAACACGTGG
GGGATCCAGACATGATAAGATACATTGATGAGTTTGGACAAACCACAACTAGAA
TGCAGTGAAAAAAATGCTTTATTTGTGAAATTTGTGATGCTATTGCTTTATTTGTA
ACCATTATAAGCTGCAATAAACAAGTTAACAACAACAATTGCATTCATTTTATGT
TTCAGGTTCAGGGGGAGGTGTGGGAGGTTTTTTAAAGCAAGTAAAACCTCTACA
AATGTGGTATGGCTGATTATGATCAATGCATGGTCCCAGCCTTCATTGAGCCTAT
TGGTCCCCCCTCAGAACCCACCCACCTGGCAGTAGAGGATGTCTCTTGAGACACC
ACTGTCTCCCTCAAGTTCAGGAGGCCCCCATTAATTAAGAGAGAGTGGGAGCAG
GAGGCCTGTCAGATGGCTACTCTTAAGTGGAGTACTGCCCAGAGGGCTGCTCAG
AGTGGGTGGCTGTAACCCTGCAGGGGCTGACAGAGCACACATCCATACTGGTGA
AGGACCTGTGACCCACTGGCCAGACTGGCCGGCCGGAGGAACCCCTAGTGATGG
AGTTGGCCACTCCCTCTCTGCGCGCTCGCTCGCTCACTGAGGCCGCCCGGGCAAA
GCCCGGGCGTCGGGCGACCTTTGGTCGCCCGGCCTCAGTGAGCGAGCGAGCGCG
CAGAGAGGGAGTGGCCAA

In related aspects, the heterologous nucleic acid comprises the following nucleotide sequence or a nucleotide sequence at least 80%, at least 85%, at least 90%, at least 95%, at least 98% or at least 99% identical thereto:

(SEQ ID NO: 46)
TTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCA
GAGAGGGAGTGGCCAACTCCATCACTAGGGGTTCCTATCGATTGAACCAGGCGC
GCCCAGTTTAAACAGTCCGGAACTAGTTATTAATAGTAATCAATTACGGGGTCAT
TAGTTCATAGCCCATATATGGAGTTCCGCGTTACATAACTTACGGTAAATGGCCC
GCCTGGCTGACCGCCCAACGACCCCCGCCCATTGACGTCAATAATGACGTATGTT
CCCATAGTAACGCCAATAGGGACTTTCCATTGACGTCAATGGGTGGAGTATTTAC
GGTAAACTGCCCACTTGGCAGTACATCAAGTGTATCATATGCCAAGTACGCCCCC
TATTGACGTCAATGACGGTAAATGGCCCGCCTGGCATTATGCCCAGTACATGACC
TTATGGGACTTTCCTACTTGGCAGTACATCTACGTATTAGTCATCGCTATTACCAT
GGTCGAGGTGAGCCCCACGTTCTGCTTCACTCTCCCCATCTCCCCCCCCTCCCCAC
CCCCAATTTTGTATTTATTTATTTTTTAATTATTTTGTGCAGCGATGGGGGGGGG
GGGGGGGGGGGGCGCGCGCCAGGCGGGGCGGGGCGGGGCGAGGGGCGGGGCG
GGGCGAGGCGGAGAGGTGCGGCGGCAGCCAATCAGAGCGGCGCGCTCCGAAAG
TTTCCTTTTATGGCGAGGCGGCGGCGGCGGCGGCCCTATAAAAAGCGAAGCGCG
CGGCGGGGGGGGAGTCGCTGCGACGCTGCCTTCGCCCCGTGCCCCGCTCCGCCGC
CGCCTCGCGCCGCCCGCCCCGGCTCTGACTGACCGCGTTACTCCCACAGGTGAGC
GGGCGGGACGGCCCTTCTCCTCCGGGCTGTAATTAGCGCTTGGTTTAATGACGGC
TTGTTTCTTTTCTGTGGCTGCGTGAAAGCCTTGAGGGGCTCCGGGAGGGCCCTTT
GTGCGGGGGGAGCGGCTCGGGGGGTGCGTGCGTGTGTGTGTGCGTGGGGAGCGC
CGCGTGCGGCTCCGCGCTGCCCGGCGGCTGTGAGCGCTGCGGGCGCGGCGCGGG
GCTTTGTGCGCTCCGCAGTGTGCGCGAGGGGAGCGCGGCCGGGGGGGGTGCCCC
GCGGTGCGGGGGGGGCTGCGAGGGGAACAAAGGCTGCGTGCGGGGTGTGTGCG
TGGGGGGGTGAGCAGGGGGTGTGGGCGCGTCGGTCGGGCTGCAACCCCCCCTGC
ACCCCCCTCCCCGAGTTGCTGAGCACGGCCCGGCTTCGGGTGCGGGGCTCCGTAC
GGGGCGTGGCGCGGGGCTCGCCGTGCCGGGCGGGGGGTGGCGGCAGGTGGGGG
TGCCGGGCGGGGGGGGGCCGCCTCGGGCCGGGGAGGGCTCGGGGGAGGGGCGC
GGCGGCCCCCGGAGCGCCGGCGGCTGTCGAGGCGCGGCGAGCCGCAGCCATTGC
CTTTTATGGTAATCGTGCGAGAGGGCGCAGGGACTTCCTTTGTCCCAAATCTGTG
CGGAGCCGAAATCTGGGAGGCGCCGCCGCACCCCCTCTAGCGGGCGCGGGGCGA
AGCGGTGCGGCGCCGGCAGGAAGGAAATGGGGGGGGAGGGCCTTCGTGCGTCG
CCOCGCCGCCOTCCCCTTCTCCCTCTCCAGCCTCGGGGCTGTCCGCGGGGGGACG
GCTGCCTTCGGGGGGGACGGGGCAGGGGGGGGTTCGGCTTCTGGCGTGTGACCG
GCGGCTCTAGAGCCTCTGCTAACCATGTTCATGCCTTCTTCTTTTTCCTACAGTCT
AGAGTCGACGGTCACCGGTCAGCTAGCACGCCACCATGAGACTTCTAGCAAAGA
TTATTTGCCTTATGTTATGGGCTATTTGTGTAGCAGAAGATTGCAATGAACTTCCT
CCAAGAAGAAATACAGAAATTCTGACAGGTTCCTGGTCTGACCAAACATATCCA
GAAGGCACCCAGGCTATCTATAAATGCAGGCCTGGATATAGATCTCTTGGAAAT
ATCATAATGGTATGCAGGAAGGGAGAATGGGTTGCTCTTAATCCATTAAGGAAA
TGTCAGAAAAGGCCCTGTGGACATCCTGGAGATACTCCTTTTGGTACTTTTACCC
TTACAGGAGGAAATGTGTTTGAATATGGTGTAAAAGCTGTGTATACATGTAATGA
GGGGTATCAATTGCTAGGTGAGATTAATTACAGGGAATGTGACACAGATGGATG
GACCAATGATATTCCTATATGTGAAGTTGTGAAGTGTTTACCAGTGACAGCACCA
GAGAATGGAAAAATTGTCAGTAGTGCAATGGAACCAGATAGAGAATACCATTTT
GGACAAGCAGTAAGATTTGTATGTAACTCAGGCTACAAGATTGAAGGAGATGAA
GAAATGCATTGTTCAGATGATGGTTTTTGGAGTAAAGAGAAACCAAAGTGTGTG
GAAATTTCATGCAAATCCCCAGATGTTATAAATGGATCTCCTATATCTCAGAAGA
TTATTTATAAGGAGAATGAAAGATTTCAATATAAATGTAACATGGGTTATGAATA
CAGTGAAAGAGGAGATGCTGTATGCACTGAATCTGGATGGAGACCTTTGCCTTC
ATGTGAAGAAAAATCAACCTTGAAACCTTGTGATTATCCAGACATTAAACATGG
AGGTCTATATCATGAGAATATGAGAAGACCATACTTTCCAGTAGCTGTAGGAAA
ATATTACTCCTATTACTGTGATGAACATTTTGAGACTCCTTCAGGAAGTTACTGG
GATCACATTCATTGCACACAAGATGGATGGAGTCCAGCAGTACCATGCCTCAGA
AAATGTTATTTTCCTTATTTGGAAAATGGATATAATCAAAATTATGGAAGAAAGT
TTGTACAGGGTAAATCTATAGATGTTGCCTGCCATCCTGGCTATGCTCTTCCAAA
AGCTCAGACCACAGTTACATGTATGGAGAATGGCTGGTCTCCTACTCCCAGATGC
ATCAGAGTCAAAACATGTTCCAAATCAAGTATAGATATTGAGAATGGGTTTATTT
CTGAATCTCAGTATACATATGCCTTAAAAGAAAAAGCTAAATATCAATGCAAAC
TAGGATATGTAACAGCAGATGGTGAAACATCAGGATCAATTACATGTGGGAAAG
ATGGATGGTCAGCTCAACCCACCTGCATTAAATCTATAAAAACAGATTGTCTCAG
TTTACCTAGCTTTGAAAATGCCATACCCATGGGAGAGAAGAAGGATGTGTATAA
GGCTGGTGAGCAAGTGACTTACACTTGTGCAACATATTACAAAATGGATGGAGC
CAGTAATGTAACATGCATTAATAGCAGATGGACAGGAAGGCCAACATGCAGAGA
CACCTCCTGTGTGAATCCTCCCACAGTACAAAATGCTTATATAGTGAGTAGACAG
ATGAGTAAATATCCATCTGGTGAGAGAGTAAGATATCAATGTAGGAGCCCTTAT
GAAATGTTTGGGGATGAAGAAGTGATGTGTTTAAATGGAAACTGGACAGAACCA
CCTCAATGCAAAGATTCTACAGGAAAATGTGGGCCCCCTCCACCTATTGACAATG
GGGACATTACTTCATTCCCCTTGTCAGTATATGCTCCAGCTTCATCAGTTGAGTAC
CAATGCCAGAACTTGTATCAACTTGAGGGTAACAAGAGGATAACATGTAGAAAT
GGACAATGGTCAGAACCACCAAAATGCTTACATCCCTGTGTAATATCCAGGGAA
ATTATGGAAAATTATAACATAGCATTAAGGTGGACAGCCAAACAGAAGCTTTAT
TCTAGAACAGGTGAATCAGTTGAATTTGTGTGTAAAAGAGGATATAGACTTTCAT
CAAGGTCTCACACATTGAGGACAACATGTTGGGATGGGAAACTGGAGTATCCAA
CTTGTGCAAAAAGATGAGCCTAGGACTGTACAGTACGCGTAACACGTGGGGGAT
CCAGACATGATAAGATACATTGATGAGTTTGGACAAACCACAACTAGAATGCAG
TGAAAAAAATGCTTTATTTGTGAAATTTGTGATGCTATTGCTTTATTTGTAACCAT
TATAAGCTGCAATAAACAAGTTAACAACAACAATTGCATTCATTTTATGTTTCAG
GTTCAGGGGGAGGTGTGGGAGGTTTTTTAAAGCAAGTAAAACCTCTACAAATGT
GGTATGGCTGATTATGATCAATGCATGGTCCCAGCCTTCATTGAGCCTATTGGTC
CCCCCTCAGAACCCACCCACCTGGCAGTAGAGGATGTCTCTTGAGACACCACTGT
CTCCCTCAAGTTCAGGAGGCCCCCATTAATTAAGAGAGAGTGGGAGCAGGAGGC
CTGTCAGATOGCTACTCTTAAGTGGAGTACTGCCCAGAGGGCTGCTCAGAGTGG
GTGGCTGTAACCCTGCAGGGGCTGACAGAGCACACATCCATACTGGTGAAGGAC
CTGTGACCCACTGGCCAGACTGGCCGGCCGGAGGAACCCCTAGTGATGGAGTTG
GCCACTCCCTCTCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAA

### AAV Capsid

The variant AAV capsid of the rAAV—encapsulating the heterologous nucleic acid encoding an hFh variant—comprises a variant AAV capsid protein comprising an insertion of from about 7 to about 20 amino acids (a “heterologous peptide” or “peptide insertion”) in the GH-loop of a parental AAV capsid protein, wherein the peptide comprises the amino acid sequence ISDQTKH (SEQ ID NO:1). Preferably, the variant capsid protein, when present in an AAV virion, confers increased infectivity of a retinal cell compared to the infectivity of a retinal cell by an AAV virion comprising the corresponding parental capsid protein.

By the “GH loop,” or loop IV, of the AAV capsid protein it is meant the solvent-accessible portion referred to in the art as the OH loop, or loop TV, of AAV capsid protein. For the GH loop/loop IV of AAV capsid, see, e.g., van Viet et al. (2006) Mol. Ther. 14:809; Padron et al. (2005) J. Virol 79:5047; and Shen et al. (2007) Mol. Ther. 15:1955. Thus, for example, the insertion site can be within about amino acids 570-611 of AAV2VP1.

In some embodiments, the peptide insertion has from 1 to 3 spacer amino acids (Y₁-Y₃) at the amino and/or carboxyl terminus of the amino acid sequence ISDQTKH (SEQ ID NO:1). Exemplary spacer amino acids include, without limitation, leucine (1), alanine (A), glycine (G), serine (S), threonine (T), and proline (P). In certain embodiments, a peptide insertion comprises 2 spacer amino acids at the N-terminus and 2 spacer amino acids at the C-terminus. In other embodiments, a peptide insertion comprises 2 spacer amino acids at the N-terminus and 1 spacer amino acids at the C-terminus. In preferred embodiments, the peptide insertion comprises or consists of the amino acid sequence LAISDQTKHA (SEQ ID NO:2).

In some aspects, the variant AAV capsid protein comprises a peptide insertion comprising the amino acid sequence ISDQTKH (SEQ ID NO:1) and further comprises one or more amino acid substitutions relative to a corresponding parental AAV capsid protein. Representative examples of amino acid substitutions may be found at e.g., col. 26, lines 40-65 of U.S. Pat. No. 11,576,983, the entire contents of which are incorporated herein by reference.

In some preferred embodiments, the variant AAV capsid protein comprises a peptide insertion comprising the amino acid sequence ISDQTKH (SEQ ID NO:1) and further comprises a P34A amino acid substitution relative to VP1 capsid of AAV2 or the corresponding substitution in another AAV serotype.

In other aspects, the variant capsid protein may comprise one or more features disclosed in U.S. Pat. No. 11,576,983, in particular, one or more features disclosed at column 26, line 66 to column 29, line 50 of U.S. Patent No. 11,576,983.

In a particularly preferred embodiment, the variant capsid protein comprises the following amino acid sequence or comprises an amino acid sequence at least 80%, at least 90%, least 95%, at least 98%, or at least 99% identical to the following amino acid sequence:

(SEQ ID NO: 42)
MAADGYLPDWLEDTLSEGIRQWWKLKPGPPPPKAAERHKDDSRGLVLPGYKYLGP
FNGLDKGEPVNEADAAALEHDKAYDRQLDSGDNPYLKYNHADAEFQERLKEDTSF
GGNLGRAVFQAKKRVLEPLGLVEEPVKTAPGKKRPVEHSPVEPDSSSGTGKAGQQP
ARKRLNFGQTGDADSVPDPQPLGQPPAAPSGLGTNTMATGSGAPMADNNEGADGV
GNSSGNWHCDSTWMGDRVITTSTRTWALPTYNNHLYKQISSQSGASNDNHYFGYST
PWGYFDFNRFHCHFSPRDWQRLINNNWGFRPKRLNFKLFNIQVKEVTQNDGTTTIA
NNLTSTVQVFTDSEYQLPYVLGSAHQGCLPPFPADVFMVPQYGYLTLNNGSQAVGR
SSFYCLBYFPSQMLRTGNNFTFSYTFEDVPFHSSYAHSQSLDRLMNPLIDQYLYYLSR
TNTPSGTTTQSRLQFSQAGASDIRDQSRNWLPGPCYRQQRVSKTSADNNNSEYSWT
GATKYHLNGRDSLVNPGPAMASHKDDBEKFFPQSGVLIFGKQGSEKTNVDIEKVMIT
DEEEIRTTNPVATEQYGSVSTNLQRGNLAISDQTKHARQAATADVNTQGVLPGMVW
QDRDVYLQGPIWAKIPHTDGHFHPSPLMGGFGLKHPPPQILIKNTPVPANPSTTFSAA
KFASFITQYSTGQVSVEIEWELQKENSKRWNPEIQYTSNYNKSVNVDFTVDINGVYS
EPRPIGTRYLTRNL.

The variant AAV capsid protein of SEQ ID NO:42 contains the following modifications relative to native AAV2 capsid: (i) a proline (P) to alanine (A) mutation at amino acid position 34, which is located inside the assembled capsid (VP1 protein only), and (ii) an insertion of 10 amino acids (leucine-alanine-isoleucine-serine-aspartic acid-glutamine-threonine-lysine-histidine-alanine/LAISDQTKHA (SEQ ID NO:2)) at amino acid position 588, which is present in VP1, VP2, and VP3. In some embodiments, the capsid comprises a variant capsid protein comprising a sequence at least 90%, at least 95%, at least 98%, at least 99% identical to SEQ ID NO:42 and comprising a P34A substitution and an LAISDQTKHA (SEQ ID NO:2) peptide insertion at amino acid position 588.

Also provided herein are packaging cells, which are encompassed by “host cells” which may be cultured to produce packaged viral vectors of the invention. The packaging cells of the invention generally include cells with heterologous (1) viral vector function(s), (2) packaging function(s), and (3) helper function(s). Each of these component functions is discussed in the ensuing sections.

Initially, the vectors can be made by several methods known to skilled artisans (see, e.g., WO 2013/063379). A preferred method is described in Grieger, et al. 2015, Molecular Therapy 24(2):287-297, the contents of which are incorporated by reference herein for all purposes. Briefly, efficient transfection of HEK293 cells is used as a starting point, wherein an adherent HEK293 cell line from a qualified clinical master cell bank is used to grow in animal component-free suspension conditions in shaker flasks and WAVE bioreactors that allow for rapid and scalable rAAV production. Using the triple transfection method (e.g., WO 96/40240), the suspension HEK293 cell line generates greater than 105 vector genome containing particles (vg)/cell or greater than 10′ vg/L of cell culture when harvested 48 hours post-transfection. More specifically, triple transfection refers to the fact that the packaging cell is transfected with three plasmids: one plasmid encodes the AAV rep and cap genes, another plasmid encodes various helper functions (e.g., adenovirus or HSV proteins such as E1a, B1b, E2a, E4, and VA RNA, and another plasmid encodes the transgene and its various control elements (e.g., modified GLA gene and CAG promoter).

To achieve the desired yields, a number of variables are optimized such as selection of a compatible serum-free suspension media that supports both growth and transfection, selection of a transfection reagent, transfection conditions and cell density. A universal purification strategy, based on ion exchange chromatography methods, was also developed that resulted in high purity vector preps of AAV serotypes 1-6, 8, 9 and various chimeric capsids. This user-friendly process can be completed within one week, results in high full to empty particle ratios (>90% full particles), provides post-purification yields (>1×10\hat{ }13 vg/L) and purity suitable for clinical applications and is universal with respect to all serotypes and chimeric particles. This scalable manufacturing technology has been utilized to manufacture GMP Phase I clinical AAV vectors for retinal neovascularization (AAV2), Hemophilia B (scAAVS), Giant Axonal Neuropathy (scAAV9) and Retinitis Pigmentosa (AAV2), which have been administered into patients. In addition, a minimum of a 5-fold increase in overall vector production by implementing a perfusion method that entails harvesting rAAV from the culture media at numerous time-points post-transfection.

The packaging cells include viral vector functions, along with packaging and vector functions. The viral vector functions typically include a portion of a parvovirus genome, such as an AAV genome, with rep and cap deleted and replaced by the modified GLA sequence and its associated expression control sequences. The viral vector functions include sufficient expression control sequences to result in replication of the viral vector for packaging. Typically, the viral vector includes a portion of a parvovirus genome, such as an AAV genome with rep and cap deleted and replaced by the transgene and its associated expression control sequences. The transgene is typically flanked by two AAV TRs, in place of the deleted viral rep and cap ORFs. Appropriate expression control sequences are included, such as a tissue-specific promoter and other regulatory sequences suitable for use in facilitating tissue-specific expression of the transgene in the target cell. The transgene is typically a nucleic acid sequence that can be expressed to produce a therapeutic polypeptide or a marker polypeptide.

The terminal repeats (TR(s)) (resolvable and non-resolvable) selected for use in the viral vectors are preferably AAV sequences, with serotypes 1, 2, 3, 4, 5 and 6 being preferred. Resolvable AAV TRs need not have a wild-type TR sequence (e.g., a wild-type sequence may be altered by insertion, deletion, truncation or missense mutations), as long as the TR mediates the desired functions, e.g., virus packaging, integration, and/or provirus rescue, and the like. The TRs may be synthetic sequences that function as AAV inverted terminal repeats, such as the “double-D sequence” as described in U.S. Pat. No. 5,478,745 to Samulski et al, the entire disclosure of which is incorporated in its entirety herein by reference. Typically, but not necessarily, the TRs are from the same parvovirus, e.g., both TR sequences are from AAV2.

The packaging functions include variant capsid components as described above.

The packaged viral vector includes a variant hfH transgene and expression control sequences flanked by TR elements, referred to herein as the “transgene” or “transgene expression cassette,” sufficient to result in packaging of the vector DNA and subsequent expression of the gene sequence in the transduced cell. The viral vector functions may, for example, be supplied to the cell as a component of a plasmid or an amplicon. The viral vector functions may exist extrachromosomally within the cell line and/or may be integrated into the cell's chromosomal DNA.

Any method of introducing the nucleotide sequence carrying the viral vector functions into a cellular host for replication and packaging may be employed, including but not limited to, electroporation, calcium phosphate precipitation, microinjection, cationic or anionic liposomes, and liposomes in combination with a nuclear localization signal. In embodiments wherein the viral vector functions are provided by transfection using a virus vector; standard methods for producing viral infection may be used.

The packaging functions include genes for viral vector replication and packaging. Thus, for example, the packaging functions may include, as needed, functions necessary for viral gene expression, viral vector replication, rescue of the viral vector from the integrated state, viral gene expression, and packaging of the viral vector into a viral particle. The packaging functions may be supplied together or separately to the packaging cell using a genetic construct such as a plasmid or an amplicon, a Baculovirus, or HSV helper construct the packaging functions may exist extrachromosomally within the packaging cell, but are preferably integrated into the cell's chromosomal DNA. Examples include genes encoding AAV Rep and Cap proteins.

The helper functions include helper virus elements needed for establishing active infection of the packaging cell, which is required to initiate packaging of the viral vector. Examples include functions derived from adenovirus, baculovirus and/or herpes virus sufficient to result in packaging of the viral vector. For example, adenovirus helper functions will typically include adenovirus components E1a, E1b, E2a, E4, and VA RNA. The packaging functions may be supplied by infection of the packaging cell with the required virus. The packaging functions may be supplied together or separately to the packaging cell using a genetic construct such as a plasmid or an amplicon. See, e.g., pXR helper plasmids as described in Rabinowitz eat., 2002, J. Virol. 76:791, and pDG plasmids described in Grimm et at., 1998, Human Gene Therapy 9:2745-2760. The packaging functions may exist extrachromosomally within the packaging cell, but are preferably integrated into the cell's chromosomal DNA (e.g., E1 or E3 in HEK 293 cells).

Any suitable helper virus functions may be employed. For example, where the packaging cells are insect cells, baculovirus may serve as a helper virus. Herpes virus may also be used as a helper virus in AAV packaging methods. Hybrid herpes viruses encoding the AAV Rep protein(s) may advantageously facilitate for more scalable AAV vector production schemes.

Any method of introducing the nucleotide sequence carrying the helper functions into a cellular host for replication and packaging may be employed, including but not limited to, electroporation, calcium phosphate precipitation, microinjection, cationic or anionic liposomes, and liposomes in combination with a nuclear localization signal. In embodiments wherein the helper functions are provided by transfection using a virus vector or infection using a helper virus; standard methods for producing viral infection may be used.

Any suitable permissive or packaging cell known in the art may be employed in the production of the packaged viral vector. Mammalian cells or insect cells are preferred. Examples of cells useful for the production of packaging cells in the practice of tie invention include, for example, human cell lines, such as VERO, W138, MRC5, A549, HEK 293 cells (which express functional adenoviral E1 under the control of a constitutive promoter), B-50 or any other HeLa cells, HepG2, Saos-2, HuH7, and HT1080 cell lines. In one aspect, the packaging cell is capable of growing in suspension culture, more preferably, the cell is capable of growing in serum-free culture. In one embodiment, the packaging cell is a HEK293 that grows in suspension in serum free medium. In another embodiment, the packaging cell is the HEK293 cell described in U.S. Pat. No. 9,441,206 and deposited as ATCC No. PTA 13274. Numerous rAAV packaging cell lines are known in the art, including, but not limited to, those disclosed in WO 2002/46359. In another aspect, the packaging cell is cultured in the form of a cell stack (e.g. 10-layer cell stack seeded with HEK293 cells).

Cell lines for use as packaging cells include insect cell lines. Any insect cell which allows for replication of AAV and which can be maintained in culture can be used in accordance with the present invention, Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, N J (1995); O'Reilly et at, Baculovirus Expression Vectors: A Laboratory Manual, Oxford Univ. Press (1994); Samulski et al., 1989, J Virol. 63:3822-3828; Kajigaya et al., 1991, Proc. Nat't. Acad. Sci. USA 88: 4646-4650; Ruffing et al., 1992, 3. Virol. 66:6922-6930; Kimbauer et at, 1996, Virol. 219:37-44; Zhao et al., 2000, Virol. 272:382-393; and Samulski et al., U.S. Pat. No. 6,204,059,

Virus capsids according to the invention can be produced using any method known in the art, e.g., by expression from a baculovirus (Brown et at, (1994) Virology 198:477-488). As a further alternative, the virus vectors of the invention can be produced in insect cells using baculovirus vectors to deliver the rep/cap genes and rAAV template as described, for example, by Urabe et al., 2002, Human Gene Therapy 13:1935-1943.

In another aspect, the present invention provides for a method of rAAV production in insect cells wherein a baculovirus packaging system or vectors may be constructed to carry the AAV Rep and Cap coding region by engineering these genes into the polyhedrin coding region of a baculovirus vector and producing viral recombinants by transfection into a host cell. Notably when using Baculovirus production for AAV, preferably the AAV DNA vector product is a self-complementary AAV like molecule without using mutation to the AAV ITR. This appears to be a by-product of inefficient AAV rep nicking in insect cells which results in a self-complementary DNA molecule by virtue of lack of functional Rep enzyme activity. The host cell is a baculovirus-infected cell or has introduced therein additional nucleic acid encoding baculovirus helper functions or includes these baculovirus helper functions therein. These baculovirus viruses can express the AAV components and subsequently facilitate the production of the capsids.

During production, the packaging cells generally include one or more viral vector functions along with helper functions and packaging functions sufficient to result in replication and packaging of the viral vector. These various functions may be supplied together or separately to the packaging cell using a genetic construct such as a plasmid or an amplicon, and they may exist extrachromosomally within the cell line or integrated into the cell's chromosomes.

The cells may be supplied with any one or more of the stated functions already incorporated, e.g., a cell line with one or more vector functions incorporated extrachromosomally or integrated into the cell's chromosomal DNA, a cell line with one or more packaging functions incorporated extrachrornosomally or integrated into the cell's chromosomal DNA, or a cell line with helper functions incorporated extrachromosomally or integrated into the cell's chromosomal DNA.

The rAAV vector may be purified by methods standard in the art such as by column chromatography or cesium chloride gradients. Methods for purifying rAAV vectors are known in the art and include methods described in Clark et al., 1999, Human Gene Therapy 10(6):1031-1039; Schenpp and Clark, 2002, Methods Mol. Med. 69:427-443; U.S. Pat. No. 6,566,118 and WO 98/09657.

### Methods of Delivering Nucleic Acids Encoding hfH to the Retina

In several embodiments, methods for delivering heterologous nucleotide sequences encoding hfH to the retina are provided utilizing an rAAV as herein described. The rAAV may be employed to deliver a nucleotide sequence encoding hfH to a retinal cell in vitro, e.g., to produce an hfH polypeptide or nucleic acid in vitro for ex vivo gene therapy. The rAAV are additionally useful in a method of delivering a nucleotide sequence to a subject in need thereof, e.g., to express hfH in a subject in need thereof, such as human with dry AMD or a human with geographic atrophy. In this manner, the hfH may thus be produced in vivo in the subject to restore complement regulation.

Thus, in one aspect, a method of delivering a nucleic acid encoding an hfH variant to a retinal cell is provided, the method comprising contacting the retinal cell with an rAAV virion as herein described.

In another aspect, a method of delivering a nucleic acid encoding an hfH variant to a retinal cell in a mammalian subject is provided, the method comprising administering an effective amount of the rAAV virion as herein described or a pharmaceutical formulation comprising same to a mammalian subject.

The rAAV may be administered to retina of a subject by any suitable route. In preferred embodiments, the rAAV is administered to the subject intraocularly, preferably by subretinal, suprachoroidal, and/or intravitreal injection. In some particularly preferred embodiments, rAAV is administered to a subject via intravitreal injection, more preferably by a single intravitreal injection.

### Treatment Methods

In certain embodiments, a method is provided for the treatment of dry AMD in a subject in need of such treatment, the method comprising administering to the subject a recombinant adeno-associated virus (rAAV) comprising: (a) a variant AAV capsid protein comprising a heterologous peptide insertion with a length of 7 to 20 amino acids covalently inserted in the GH-loop of the AAV capsid protein, wherein the peptide insertion comprises the amino acid sequence ISDQTKH (SEQ ID NO: 1) and (ii) a heterologous nucleic acid comprising a nucleotide sequence encoding an hfH f variant as herein described, said nucleotide sequence operably linked to a promoter, or administering to the subject a pharmaceutical composition comprising said rAAV and a pharmaceutically acceptable carrier, preferably wherein said rAAV or said pharmaceutical composition is administered to the subject by intravitreal injection. Also provided is the use of said rAAV or a pharmaceutical composition comprising same for the treatment of dry AMD. Also provided is the use of said rAAV in the manufacture of a medicament for the treatment of dry AMD.

In related embodiments, a method is provided for the treatment of a geographic atrophy in a subject in need of such treatment, the method comprising administering to the subject a recombinant adeno-associated virus (rAAV) virion comprising: (a) a variant AAV capsid protein comprising a heterologous peptide insertion with a length of 7 to 20 amino acids covalently inserted in the GH-loop of the AAV capsid protein, wherein the peptide insertion comprises the amino acid sequence ISDQTKH (SEQ ID NO:1) and (ii) a heterologous nucleic acid comprising a nucleotide sequence encoding an hfH variant as herein described, said nucleotide sequence operably linked to a promoter, or administering to the subject a pharmaceutical composition comprising said rAAV virion and a pharmaceutically acceptable carrier, preferably wherein said rAAV or said pharmaceutical composition is administered to the subject by intravitreal injection. Also provided is the use of said rAAV or a pharmaceutical composition comprising same for the treatment of geographic atrophy. Also provided is the use of said rAAV in the manufacture of a medicament for the treatment of geographic atrophy.

In some aspects, the variant AAV capsid protein comprises an amino acid sequence at least 80%, at least 85%, at least 90%, at least 95%, at least 98%, at least 99% identical to, or is 100% identical to, the amino acid sequence set forth as SEQ ID NO:42 and comprises a P34A substitution and an LAISDQTKIA (SEQ ID NO:2) peptide insertion at amino acid position 588.

In some preferred aspects, the nucleotide sequence encoding the hfH variant encodes an htH variant with the structure SCR1-(L1)-SCR2-(L2)-SCR3-(L3)-SCR4-(L4)-SCR6-(L5)-SCR7-(L6)-SCR8-(L7)-SCR17-(L8)-SCR18-(L9)-SCR19-(L0)-SCR20, wherein each of SCRs 1-4, 6-8 and 17-20 comprises the amino acid sequence according to Table 1 and wherein each of L1-L10 comprises the amino acid sequence according to Table 2. In related aspects, the nucleotide sequence encoding the hfH variant encodes an hfH variant comprising the amino acid sequence of SEQ ID NO:34 or comprising an amino acid sequence at least 90%, at least 95%, at least 98%, or at least 99% identical to the amino acid sequence set forth as SEQ ID NO:34. In other related aspects, the nucleotide sequence encoding the hfH variant comprises the nucleotide sequence set forth as any one of SEQ ID Nos: 35-37.

In some aspects, the rAAV is administered by periocular, intravitreal, suprachoroidal and/or subretinal injection, preferably by intravitreal injection, to a subject with dry AMD and/or geographic atrophy at a dose of from about 1×10⁸ vector genomes (vg)/eye to about 1×10¹³ vg/eye, from about 1×10⁸ vg/eye to about 1×10¹² vg/eye, from about 1×10⁹ vg/eye to about 1×10¹² vg/eye, from about 1×10⁹ vg/eye to about 1×10¹¹ vg/eye or from about 6×10⁹ vg/eye to about 6×10¹⁰ vg/eye. In some embodiments, the rAAV is administered by periocular, intravitreal, suprachoroidal and/or subretinal injection, preferably by intravitreal injection, to a subject with dry AMD and/or geographic atrophy at a dose of about 1×10⁸ vg/eye, about 2×10⁸ vg/eye, about 3×10⁸ vg/eye, about 4×10⁸ vg/eye, about 5×10⁸ vg/eye, about 6×10⁸ vg/eye, about 7×10⁸ vg/eye, about 8×10⁸ vg/eye, about ×10⁸ vg/eye, about 1×10⁹ vg/eye, 2×10⁹ vg/eye, about 3×10⁹ vg/eye, about 4×10⁹ vg/eye, about 5×10⁹ vg/eye, about 6×10¹⁰ vg/eye, about 7×10⁹ vg/eye, about 8×10⁹ vg/eye, about 9×10⁹ vg/eye, about 1×10¹⁰ vg/eye, about 2×10¹⁰ vg/eye, about 3×10¹⁰ vg/eye, about 4×10¹⁰ vg/eye, about 5×10¹⁰ vg/eye, about 6×10¹¹ vg/eye, about 7×10¹⁰ vg/eye, about 8×10¹⁰ vg/eye, about 9×10¹⁰ vg/eye, about 1×10¹¹ vg/eye, about 2×10¹¹ vg/eye, about 3×10¹¹ vg/eye, about 4×10¹¹ vg/eye, about 5×10¹¹ vg/eye, about 6×10¹¹ vg/eye, about 7×10¹¹ vg/eye, about 8×10¹¹ vg/eye, about 9×10¹¹ vg/eye or about 1×10¹² vg/eye.

In some embodiments, a method for treating dry AMD and/or geographic atrophy in a subject in need thereof is provided comprising administering to the subject by periocular, intravitreal, suprachoroidal and/or subretinal injection, an effective amount of an rAAV virion comprising (i) a capsid comprising a capsid protein comprising the amino acid sequence set forth as SEQ ID NO:42 and (ii) a heterologous nucleic acid comprising from 5′ to 3′: (a) an AAV2 terminal repeat (b) a CAG or CMV promoter (c) a nucleotide sequence selected from SEQ ID Nos:35-37 (d) a polyadenylation sequence and (e) an AAV2 terminal repeat.

In related embodiments, a method for treating dry AMD and/or geographic atrophy in a subject in need thereof is provided comprising administering to the subject by periocular, intravitreal, suprachoroidal and/or subretinal injection, a pharmaceutical composition comprising a pharmaceutically acceptable carrier and an rAAV virion comprising (i) a capsid protein comprising the amino acid sequence set forth as SEQ ID NO:42 and (ii) a heterologous nucleic acid comprising from 5′ to 3′: (a) an AAV2 terminal repeat (b) a CAG or CMV promoter (c) a nucleotide sequence selected from SEQ ID Nos:35-37 (d) a polyadenylation sequence and (e) an AAV2 terminal repeat. In some aspects, the pharmaceutical composition comprises between about 1×10⁴ vg to about 1×10¹³ vg, between about 1×10⁹ vg to about 1×10¹² vg, between about 1×10⁹ vg to about 1×10¹¹ vg or between about 6×10⁹ vg to about 6×10¹⁰ vg. In otter aspects, the pharmaceutical composition comprises about 1×10⁸ vg, about 2×10⁸ vg, about 3×10⁸ vg, about 4×10⁸ vg, about 5×10⁸ vg, about 6×10⁸ vg, about 7×10⁸ vg, about 8×10⁸ vg, about ×10⁸ vg, about 1×10⁹ vg, 2×10⁹ vg, about 3×10⁹ vg, about 4×10⁹ vg, about 5×10⁹ vg, about 6×10⁹ vg, about 7×10⁹ vg, about 8×10⁹ vg, about 9×10⁹ vg, about 1×10¹⁰ vg, about 2×10¹⁰ vg, about 3×10¹⁰ vg, about 4×10¹⁰ vg, about 5×10¹⁰ vg, about 6×10¹⁰ vg, about 7×10¹⁰ vg, about 8×10¹⁰ vg, about 9×10¹⁰ vg, about 1×10¹¹ vg, about 2×10¹¹ vg, about 3×10¹¹ vg, about 4×10¹¹ vg, about 5×10¹¹ vg, about 6×10¹¹ vg, about 7×10¹¹ vg, about 8×10¹¹ vg, about 9×10¹¹ vg or about 1×10′¹² vg.

Pharmaceutical compositions comprising an rAAV as described herein are provided. In some embodiments, the pharmaceutical composition comprises about 1×10⁸ to about 1×10¹⁴ vector particles or vector genomes, about 1×10⁸ to about 1×10¹³ vector particles or vector genomes, about 1×10⁹ to about 1×10¹² vector particles or vector genomes, or about 1×10⁸, about 2×10⁸, about 3×10⁸, about 4×10⁸, about 5×10⁸, about 6×10⁸, about 7×10⁸, about 8×10⁸, about 9×10⁸, about 1×10⁹, about 2×10⁹, about 3×10⁹, about 4×10⁹, about 5×10⁹, about 6×10⁹ about 7×10⁹, about 8×10⁹, about 9×10⁹, about 1×10¹⁰, about 2×10¹⁰, about 3×10¹⁰, about 4×10¹⁰, about 5×10¹⁰, about 6×10¹⁰, about 7×10¹⁰, about 8×10¹⁰, about 9×10¹⁰, about 1×10¹¹, about 2×10¹¹, about 3×10¹¹, about 4×10¹¹, about 5×10¹¹, about 6×10¹¹, about 7×10¹¹, about 8×10¹¹, about 9×10¹¹ or about 1×10¹² vector particles or vector genomes. In some aspects, the pharmaceutical composition comprises about 1×10⁹ to about 1×10¹¹ vg, and preferably comprises about 6×10⁹ vg to about 6×10¹⁰ vg. In some preferred embodiments, the pharmaceutical composition is administered to a human with dry AMD and/or geographic atrophy via intravitreal injection.

### EXAMPLES

The following examples illustrate preferred embodiments of the present invention and are not intended to limit the scope of the invention in any way. While this invention has been described in relation to its preferred embodiments, various modifications thereof will be apparent to one skilled in the art from reading this application.

### Example 1

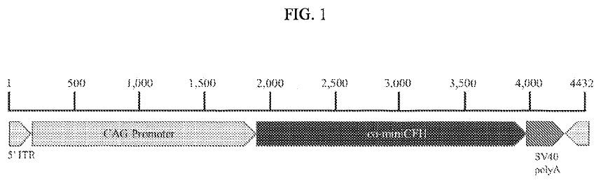

A recombinant AAV (rAAV) virion was constructed carrying a transgene encoding a shortened form of human complement factor H (FIG. 1). Complement factor H (CFH) is a natural inhibitor of the alternative complement pathway and as such the rAAV is useful for the treatment of, inter alia, geographic atrophy secondary to age-related macular degeneration. Endogenous CFH is a 155 kD protein comprising 20 short consensus repeat (SCR) units. The N-terminus is the critical site for co-factor activity/decay accelerating activity and the C-terminus for cell regulation (see e.g., de Córdoba S R, de Jorge E G. Translational mini-review series on complement factor H: genetics and disease associations of human complement factor H. Clin Exp Immunol. 2008 January;151(1):1-13).

The rAAV comprises a capsid protein comprising the amino acid sequence set forth in SEQ ID NO:42 and a heterologous nucleic acid encoding a shortened from of complement factor H (“miniCFH”) having the amino acid sequence set forth in SEQ ID NO:34, operably linked to a CAG promoter. The miniCEFH transgene contains SCRs1-4, 6-8 and 17-20.

The rAAV was subjected to in vitro characterization by (a) transfection of the rAAV plasmid cassette in human HEK293T cells and (b) in vitro transduction of human iPSC-derived retinal pigment epithelial (RIE) cells with the rAAV.

### Expression and Activity of MiniCFH in HEK293T Cells

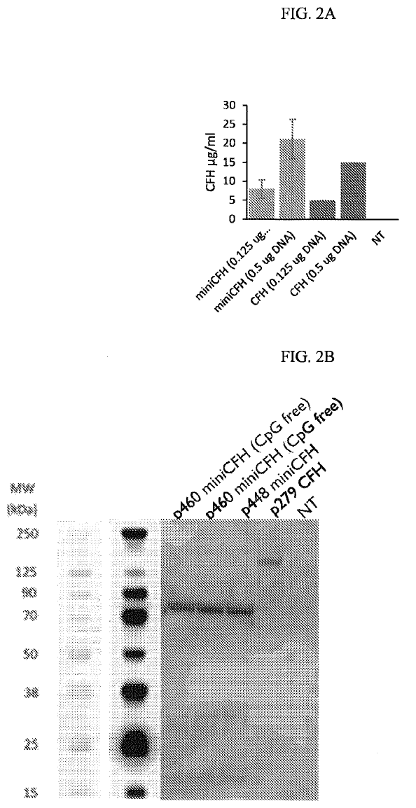

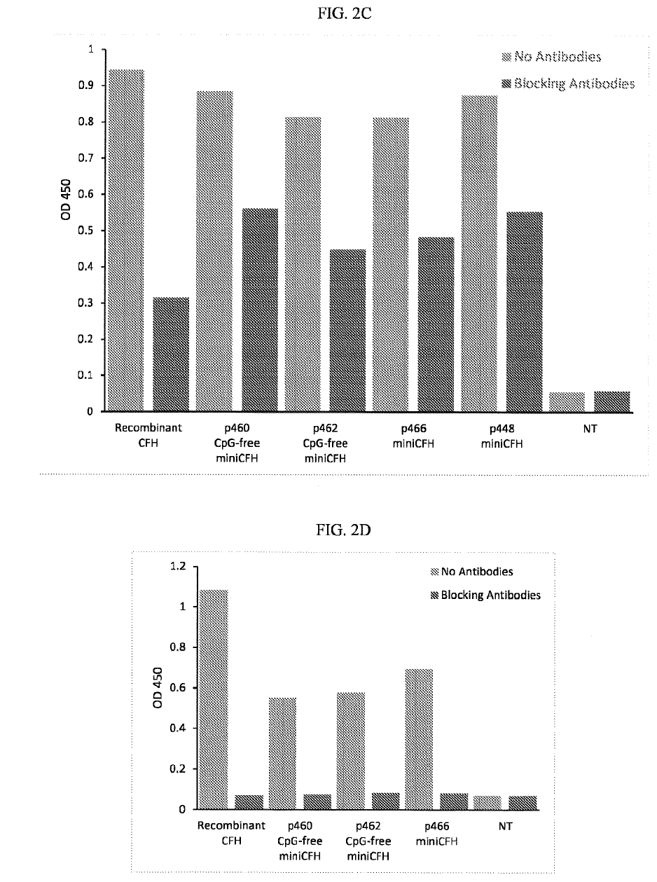

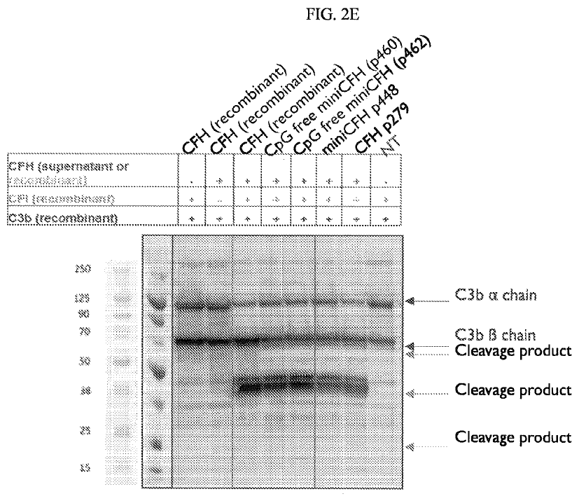

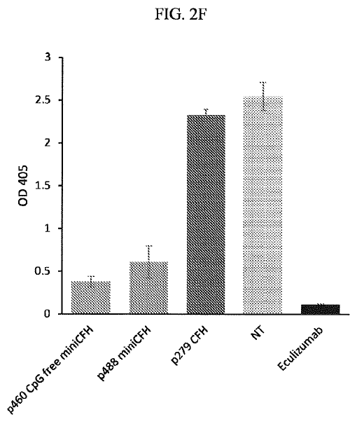

To demonstrate in vitro function of the miniCFH transgene utilized in the rAAV, human cells were transfected with the rAAV plasmid cassette. HEK293T cells were transfected at low (0.125 μg) or high (0.5 g) plasmid DNA encoding a miniCFH or CFH (full length), each driven by a CAG promoter. Dose-dependent expression and properly sized protein was observed from cell supernatants (FIGS. 2A and 2B), miniCFH from cell supernatants was shown to bind directly to 3b and heparin similar to full length CFH (FIGS. 2C and 2D). These data confirm that the miniCFH transgene is expressed from the rAAV plasmid cassette, secreted, and retains complement and heparin binding characteristics. Next, the activity of the miniCFH transgene was assessed and functionality was confirmed. The complement 3 (C3) protein is cleaved into C3a and C3b fragments and the C3b fragment further degraded by complement factor I (CPI), in the presence of CFH as a co-factor, into smaller inactive fragments to interrupt the alternative complement cascade. Protein lysates from rAAV plasmid cassette-transfected cells demonstrate that miniCFH mediated C3b degradation (FIG. 2E). Further, miniCFH from transfected cells demonstrated complete MAC formation inhibition (FIG. 2F). In total, these results demonstrate that transfection with miniCFH cDNA leads to expression and secretion of functional CFH protein.

In Vitro Transduction of Human iPSC-Derived Retinal Pigment Epithelial (RPE) Cells

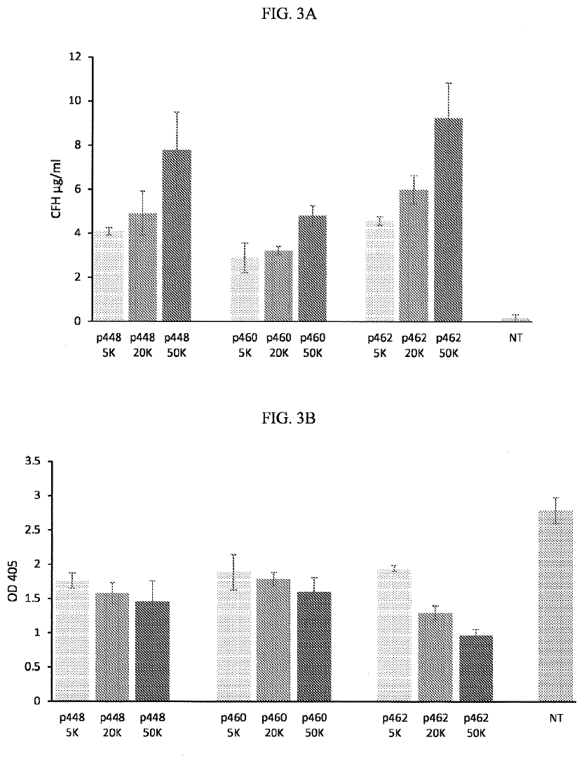

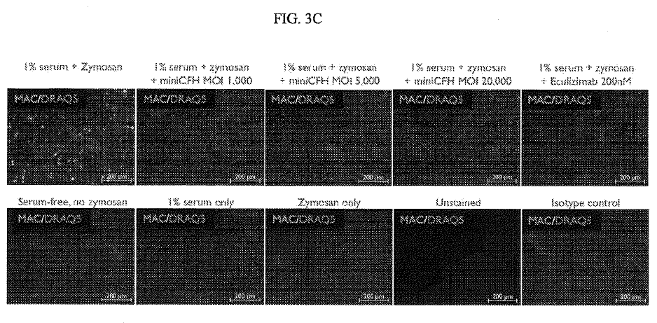

To demonstrate in vitro expression and confirm function of the miniCFH transgene following transduction with the rAAV, human iPSC-derived RPE cells were transduced with the rAAV at different multiplicities of infection (MOI). Transduction of cells with the rAAV resulted in dose-dependent secreted protein expression of the miniCFH transgene (FIG. 3A). Additionally, the alternative complement pathway was inhibited demonstrated MAC formation inhibition (FIGS. 3B and 3C). Together these in vitro data confirm expression, secretion, and functionality of the miniCFH transgene from the rAAV.

### Materials and Methods

Expression of miniCFH (ELISA, WB). HEK293T cells (CRL-3216 from ATCC) were transfected with plasmid DNA encoding a miniCFH or CFH (full length) driven by CAG promoter. Plasmid DNA was transfected at 0.125 μg or 0.5 g DNA/12 well) (2e5 cells/well) with Fugene HD reagent (Promega Corporation cat #E2311). Cells were incubated for 48 h in 1 ml DMEM media with 1% Penicillin/Streptomycin and 10% heat-inactivated serum. After the initial 48h, the media was exchanged for 0.35 ml serum free media (DMEM media with 1% Penicillin/Streptomycin) and cells were incubated for 24h. Serum-free supernatants were tested for transgene expression by ELISA and Western blot.

C3b cleavage assay was performed to confirm the functional activity of CFH expressed in 293T cells. C3 protein is made of alpha and beta chains. The C3 protein is cleaved into C3a and C3b fragments. C3a is a small, 77 amino acids, fragment with approximate molecular weight of 9 kDa. C3b fragment (containing the remaining alpha and beta chains) can form C3 convertase upon binding factor B, or can be degraded by complement factor I (CFH), in the presence of CFH as a co-factor, into smaller inactive fragments. The later event interrupts the alternative complement cascade. These smaller fragments resulting from the C3b degradation can be detected by Western blot. Serum free media supernatant from CFH-transfected cells (or un-transfected cells at negative control) was incubated with 1 μg purified C3b (Complement Technology Inc. cat #A114) and 1 g purified CFI (Complement Technology Inc. cat #A138) for 1 hour at 37° C. As a positive control we used 0.5 μg purified CFH. (Complement Technology Inc. cat #A137) mixed with 1 μg purified C3b (Complement Technology Inc. cat #A114) and 1 μg purified CFI (Complement Technology Inc. cat #A138) in PBS and incubated for 1 hour at 37° C. Additional negative controls where either CFH or CFI are omitted in the mix of recombinant proteins were included.

Complement inhibition assay. The Wieslab® Complement system Alternative pathway kit (Eagle Biosciences Inc. cat #COMPL AP 330) is an enzyme immunoassay for the qualitative determination of functional alternative complement pathway in human serum. The assay combines principles of the hemolytic assay for complement activation with the use of labeled antibodies specific for neoantigen produced as a result of complement activation. The amount of neoantigen generated is proportional to the functional activity of complement pathways. The wells of the 96 well plate are coated with specific activator of the alternative pathway (Bacterial lipopolysaccharides, LPS). Normal human serum is diluted to 5% in diluent containing specific blocker to ensure that only the alternative pathway is activated. During the incubation of the diluted serum in the wells, complement is activated by the LPS. The wells are then washed, and C5b-9 is detected with a specific alkaline phosphatase labelled antibody to the neoantigen expressed during MAC formation. After a further washing step, detection of specific antibodies is obtained by incubation with alkaline phosphatase substrate solution. The amount of complement activation correlates with the color intensity and is measured in terms of absorbance (optical density (OD). Addition of complement inhibitors such as Eculizumab (a C5-blocking antibody), CFH from CFH-transfected cell culture supernatants, or CFH recombinant protein to the 5% normal human serum can inhibit the reaction partially or fully as evidenced by a decreased absorbance signal.

C3b binding ELISA. The C3b binding ELISA is an in-house ELISA adapted from Nichols et. al. (2/pb.ned.ncbjhil.niigov/26221-73A The assay tests if CpG-free miniCFH retains direct C3b binding ability by utilizing monoclonal anti-CFH blocking antibodies to prevent miniCFH binding to a C3b coated plate. The assay was carried out with both full-length recombinant CFH as proof of concept, and supernatants from 293T cells transfected with CpG-free miniCFH. As a control, we included conditions of mock incubation of CFH and transfected cell supernatant with no antibodies.

Heparin binding ELISA. The Heparin binding ELISA is an in-house ELISA adapted from Nichols et. al. (//pubmed.ne info.nih.gov/2622 7534. The assay tests if CpG-free miniCFH retains heparin binding ability by utilizing monoclonal anti-CFH blocking antibodies to prevent miniCFH binding to a heparin coated plate. The assay was carried out with both full-length recombinant CFH as a control, and with supernatants from 293T cells transfected with CpG-free miniCFH. As controls, we also included transfected cell supernatant not treated with blocking antibodies. Results show that preincubation with blocking CFH antibodies reduces recombinant CFH and CpG-free miniCFH signal, indicating that CpG-free miniCFH constructs bind retain heparin binding activity.

Transduction of human RPE cells. IPSC-derived RPE were transduced with a capsid of SEQ ID NO:42 delivering a payload encoding miniCFH driven by a CAG promoter. RPE were previously derived and cryopreserved in-house. 30+/−5 days prior to transduction, RPE were seeded onto plates coated with hESC-qualified Matrigel™ (Coming cat #354277) and maintained in serum-free X-VIVO-10 media (Lonza cat #BEBP02-055Q) with 1% Penicillin/Streptomycin and RHO/LOCK pathway inhibitor Y-27632 (STEMCELL Technologies cat #72304) at 10 uM 30+/−5 days. Media was changed every 2-3 days. RPE were transduced at 5,000; 20,000; and 50,000 multiplicity of infection (MOI, vector genomes (vg) per cell). Cell counts were performed on the same day and just prior to transductions; the appropriate volume of virus was mixed with fresh media and added to cells. After the initial 3 days following transduction, media was removed, and fresh media added. 4 days later, 7 days post-transduction, media was harvested and assayed for miniCFH expression and activity.

High performance liquid chromatography/mass spectrometry (LC-MS) was employed to confirm the presence of the miniCFI gene product (short CFH or sCFH) in non-human primates (NWPs) following intravitreal administration of the rAAV of Example 1 (comprising a capsid protein comprising the amino acid sequence set forth in SEQ ID NO:42 and a heterologous nucleic acid encoding a shortened from of complement factor H (“miniCFH”) having the amino acid sequence set forth in SEQ ID NO:34, operably linked to a CAG promoter).

### Materials and Methods

Aqueous humor (AH) and Vitreous Humor (VH) samples were collected from N-IPs treated (intravitreally) with the rAAV, snap frozen and stored for analysis. sCF-LCMS method was developed & qualified to identify peptide signatures specific to the sCFM encoded by the rAAV. These specific peptide mass signatures were then quantified against a standard curve to determine the concentration of SCFH protein in each sample. Quality control samples containing known levels of sCFH are included in each experimental run. Standard deviation acceptance criteria for repeated sample testing is $20%. LLOQ/BLQ for assay was determined to be—50 ng/mL and samples were run in triplicate.

### Results

Table 3 below contains a summary of the results of LCMS quantification of sCFH concentrations in AH samples of NHPs intravitreally administered the specified dose (1e10, 5e10, 5e11 vg/eye) of rAAV (see also FIR. 4):

TABLE 3

| sample | dose | timepoint | avg mini-CFH | | BLQ |
|---|---|---|---|---|---|
| type | (vg/eye) | (days) | ng/mL | SD | frequency |
| AH | 1e10 | 15 | 58.27 | 34.80 | 0/6 BLQ |
| AH | 1e10 | 29 | 52.83 | 22.74 | 0/6 BLQ |
| AH | 1e10 | 43 | 66.16 | 34.83 | 1/6 BLQ |
| AH | 1e10 | 58 | 94.00 | 0.85 | 4/6 BLQ |
| AH | 1e10 | 71 | 87.30 | 3.25 | 4/6 BLQ |
| AH | 1e10 | 85 | 85.10 | 25.31 | 4/6 BLQ |
| AH | 5e10 | 15 | 104.40 | 48.37 | 0/8 BLQ |
| AH | 5e10 | 29 | 164.62 | 139.38 | 2/8 BLQ |
| AH | 5e10 | 43 | 65.53 | 19.63 | 2/6 BLQ |
| AH | 5e10 | 58 | 43.30 | 5.37 | 2/4 BLQ |
| AH | 5e10 | 71 | 39.95 | 0.78 | 2/4 BLQ |
| AH | 5e10 | 85 | 44.50 | 4.53 | 2/4 BLQ |
| AH | 5e11 | 15 | 176.95 | 87.40 | 0/6 BLQ |
| AH | 5e11 | 29 | 118.53 | 104.92 | 0/6 BLQ |
| AH | 5e11 | 43 | 107.02 | 97.77 | 0/6 BLQ |
| AH | 5e11 | 58 | 146.00 | 63.64 | 0/2 BLQ |
| AH | 5e11 | 71 | 85.15 | 2.19 | 0/2 BLQ |
| AH | 5e11 | 85 | 39.30 | 1.27 | 0/2 BLQ |

Table 4, below, contains a summary of the results of LCMS quantification of sCFH concentrations in VH samples of NHPs treated with the specified dose of rAAV:

TABLE 4

| sample | dose | timepoint | avg mini-CFH | | BLQ |
|---|---|---|---|---|---|
| type | (vg/eye) | (days) | ng/mL | SD | frequency |
| VH | 1e10 | 85 | 213.00 | NA | 2/3 BLQ |
| VH | 5e10 | 43 | 309.00 | NA | 1/2 BLQ |
| VH | 5e10 | 85 | 141.00 | NA | 1/2 BLQ |
| VH | 5e11 | 42 | 120.57 | 52.97 | 0/3 BLQ |
| VH | 5e11 | 86 | 80.10 | NA | 0/1 BLQ |

### Conclusion

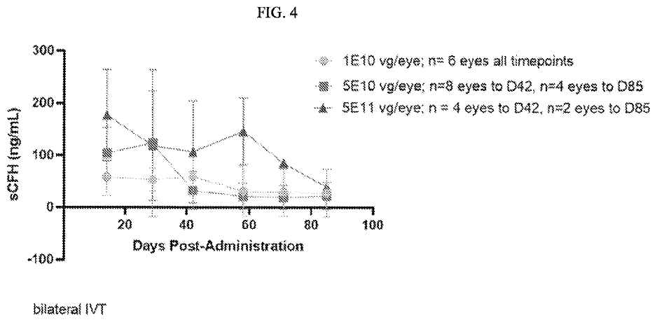

sCFH gene product encoded by the rAAV was detected in VH and AH at each timepoint and dose (Tables 3 and 4; FIG. 4). Based on Ali levels of sCFH measured and previous studies/models with rAAV comprising a capsid protein of SEQ ID NO:42 exploring relative concentrations of AH/VH/retina, retinal sCFH concentrations were predicted to be within normal/therapeutic range (see Table 5 below)

TABLE 5

| | | Predicted | Predicted | | | |
|---|---|---|---|---|---|---|
| | | Peak AH | Peak AH | | | |
| | | conc | conc | | | |
| | | (ng/ml) | (ng/ml) | | | |
| | Mean | (All PK | (Low PK | Predicted | Predicted | |
| | Observed | Regres- | Regres- | R/C conc | R/C sCFH | |
| Dose | AH | sion) | sion) | (ng/g) | conc (nM) | |
| 1e12 | — | 373 | 2232 | 15,624 | 195 | nM |
| 5e11 | 1767 (87) | 187 | 1116 | 13,606 | 170 | nM |
| 1E11 | — | 37 | 223 | 1719 | 21.5 | nM |
| 5e10 | 10 (48) | 19 | 112 | 800 | 10 | nM |
| 3.3e10 | — | 12.3 | 74 | 570 | 7 | nM |
| 1e10 | 58 (35) | 3.7 | 22.3 | 447 | 5.6 | nM |
| 6e9 | — | 2.2 | 13 | 103 | 1.29 | nM |
| 3e9 | — | 1 | 6.7 | 52 | 0.65 | nM |
| 1e9 | — | 0.4 | 2.2 | 17 | 0.213 | nM |

Endogenous CFH AH: 50-350 ng/ml
Endogenous CFH predicted retina/choroid: 3-70 nM

An in situ hybridization (ISH) method was developed to assess expression of sCFH using transfected 293T cells to confirm specificity and optimize assay conditions. The ISH method was then used to detect the presence of sCFH RNA. in NHP ocular tissue following intravitreal injection with the rAAV (comprising a capsid protein comprising the amino acid sequence set forth in SEQ ID NO:42 and a heterologous nucleic acid encoding miniCFH having the amino acid sequence set forth in SEQ ID NO:34, operably linked to a CAG promoter).

### Materials and Methods

Ocular tissue samples (whole globe eye sample) were collected from NHPs treated with the rAAV at 5E10 vg/eye, fixed and processed for analysis.

The sCFH ISH assay was performed using a custom, proprietary 20ZZ probe to sCFH mRNA (designed to not cross react to endogenous cyno or human FL CFH) targeting amino acids 195-1318 (sCFH ISH was performed on the Roche Discovery ULTRA Autostainer platform using Roche DISC. mRNA Probe AMP. Kit RUO, Roche mRNA Sample Prep Kit RUO, DISC. mRNA DAB Detection RUO, Bluing Reagent and Hematoxylin and ACD RNAscope® VS Universal HRP Reagent Kit. Whole slide image scans were captured using Zeiss Axioscan instrument and representative images were captured and reported using Zeiss Zen-lite imaging software.

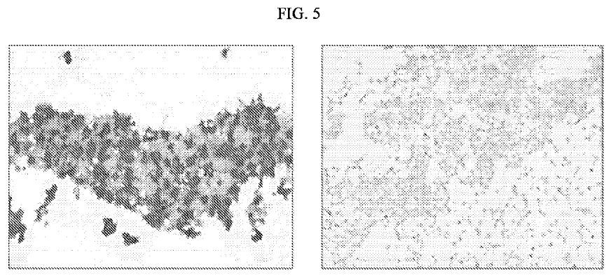

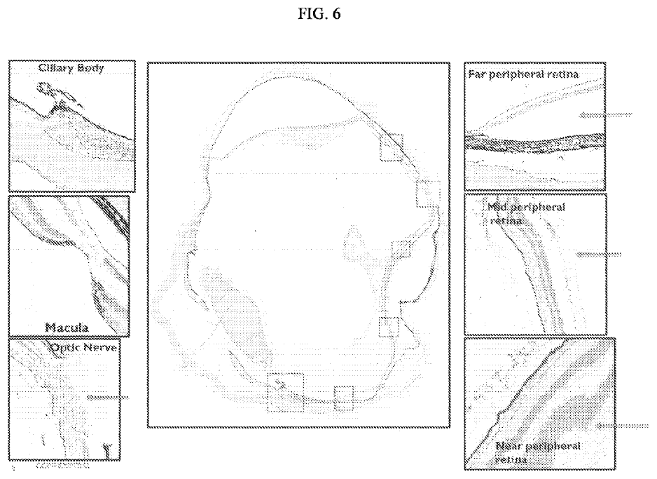

The rIsults of the ISH analysis are shown in FIGS. 5 and 6. FIG. 5 demonstrates sCFH RNA in transfected HEK293T cells compared to untransfected (negative control) HEK293T cells. FIG. 6 demonstrates widespread sCFH RNA iSH signal in the retina and macula regions.

### Example 3

Additional in vitro studies were performed to demonstrate the in vitro functional activity of the expressed miniCFH protein following transfection of human HEK293T cells.

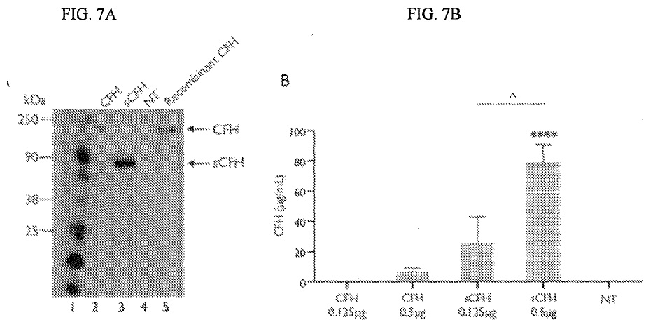

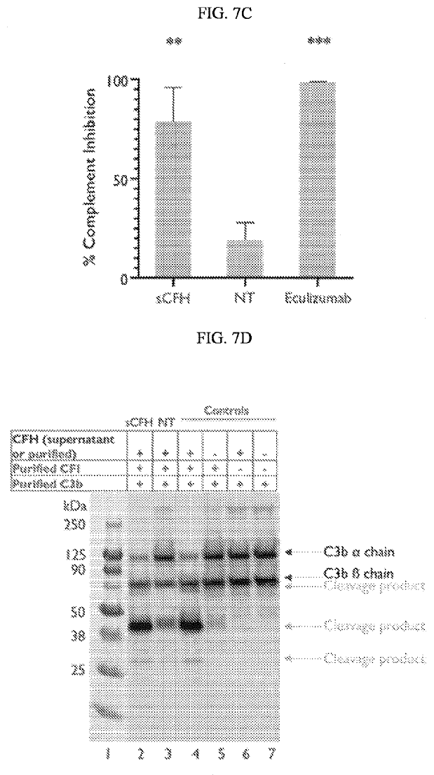

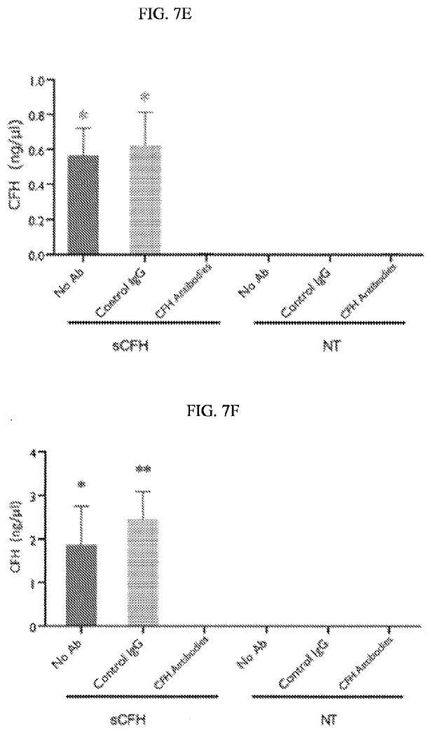

HEK293T cells were transfected with a low (0.125 μg) or high level (0.5 μg) of the rAAV plasmid cassette of Example 1 (comprising a heterologous nucleic acid encoding a shortened from of complement factor H (“miniCFH”) having the amino acid sequence set forth in SEQ ID NO:34, operably linked to a CAG promoter) or an rAAV plasmid cassette encoding full-length CFH protein. The miniCFH or full length CFH protein secreted into the supernatant was visualized by Western blot. Dose-dependent expression and properly sized protein was observed in supernatants from transfected cells (FIG. 7A). Secreted CFH protein expression was quantified following transfections with the rAAV plasmid cassette of Example 1 by ELISA (FIG. 7B). Transfection led to secreted and detectable CFH expression over non-transfection control (FIG. 7B).

In addition to protein expression, activity of miniCFH secreted from HEK293T cells transfected with the miniCFH construct was evaluated. Complement factor H is a complement regulator essential for controlling the alternative pathway and, in turn, negatively regulates the formation of the C5b-9 complex, also called the Membrane Attack Complex (MAC), an endpoint of the complement cascade. To test whether miniCFH was functional, alternative complement inhibiting activity was measured using a modified version of the Wieslab® Complement System Alternative Pathway Assay. Supematants from cells transfected with the rAAV plasmid cassette of Example 1 showed significant inhibition of alternative complement system activity (FIG. 7C). Recombinant eculizumab antibody, a C5 inhibitor, was used as a positive control and shows strong inhibition of the alternative complement pathway in this assay (FIG. 7C).

In addition to assaying the effect of miniCFH on the endpoint of complement activation, MAC formation, an upstream mechanism of CFH was evaluated via the C3b cleavage activity assay. An upstream complement protein C3 can be hydrolyzed into C3a and C3b fragments upon complement activation. The C3b fragment contains an alpha and beta chain and can either form C3 convertase upon binding factor B or can be degraded by complement factor I (CFI), in the presence of CFH as a co-factor, into smaller inactive cleavage products. The degradation of C3b beta chain interrupts the alternative complement cascade and is a marker of complement pathway inhibition. These smaller fragments resulting from the C3b cleavage can be detected by Western blot. Supernatant from cells transfected with the rAAV plasmid cassette of Example 1 or non-transfected cells were incubated with recombinant proteins CFI and C3b and analyzed by Western blot (FIG. 7D). The membrane was probed with an anti-C3 antibody to detect all C3 protein products including C3 alpha chain, C3 beta chain and three beta chain cleavage products. In the reaction incubated with supernatants of cells transfected with the rAAV plasmid cassette of Example 1, all three C3b cleavage products can be seen, similar to positive control but not detected in NT reaction. Additionally, the C3b a chain band was depleted in this condition similar to the positive control. These data support the upstream function of miniCFH produced from the rAAV plasmid cassette of Example 1 to aid CF in the cleavage of C3b beta chain to ultimately inhibit the progression of the complement cascade.

Furthermore, as expected, miniCFH from cell supematants of cells transfected with the rAAV plasmid cassette of Example 1 bound directly to both C3b and heparin similarly to full length CFH (FIG. 7E and FIG. 7F). These data confirmed that the miniCFH transgene could be expressed, secreted, and retained proper complement and heparin binding characteristics.

Further experiments were performed to demonstrate expression of the miniCFH gene product as well as functional activity of the expressed miniCFH protein in vector-transduced human iPSC-derived RPE cells following transduction at different multiplicities of infection (MOI). iPSC-derived RPE cells were transduced with rAAV at several different MOI: 5,000; 20,000; and 50,000 vg/cell for experiments to assess the expression and functional activity of miniCFH secreted into cell supernatants, and MOI 800; 2,000; and 5,000 vg/cell for disease model experiments. The rAAV comprise a capsid protein comprising the amino acid sequence set forth in SEQ ID NO:42 and a heterologous nucleic acid encoding a shortened from of complement factor H (“miniCFH”) having the amino acid sequence set forth in SEQ ID NQ:34, operably linked to a CAG promoter. Following transduction, the viral inoculum was removed after approximately 72 hours and media was changed. Seven days post transduction, cell supernatants were collected, aliquoted, and stored at −80° C. and cells were fixed.

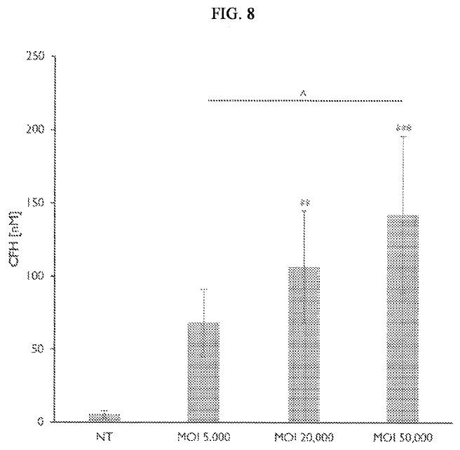

In order to demonstrate that transduction of iPSC-derived RPE cells with the rAAV results in expression of a functional protein, the concentration of secreted miniCFH in the cell supernatants was quantified by ELISA seven days post-transduction. The ELISA detects both endogenous full-length CFH and the miniCFH encoded by the rAAV. Signal beyond that seen from non-transduced cells correlates to miniCFH expressed from the rAAV. As shown in FIG. 8, non-transduced RPE cells secrete low levels of endogenous CFH, and supernatants from RPEs transduced at MOIs of 20,000 and 50,000 showed statistically higher CFH concentrations. Moreover, the CFH concentration was significantly higher in supernatants from RPE cells transduced at MOI 50,000 compared to MOI 5,000. This data demonstrates an in vitro dose-dependent response.

Subsequently, the ability of the secreted miniCFH protein to inhibit the alternative complement pathway was also assessed in RPE cells transduced with rAAV comprising a capsid protein comprising the amino acid sequence set forth in SEQ ID NO:42 and a heterologous nucleic acid encoding the amino acid sequence set forth in SEQ ID NO:34, operably linked to a CAG promoter. Alternative complement inhibiting activity was measured in supernatants from transduced cells via two different methods. 1) A modified version of the Wieslab® Complement System Alternative Pathway assay and 2) Quantification of soluble C5b-9. In order to assess the functional inhibitory effect of miniCFH on Wieslab® Complement System Alternative pathway, supernatant samples from RPE tells transduced with the rAAV were mixed with serum and samples were applied to the Wieslab assay plates. Complement pathway inhibition resulted in a decreased MAC signal.

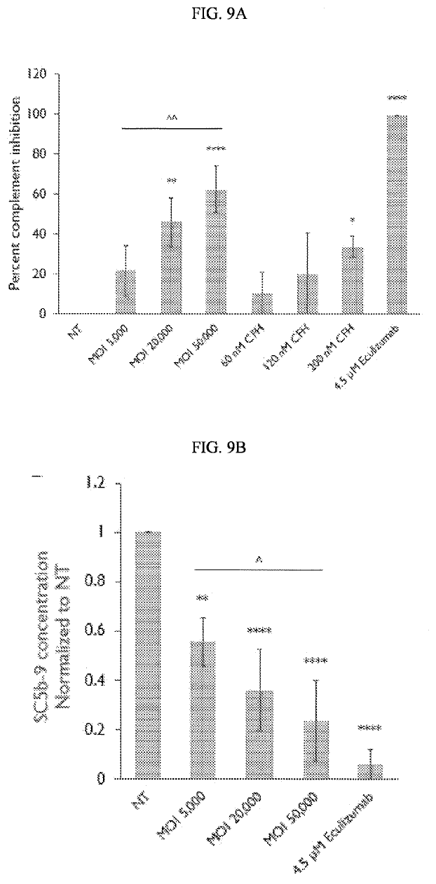

Supernatants from cells transduced with the rAAV at 20,000 MOI and 50,000 MOI produced statistically significant complement inhibition relative to the non-transduced RPE cell samples. As a positive control, RPE cells were treated with 200 nM recombinant CFH, a concentration comparable to the highest levels of expressed miniCFH seen in this study. Dose-dependent complement inhibitory activity was observed, and treatment of RPE cells with a high concentration eculizumab, a known complement inhibitor that targets C5, also resulted in near-complete complement inhibition. (FIG. 9A)

Since the Wieslab assay is not a truly quantitative measure of complement activation, the complement-inhibitory activity of expressed miniCFH was evaluated using a second, more quantitative method. The concentration of soluble C5b-9 was measured using a SC5b ELISA kit. Supernatant samples from RPE transduced with the rAAV showed significantly decreased SC5b-9 formation in comparison to samples with supernatant from non-transduced RPE (FIG. 9B). Furthermore, a dose-dependent effect on soluble C5b-9 formation was also observed. As expected, non-transduced cell samples supplemented with eculizumab showed minimal C5b-9 formation. This decrease in soluble C5b-9 demonstrates functional inhibition of the alternative complement pathway. Together, these results demonstrated that miniCFH secreted by RPE cells transduced with the rAAV was able to inhibit the alternative complement pathway in a dose-dependent manner.

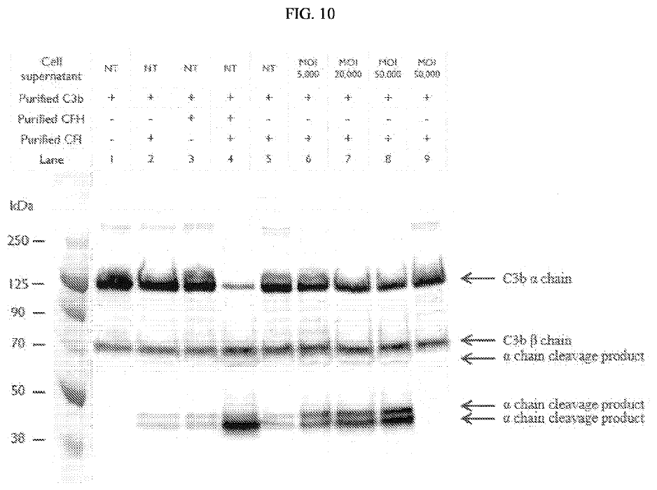

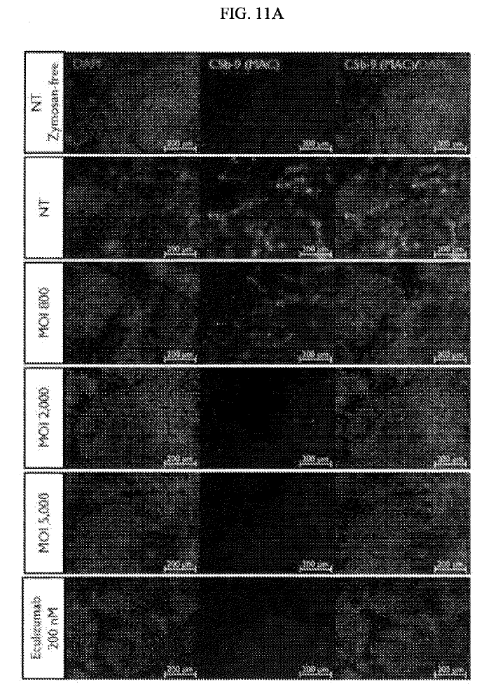

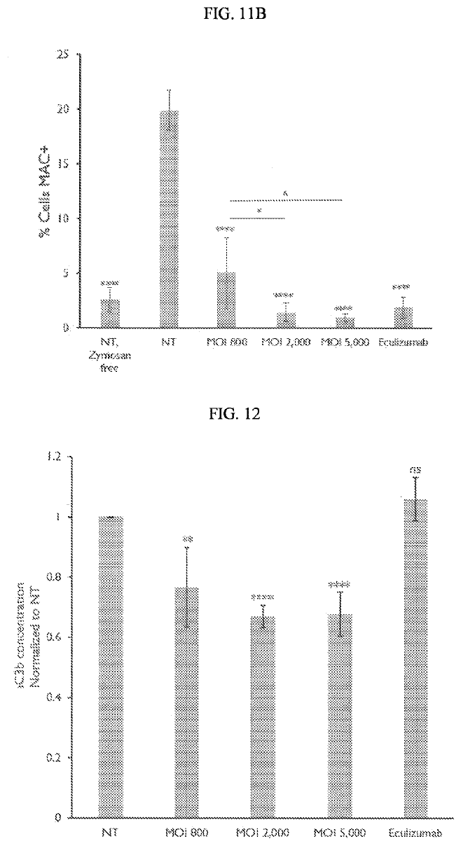

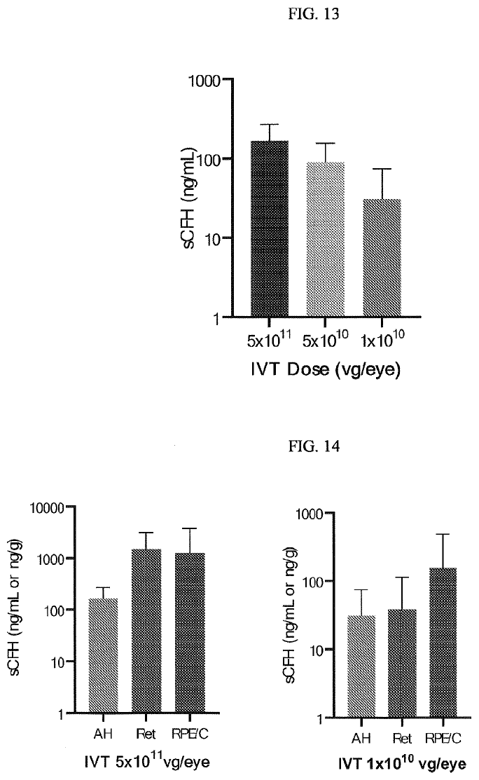

In addition to characterizing the functional effect of the expressed miniCFH: on complement activation, an upstream mechanism of miniCFH was evaluated via a C3 convertase decay acceleration activity assay. In order to determine whether miniCFH acts as a cofactor with Complement Factor I (CFI) to promote C3b breakdown in the same manner as full-length CFH, supernatants from cells transduced with the rAAV were combined with purified C3b and CFI in place of purified full-length CFH (FIG. 10, lanes 5-9). Compared to non-transduced samples (FIG. 10, lane 5), samples from transduced RPE (FIG. 10, lanes 6-8) resulted in greater C3b a chain cleavage, with the higher MOI samples producing more cleavage than the lower MOI samples. In the absence of CI, the miniCFH produced by RPE produced minimal cleavage of C3b, similar to purified full length CFH (FIG. 10, lane 9), supporting its role as a cofactor in the breakdown of C3b.

In addition to these findings, the efficacy of rAAV comprising a capsid protein comprising the amino acid sequence set forth in SEQ TD NO:42 and a heterologous nucleic acid encoding the amino acid sequence set forth in SEQ ID NO:34, operably linked to a CAG promoter, was examined in an in vitro human RPE disease model. For this purpose, a model of retinal complement activation was established in human iPSC-derived RPE cultures. To mimic the disease state, serum and the alternative complement pathway activator Zymosan were introduced to the culture medium, resulting in the deposition of MAC on the RPE over the following 24 hours, as determined by Immunocytochemistry (ICC) and flow cytometry.

As shown below in FIG. 11, rAAV treatment of RPE. six days prior to complement activation elicited a dose-dependent decrease in MAC deposition on RPE triggered by the presence of Zymosan. Eculizumab, when added to cultures simultaneously with complement activation, exerted a similar protective effect, as expected. Flow cytometric quantification of MAC deposition (FIG. 11B) revealed that RPE cells transduced with the rAAV and RPE supplemented with Eculizumab exhibited significantly decreased MAC formation in comparison to non-transduced RPE. Moreover, MAC formation was significantly lower in RPE cells transduced at MOI 2,000 and 5,000 compared to MOI 800, indicating a dose-dependent response. Together, these immunocytochemistry (ICC) and flow cytometry data demonstrated that miniCFH secreted from RPE transduced with the rAAV product, in addition to their inhibition of complement in cell-free assays, also protected RPE from MAC deposition in a disease-like context.

Finally, the upstream function of miniCFH1 in this in vitro model was assessed. The activation of the alternative complement pathway results in a large increase in C3b, which is converted to iC3b. As discussed earlier, CFH is a cofactor in the cleavage of C3b to iC3b; however, because this cleavage inactivates the C3 convertase, the creation of iC3b ultimately results in less C3b being produced and therefore less iC3b in the system, iC3b is commonly used as a readout for C3b, because it has a significantly greater half-life compared with C3b. Therefore, iC3b concentration was measured in disease model supernatants as a surrogate for C3b levels (FIG. 12). Supernatants from RPE cells transduced with the rAAV showed a decreased level of iC3b in comparison to supernatant from non-transduced RPE (FIG. 12). However, as expected, there was no difference in iC3b concentration between non-transduced RPE supernatant and non-transduced RPE supernatant supplemented with eculizumab, since eculizumab regulates the complement system farther downstream in the protein cascade.

In summary, human iPSC-derived RPE cells transduced with rAAV (comprising a capsid protein comprising the amino acid sequence set forth in SEQ ID NO:42 and a heterologous nucleic acid encoding the amino acid sequence set forth in SEQ ID NO:34, operably linked to a CAG promoter) expressed and secreted miniCFH in a dose-dependent manner. The expressed miniCFH exhibited functional alternative complement-inhibiting activity, and specific C3 convertase decay acceleration activity, consistent with the functional activity of endogenous CFH. Furthermore, transduction of RPEs with the rAAV resulted in a protective effect on RPE cultures when the complement pathway was subsequently activated, as assessed by the level of C5b-9/MAC deposition on RPE as well as iC3b levels. MAC is the terminal complex in the complement cascade and a primary effector of complement-mediated cell death. iC3b is a breakdown product of C3b and is a non-endpoint readout of alternative complement activation. Ultimately, soluble iC3b levels were reduced in cultures transduced with the rAAV compared with non-transduced cultures, as was deposition of MAC on RPE as visualized by ICC and quantified by flow cytometric analysis.

### Example 4

In Vivo Ocular Pharmacodynamic Study with rAAV expressing miniCFH in Nonhuman Primates

The safety and miniCFH transgene protein expression of an rAAV (comprising a capsid protein comprising the amino acid sequence set forth in SEQ ID NO:42 and a heterologous nucleic acid encoding the amino acid sequence set forth in SEQ ID NO:34, operably linked to a CAG promoter) in male cynomolgus monkeys following a single intravitreal (IVT) dose to both eyes followed by either a 6 or 12-week observation period was assessed. Male cynomolgus monkeys were dosed once IVT in both eyes with the rAAV in a dose volume of 50 μL/eye. Groups 1-2 consisted of 2 males (group and were dosed with 5×10¹¹ and 5×10¹⁰ vg/eye, respectively and were sacrificed at 6 weeks post-dose for tissue collection. Groups 3, 4 and 5 were dosed once IVT with the rAAV at 5×10¹¹, 5×10¹⁰ and 1×10¹⁰ vg/eye (bilaterally), respectively, and were observed for 12 weeks prior to terminal sacrifice.

Following IVT dosing with the rAAV, samples of aqueous humor, retinal tissue and serum were examined for concentrations of miniCFH protein by LC-MS. A treatment-related dose response for miniCFH protein expression was observed in aqueous humor. (FIG. 13).

miniCFH transgene protein levels were higher in the retina and RPF/choroid, with comparatively low concentrations in aqueous humor (FIG. 14). This pattern of distribution was anticipated given the retinotopic characteristic of the capsid and the fact that retinal tissues are likely a predominant source of miniCFH protein secretion.

miniCFH protein was only detectable in serum from the 5×10¹¹ vg/eye dose groups on Day 15, with very low values close to LLOQ of 5 ng/mL, demonstrating that miniCFH protein expressed from the rAAV is largely contained within the eye following IVT administration and is not distributed to the systemic circulation at meaningful levels (see Table 6 below).

TABLE 6
miniCFH Levels in Aqueous Humor, Vitreous Humor and
Serum in rAAV Treated Cynomolgus Monkeys Samples

| Treatment | Day 15 Serum miniCFH |
|---|---|
| Group | Levels (ng/mL) |
| High dose | 6.07 |
| (5 × 10¹¹ vg/eye) | 0 BQL out of 3 |
| Medium dose | All BQL |
| (5 × 10¹⁰ vg/eye) | |
| Low dose | All BQL |
| (1 × 10¹⁰ vg/eye) | |

BQL: Below quantifiable limit
Lower Limit of Quantification: 5 ng/mL

There were no significant gross macroscopic findings seen in animals sacrificed at Weeks 6 or 12, or any significant changes in clinical pathology, hematology, or coagulation findings across the rAAV dose groups. No treatment-related changes were observed on any clinical pathology parameters following rAAV treatment, and minor fluctuations were generally attributable to the immunosuppression regimen.

Microscopic findings noted following rAAV treatment at 6 weeks included minimal non-adverse perivascular mononuclear cell infiltrates in the retina. Findings at Week 12 were comparable and included minimal to mild non-adverse mononuclear cell infiltrates in the ciliary body, minimal retinal degeneration and pyknotic cells of uncertain significance in one eye, and minimal perivascular mononuclear cell infiltrates in the optic nerve head, none of which were considered adverse. Systemic organs had no microscopic findings in any dose group. IVT treatment with the rAAV resulted in generation of both vector capsid antibodies and anti-miniCFH antibodies in a majority of animals by Week 12. No antigen-specific IFN-γ T-cell responses measured by ELISPOT were detected against either vector capsid or CFH peptide pools in rAAV treated NHP eyes at either 6- or 12-weeks post-dose. Overall, the rAAV was well tolerated following a single bilateral IVT dose in male cynomolgus monkeys at doses up to 5×10¹¹ vg/eye (HED 1×10¹² vg/eye).

### Single Intravitreal Dose Ocular Assessment Study in Cynomolgus Macaques

Another study was initiated to comprehensively assess ocular structure and function parameters in small groups of male and female cynomolgus nonhuman primates following a single unilateral IVT dose of rAAV (comprising a capsid protein comprising the amino acid sequence set forth in SEQ ID NO:42 and a heterologous nucleic acid encoding the amino acid sequence set forth in SEQ ID NO:34, operably linked to a CAG promoter) and follow for 12 weeks post-dose.

Two groups of cynomolgus macaques consisting of 1 male and 2 females per group were dosed once on study Day 1 in the right eye with the rAAV at either 2.8×10¹⁰ vg/eye or 1.5×10¹¹ vg/eye. Animals' left eyes were treated concurrently with vehicle. The dose volume was 50 μL/eye for both right and left eyes.

In-life parameters included daily mortality/cageside assessments, detailed clinical observations once prior to dose during Week-1, pre-dose and at 1, 2, 4 and 8 hrs post-dose on Study Day 1, and on days 2-7, then weekly for the rest of the study. Body weights were assessed prior to dosing, and weekly during the study. Ophthalmic exams were performed pre-test for all animals and on Days 2, 4 (±1), 7, and during Weeks 2, 3, 4, 5, 6, 9 and 12.

Intraocular pressure was assessed pre-study and on Days 2, 4 (I), 7, and during Weeks 2, 3, 4, 5, 6, 9 and 12. Fluorescein angiography, Optical Coherence Tomography (OCT) and wide-field color fundus imaging were evaluated pre-study and during Weeks 3, 6 and 12. Finally, electroretinography (ERG) assessments were performed prior to dosing and during Weeks 6 and 12, prior to terminal sacrifice.

Intravitreal administration of the rAAV at doses of 2.8×10¹⁰ or 1.5×10¹¹ vg/eye was well tolerated in cynomolgus macaques through 12 weeks post-injection as supported by mortality, clinical observation, ophthalmic, clinical pathology, macroscopic pathology, and organ weight parameters. There were no findings of intraocular inflammation at any timepoint, and no rAAV-related changes in IOP values or ocular structure as identified on fluorescein angiography or anterior and posterior segment OCT, and no treatment-related effects on retinal function identified by ERG evaluation.

There were no definitive rAAV treatment-related effects on clinical pathology parameters in either sex following intravitreal administration of the rAAV at 2.8×10¹⁰ or 1.5×10¹¹ vg/eye up to Day 80. Minor fluctuations in hematology and clinical chemistry findings were likely attributable to methylprednisolone administration and were not considered to be related to rAAV treatment.

Overall, the rAAV was well tolerated following a single intravitreal dose in nonhuman primates up to 1.5×10¹¹ vg/eye with no definitive treatment-related effects or changes in ocular structure or function.

While the materials and methods of this invention have been described in terms of preferred embodiments, it will be apparent to those of skill in the art that variations may be applied to the method described herein without departing from the concept, spirit and scope of the invention. All such similar substitutes and modifications apparent to those skilled in the art are deemed to be within the spirit scope and concept of the invention.

1. A recombinant adeno-associated virus (rAAV) comprising (i) a variant AAV capsid protein comprising a heterologous peptide with a length of 7, 8, 9, 10 or 11 amino acids covalently inserted in the GH-loop of the capsid protein relative to a corresponding parental AAV capsid protein, wherein the peptide insertion comprises the amino acid sequence ISDQTKH (SEQ ID NO:1) and (ii) a heterologous nucleic acid comprising a nucleotide sequence encoding a complement regulator factor H (CFH) protein or a fragment thereof.

2. The rAAV according to claim 1, wherein the insertion peptide is LAISDQTKHA (SEQ ID NO:2).

3. The rAAV according to claim 1, wherein the insertion site is located between amino acids corresponding to amino acids 587 and 588 of VP1 of AAV2 (SEQ ID NO:47) or the corresponding position in the capsid protein of another AAV serotype.

4. (canceled)

5. The rAAV according to claim 1, wherein the capsid protein comprises a P34A amino acid substitution relative to VP1 of AAV2 and comprises an amino acid sequence at least 95% identical to the entire length of the amino acid sequence set forth as SEQ ID NO:42, preferably wherein the capsid protein consists of the amino acid sequence set forth as SEQ ID NO:42.

6. (canceled)

7. The rAAV according to claim 1, wherein the variant AAV capsid protein comprises an amino acid sequence with 100% sequence identity to the amino acid sequence set forth in SEQ ID NO:42.

8. The rAAV according to claim 1, wherein the rAAV comprises a heterologous acid comprising from 5′ to 3′: (a) an inverted terminal repeat (b) a promoter (c) a nucleotide sequence encoding the CFH protein or a fragment thereof (d) a polyadenylation sequence and/or a WPRE sequence and (e) an inverted terminal repeat.

9. (canceled)

10. (canceled)

11. The rAAV according to claim 1, wherein the CFH protein fragment consists of SCRs: 1, 2, 3, 4, 19, 20 and one or more of SCR 7, 17 and/or 18, and a leader sequence and one or more linker sequences.

12. The rAAV according to claim 1, wherein the CFH protein fragment consists of SCRs: 1, 2, 3, 4, 19, 20 and one or more of SCR 7, 17 and/or 18 and one or more of SCR5, SCR6, SCR8, SCR16, and a leader sequence and one or more linker sequences.

13. (canceled)

14. The rAAV according to claim 1, wherein the CFH protein fragment consists of a combination of SCR domains selected from one or more of:
(a) SCR1, 2, 3, 4, 7, and 19-20;
(b) SCR1-4, 6, 7, and 19-20;
(c) SCR1-4, 7, 8, and 19-20;
(d) SCR1-4, 6, 7, 8, and 19-20;
(e) SCR1-4, 17, 19-20;
(f) SCR1-4, and 18-20;
(g) SCR1-4, and 17-20;
(h) SCR1-4, 7, and 18-20;
(i) SCR1-4, 6, 7, and 18-20;
(j) SCR1-4, 7, 8, and 18-20;
(k) SCR1-4, 6-8, and 18-20,
(l) SCR1-4, 7, and 17-20;
(m) SCR1-4, 6, 7, and 17-20;
(n) SCR1-4, 7, 8, and 17-20; or
(o) SCR1-4, 6-8, and 17-20,
and a leader sequence and one or more linker sequences.

15. The rAAV according to claim 14, wherein the CFH protein fragment comprises SCR1-4, 6-8, and 17-20 and wherein the CFH protein fragment does not comprise SCR5 and SCR9-16.

16. The rAAV according to claim 15, wherein the CFH protein fragment comprises at least a linker of 1 to about 18 amino acids located between one or more of the SCRs.

17. The rAAV according to claim 16, wherein the CFH protein fragment comprises SCR1-(L1)-SCR2-(L2)-SCR3-(L3)-SCR4-(L4)-(SCR6-(L4′))-SCR7-(L5)-(SCR8-(L5′))-(SCR16-(L5″))-(SCR17-(L5″′))-(SCR1 8-(L5″″))-SCR19-(L6)-SCR20, wherein the ( ) indicate optional component(s), “L” refers to a linker, and each of L1, L2, L3, L4, L4′, L5, L5?, L5″, L5″′, L5″″ and L6 may be absent or independently selected from an amino acid sequence of about 1 to about 12 to about 18 amino acids.

18. (canceled)

19. (canceled)

20. The rAAV according to claim 17, wherein the CFH protein fragment comprises an amino acid sequence with at least 95% sequence identity to the amino acid sequence set forth in SEQ ID NO:34.

21. The rAAV according to claim 17, wherein the CFH protein fragment comprises an amino acid sequence that is 100% identical to the amino acid sequence set forth in SEQ ID NO:34.

22. The rAAV according to claim 17, wherein the CFH protein fragment comprises at least one glycosylation site in one or more of the SCRs.

23. (canceled)

24. The rAAV according to claim 22, wherein the glycosylation site is engineered into one or more of SCR17 and/or SCR18.

25. (canceled)

26. The rAAV according to claim 22, wherein the CFH protein fragment comprises SCR1-4, 6-8, and 17-20 and wherein the glycosylation site is engineered into SCR 17 and SCR18, and wherein the CFH protein fragment does not comprise SCR5 and SCR9-16.

27. The rAAV according to claim 26, wherein the CFH protein fragment comprises an amino acid sequence with 100% sequence identity to the sequence set forth in SEQ ID NO:34 and wherein the glycosylation site is engineered into SCR17 and SCR18.

28. The rAAV according to claim 1, wherein the promoter is a ubiquitous promoter, optionally a CAG promoter.

29. (canceled)

30. (canceled)

31. The rAAV according to claim 28, wherein the rAAV comprises a heterologous nucleic acid comprising a nucleotide sequence with at least 95% sequence identity to the nucleotide sequence set forth in any one of SEQ ID NOs:43-46.

32. (canceled)

33. (canceled)

34. The rAAV according to claim 28, wherein the rAAV comprises a heterologous nucleic acid comprising the nucleotide sequence set forth in any one of SEQ ID NOs:43-46.

35. A host cell comprising the rAAV according to claim 34.

36. A pharmaceutical composition comprising the rAAV according to claim 31 and a pharmaceutically acceptable carrier, diluent, excipient or buffer.

37. A method for treating dry age-related macular disorder (dry AMD) in a subject in need thereof, comprising administering to the subject a pharmaceutical composition according to claim 36 by intravitreal, suprachoroidal or subretinal administration.

38. (canceled)

39. (canceled)

40. A method for treating geographic atrophy (GA) in a subject in need thereof, comprising administering to the subject a therapeutically effective amount of the pharmaceutical composition according to claim 36 by periocular, intravitreal, suprachoroidal or subretinal administration.

41. (canceled)

42. (canceled)

43. (canceled)

44. (canceled)


SEQUENCE LISTING (47 sequences)

SEQ ID NO: 1 - protein, 7 aa, synthetic construct
  REGION 1..7: note = heterologous peptide insertion
ISDQTKH

SEQ ID NO: 2 - protein, 10 aa, synthetic construct
  REGION 1..10: note = heterologous peptide insertion
LAISDQTKHA

SEQ ID NO: 3 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 4 - protein, 18 aa, synthetic construct
  REGION 1..18: note = leader sequence
MRLLAKIICLMLWAICVA

SEQ ID NO: 5 - protein, 62 aa, synthetic construct
  REGION 1..62: note = short complement repeat
EDCNELPPRRNTEILTGSWSDQTYPEGTQAIYKCRPGYRSLGNIIMVCRKGEWVALNPLRKC

SEQ ID NO: 6 - protein, 57 aa, synthetic construct
  REGION 1..57: note = short complement repeat
CGHPGDTPFGTFTLTGGNVFEYGVKAVYTCNEGYQLLGEINYRECDTDGWTNDIPIC

SEQ ID NO: 7 - protein, 60 aa, synthetic construct
  REGION 1..60: note = short complement repeat
CLPVTAPENGKIVSSAMEPDREYHFGQAVRFVCNSGYKIEGDEEMHCSDDGFWSKEKPKC

SEQ ID NO: 8 - protein, 53 aa, synthetic construct
  REGION 1..53: note = short complement repeat
CKSPDVINGSPISQKIIYKENERFQYKCNMGYEYSERGDAVCTESGWRPLPSC

SEQ ID NO: 9 - protein, 54 aa, synthetic construct
  REGION 1..54: note = short complement repeat
CDNPYIPNGDYSPLRIKHRTGDEITYQCRNGFYPATRGNTAKCTSTGWIPAPRC

SEQ ID NO: 10 - protein, 61 aa, synthetic construct
  REGION 1..61: note = short complement repeat
CDYPDIKHGGLYHENMRRPYFPVAVGKYYSYYCDEHFETPSGSYWDHIHCTQDGWSPAVPC

SEQ ID NO: 11 - protein, 54 aa, synthetic construct
  REGION 1..54: note = short complement repeat
CYFPYLENGYNQNYGRKFVQGKSIDVACHPGYALPKAQTTVTCMENGWSPTPRC

SEQ ID NO: 12 - protein, 58 aa, synthetic construct
  REGION 1..58: note = short complement repeat
CSKSSIDIENGFISESQYTYALKEKAKYQCKLGYVTADGETSGSITCGKDGWSAQPTC

SEQ ID NO: 13 - protein, 56 aa, synthetic construct
  REGION 1..56: note = short complement repeat
CDIPVFMNARTKNDFTWFKLNDTLDYECHDGYESNTGSTTGSIVCGYNGWSDLPIC

SEQ ID NO: 14 - protein, 55 aa, synthetic construct
  REGION 1..55: note = short complement repeat
CELPKIDVHLVPDRKKDQYKVGEVLKFSCKPGFTIVGPNSVQCYHFGLSPDLPIC

SEQ ID NO: 15 - protein, 55 aa, synthetic construct
  REGION 1..55: note = short complement repeat
CGPPPELLNGNVKEKTKEEYGHSEVVEYYCNPRFLMKGPNKIQCVDGEWTTLPVC

SEQ ID NO: 16 - protein, 54 aa, synthetic construct
  REGION 1..54: note = short complement repeat
CGDIPELEHGWAQLSSPPYYYGDSVEFNCSESFTMIGHRSITCIHGVWTQLPQC

SEQ ID NO: 17 - protein, 51 aa, synthetic construct
  REGION 1..51: note = short complement repeat
CKSSNLIILEEHLKNKKEFDHNSNIRYRCRGKEGWIHTVCINGRWDPEVNC

SEQ ID NO: 18 - protein, 54 aa, synthetic construct
  REGION 1..54: note = short complement repeat
CPPPPQIPNSHNMTTTLNYRDGEKVSVLCQENYLIQEGEEITCKDGRWQSIPLC

SEQ ID NO: 19 - protein, 57 aa, synthetic construct
  REGION 1..57: note = short complement repeat
CSQPPQIEHGTINSSRSSQESYAHGTKLSYTCEGGFRISEENETTCYMGKWSSPPQC

SEQ ID NO: 20 - protein, 54 aa, synthetic construct
  REGION 1..54: note = short complement repeat
CKSPPEISHGVVAHMSDSYQYGEEVTYKCFEGFGIDGPAIAKCLGEKWSHPPSC

SEQ ID NO: 21 - protein, 55 aa, synthetic construct
  REGION 1..55: note = short complement repeat
CLSLPSFENAIPMGEKKDVYKAGEQVTYTCATYYKMDGASNVTCINSRWTGRPTC

SEQ ID NO: 22 - protein, 55 aa, synthetic construct
  REGION 1..55: note = short complement repeat
CVNPPTVQNAYIVSRQMSKYPSGERVRYQCRSPYEMFGDEEVMCLNGNWTEPPQC

SEQ ID NO: 23 - protein, 55 aa, synthetic construct
  REGION 1..55: note = short complement repeat
CGPPPPIDNGDITSFPLSVYAPASSVEYQCQNLYQLEGNKRITCRNGQWSEPPKC

SEQ ID NO: 24 - protein, 65 aa, synthetic construct
  REGION 1..65: note = short complement repeat
CVISREIMENYNIALRWTAKQKLYSRTGESVEFVCKRGYRLSSRSHTLRTTCWDGKLEYPTCAKR

SEQ ID NO: 25 - protein, 4 aa, synthetic construct
  REGION 1..4: note = linker sequence
QKRP

SEQ ID NO: 26 - protein, 4 aa, synthetic construct
  REGION 1..4: note = linker sequence
EVVK

SEQ ID NO: 27 - protein, 4 aa, synthetic construct
  REGION 1..4: note = linker sequence
VEIS

SEQ ID NO: 28 - protein, 8 aa, synthetic construct
  REGION 1..8: note = linker sequence
EEKSTLKP

SEQ ID NO: 29 - protein, 5 aa, synthetic construct
  REGION 1..5: note = linker sequence
IRVKT

SEQ ID NO: 30 - protein, 4 aa, synthetic construct
  REGION 1..4: note = linker sequence
IKTD

SEQ ID NO: 31 - protein, 9 aa, synthetic construct
  REGION 1..9: note = linker sequence
IKSKDSTGK

SEQ ID NO: 32 - protein, 4 aa, synthetic construct
  REGION 1..4: note = linker sequence
RDTS

SEQ ID NO: 33 - protein, 6 aa, synthetic construct
  REGION 1..6: note = linker sequence
KDSTGK

SEQ ID NO: 34 - protein, 701 aa, synthetic construct
  REGION 1..701: note = factor H variant
MRLLAKIICLMLWAICVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 35 - DNA, 2106 bp, synthetic construct
  misc_feature 1..2106: note = factor H variant coding sequence
  source 1..2106: mol_type = other DNA
atgcgcctgctggccaagatcatctgcctgatgctgtgggccatctgcgtggccgaggactgtaacgagctgccccctcggagaaatacagagatcctgaccggctcttggagcgatcagacctacccagagggcacacaggccatctacaagtgcaggcccggctatcgctccctgggcaatatcatcatggtgtgcaggaagggagagtgggtggccctgaacccactgagaaagtgccagaagaggccatgtggacacccaggcgacacacctttcggcacctttacactgaccggcggcaacgtgttcgagtacggcgtgaaggccgtgtatacctgcaatgagggctaccagctgctgggcgagatcaactaccgggagtgtgacacagatggctggaccaatgatatccccatctgcgaggtggtgaagtgtctgccagtgaccgcccccgagaacggcaagatcgtgagctccgccatggagcctgacagagagtatcacttcggccaggccgtgaggttcgtgtgcaatagcggctacaagatcgagggcgatgaggagatgcactgttccgacgatggcttctggtctaaggagaagcccaagtgcgtggagatctcctgtaagtctcctgacgtgattaacggcagcccaatctcccagaagatcatctataaggagaatgagcggtttcagtacaagtgcaacatgggctacgagtattccgagagaggcgatgccgtgtgcacagagtctggatggaggcccctgcctagctgcgaggagaagtccaccctgaagccttgtgactatccagatatcaagcacggcggcctgtatcacgagaatatgaggcgcccttacttcccagtggccgtgggcaagtactattcctactattgcgacgagcactttgagacaccatccggctcttactgggaccacatccactgtacccaggatggctggtctcccgccgtgccttgcctgagaaagtgttacttcccctatctggagaacggctacaaccagaattatggcaggaagtttgtgcagggcaagtccatcgacgtggcatgccacccaggatacgcactgcctaaggcacagaccacagtgacctgtatggagaatggctggagcccaacacccaggtgcatccgcgtgaagacctgttccaagagcagcatcgacatcgagaacggcttcatcagcgagtcccagtacacctatgccctgaaggagaaggccaagtatcagtgcaagctgggatacgtgaccgcagacggagaaacatctggcagcatcacctgcggcaaggatggctggtctgcccagcccacatgcatcaagagcatcaagaccgactgtctgtctctgcccagctttgagaatgccatccctatgggcgagaagaaggacgtgtacaaggccggcgagcaggtgacatacacctgcgccacatactataagatggacggcgccagcaacgtgacctgtatcaattcccggtggacaggcagacctacctgcagggatacaagctgcgtgaacccacccaccgtgcagaatgcctatatcgtgtcccgccagatgtctaagtaccctagcggcgagcgggtgagatatcagtgccggtccccatacgagatgttcggcgacgaggaagtgatgtgcctgaacggcaattggacagagcctccacagtgcaaggatagcaccggcaagtgtggaccacctccaccaatcgacaacggcgatatcacatcctttccactgagcgtgtacgcccccgcctcctctgtggagtatcagtgccagaacctgtaccagctggagggcaataagcgcatcacctgtcggaacggccagtggtctgagcctccaaagtgcctgcacccttgcgtgatctccagagagatcatggagaactataatatcgccctgcgctggacagccaagcagaagctgtactctcggaccggcgagagcgtggagttcgtgtgcaagcggggctatagactgagctcccgcagccacacactgcggaccacatgctgggacggcaagctggagtacccaacctgtgccaagaggtga

SEQ ID NO: 36 - DNA, 2106 bp, synthetic construct
  misc_feature 1..2106: note = factor H variant coding sequence
  source 1..2106: mol_type = other DNA
atgaggctgctggccaagatcatctgcctgatgctgtgggccatctgtgtggctgaggactgtaatgagctgccccctaggagaaatacagagatcctgactggctcttggtctgatcagacctacccagagggcacacaggccatctacaagtgcaggcctggctatagatccctgggcaatatcatcatggtgtgcaggaagggagagtgggtggccctgaacccactgagaaagtgccagaagaggccatgtggacacccaggagacacaccttttggcacctttacactgacagggggcaatgtgtttgagtatggtgtgaaggcagtgtatacctgcaatgagggctaccagctgctgggagagatcaactacagggagtgtgacacagatggctggaccaatgatatccccatctgtgaggtggtgaagtgtctgccagtgactgcccctgagaatggcaagatagtgagctctgccatggagcctgacagagagtatcactttggccaggctgtgaggtttgtgtgcaatagtggctacaagattgagggggatgaggagatgcactgttcagatgatggcttctggtctaaggagaagcccaagtgtgtggagatctcctgtaagtctcctgatgtgattaatggcagcccaatctcccagaagatcatctataaggagaatgagagatttcagtacaagtgcaacatgggctatgagtattcagagagaggtgatgctgtgtgcacagagtctggatggaggcccctgcctagctgtgaggagaagtccaccctgaagccttgtgactatccagatatcaagcatggaggcctgtatcatgagaatatgaggaggccttacttcccagtggctgtgggcaagtactattcctactattgtgatgagcactttgagacaccatctggctcttactgggaccacatccactgtacccaggatggctggtctcctgctgtgccttgcctgagaaagtgttacttcccctatctggagaatggctacaaccagaattatggcaggaagtttgtgcagggcaagtccattgatgtggcatgccacccaggatatgcactgcctaaggcacagaccacagtgacctgtatggagaatggctggagcccaacacccaggtgcatcagggtgaagacctgttccaagagcagcattgacatagagaatggcttcatctcagagtcccagtacacctatgccctgaaggagaaggccaagtatcagtgcaagctgggatatgtgacagcagatggagaaacatctggcagcatcacctgtggcaaggatggctggtctgcccagcccacatgcatcaagagcatcaagactgactgtctgtctctgcccagctttgagaatgccatccctatgggtgagaagaaggatgtgtacaaggctggagagcaggtgacatacacctgtgccacatactataagatggatggagccagcaatgtgacctgtatcaattccagatggacaggcagacctacctgcagggatacaagctgtgtgaacccacccactgtgcagaatgcctatattgtgtccaggcagatgtctaagtacccttctggagagagagtgagatatcagtgcagatccccatatgagatgtttggggatgaggaagtgatgtgcctgaatggcaattggacagagcctccacagtgcaaggatagcactggcaagtgtggaccacctccaccaattgacaatggagatatcacatcctttccactgtcagtgtatgcccctgcctcctctgtggagtatcagtgccagaacctgtaccagctggagggcaataagagaatcacctgtagaaatggccagtggtctgagcctccaaagtgcctgcacccttgtgtgatctccagagagatcatggagaactataatatagccctgaggtggacagccaagcagaagctgtactctaggactggggagtcagtggagtttgtgtgcaagagaggctatagactgagctccagaagccacacactgaggaccacatgctgggatggcaagctggagtacccaacctgtgccaagaggtga

SEQ ID NO: 37 - DNA, 2106 bp, synthetic construct
  misc_feature 1..2106: note = factor H variant coding sequence
  source 1..2106: mol_type = other DNA
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgcaggcctggatatagatctcttggaaatatcataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattacagggaatgtgacacagatggatggaccaatgatattcctatatgtgaagttgtgaagtgtttaccagtgacagcaccagagaatggaaaaattgtcagtagtgcaatggaaccagatagagaataccattttggacaagcagtaagatttgtatgtaactcaggctacaagattgaaggagatgaagaaatgcattgttcagatgatggtttttggagtaaagagaaaccaaagtgtgtggaaatttcatgcaaatccccagatgttataaatggatctcctatatctcagaagattatttataaggagaatgaaagatttcaatataaatgtaacatgggttatgaatacagtgaaagaggagatgctgtatgcactgaatctggatggagacctttgccttcatgtgaagaaaaatcaaccttgaaaccttgtgattatccagacattaaacatggaggtctatatcatgagaatatgagaagaccatactttccagtagctgtaggaaaatattactcctattactgtgatgaacattttgagactccttcaggaagttactgggatcacattcattgcacacaagatggatggagtccagcagtaccatgcctcagaaaatgttattttccttatttggaaaatggatataatcaaaattatggaagaaagtttgtacagggtaaatctatagatgttgcctgccatcctggctatgctcttccaaaagctcagaccacagttacatgtatggagaatggctggtctcctactcccagatgcatcagagtcaaaacatgttccaaatcaagtatagatattgagaatgggtttatttctgaatctcagtatacatatgccttaaaagaaaaagctaaatatcaatgcaaactaggatatgtaacagcagatggtgaaacatcaggatcaattacatgtgggaaagatggatggtcagctcaacccacctgcattaaatctataaaaacagattgtctcagtttacctagctttgaaaatgccatacccatgggagagaagaaggatgtgtataaggctggtgagcaagtgacttacacttgtgcaacatattacaaaatggatggagccagtaatgtaacatgcattaatagcagatggacaggaaggccaacatgcagagacacctcctgtgtgaatcctcccacagtacaaaatgcttatatagtgagtagacagatgagtaaatatccatctggtgagagagtaagatatcaatgtaggagcccttatgaaatgtttggggatgaagaagtgatgtgtttaaatggaaactggacagaaccacctcaatgcaaagattctacaggaaaatgtgggccccctccacctattgacaatggggacattacttcattccccttgtcagtatatgctccagcttcatcagttgagtaccaatgccagaacttgtatcaacttgagggtaacaagaggataacatgtagaaatggacaatggtcagaaccaccaaaatgcttacatccctgtgtaatatccagggaaattatggaaaattataacatagcattaaggtggacagccaaacagaagctttattctagaacaggtgaatcagttgaatttgtgtgtaaaagaggatatagactttcatcaaggtctcacacattgaggacaacatgttgggatgggaaactggagtatccaacttgtgcaaaaagatga

SEQ ID NO: 38 - DNA, 145 bp, synthetic construct
  misc_feature 1..145: note = 5' ITR
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 39 - DNA, 145 bp, synthetic construct
  misc_feature 1..145: note = 3' ITR
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 40 - DNA, 1664 bp, synthetic construct
  misc_feature 1..1664: note = CAG promoter
  source 1..1664: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 41 - DNA, 245 bp, synthetic construct
  misc_feature 1..245: note = SV40 polyadenlyation
  source 1..245: mol_type = other DNA
ggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatca

SEQ ID NO: 42 - protein, 745 aa, synthetic construct
  REGION 1..745: note = variant Adeno-Associated Virus capsid
MAADGYLPDWLEDTLSEGIRQWWKLKPGPPPPKAAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAISDQTKHARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 43 - DNA, 4432 bp, synthetic construct
  misc_feature 1..4432: note = heterologous nucleic acid sequence
  source 1..4432: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaattccccggggatccactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacctgcaggtggatatcttgctagcacgccaccatgcgcctgctggccaagatcatctgcctgatgctgtgggccatctgcgtggccgaggactgtaacgagctgccccctcggagaaatacagagatcctgaccggctcttggagcgatcagacctacccagagggcacacaggccatctacaagtgcaggcccggctatcgctccctgggcaatatcatcatggtgtgcaggaagggagagtgggtggccctgaacccactgagaaagtgccagaagaggccatgtggacacccaggcgacacacctttcggcacctttacactgaccggcggcaacgtgttcgagtacggcgtgaaggccgtgtatacctgcaatgagggctaccagctgctgggcgagatcaactaccgggagtgtgacacagatggctggaccaatgatatccccatctgcgaggtggtgaagtgtctgccagtgaccgcccccgagaacggcaagatcgtgagctccgccatggagcctgacagagagtatcacttcggccaggccgtgaggttcgtgtgcaatagcggctacaagatcgagggcgatgaggagatgcactgttccgacgatggcttctggtctaaggagaagcccaagtgcgtggagatctcctgtaagtctcctgacgtgattaacggcagcccaatctcccagaagatcatctataaggagaatgagcggtttcagtacaagtgcaacatgggctacgagtattccgagagaggcgatgccgtgtgcacagagtctggatggaggcccctgcctagctgcgaggagaagtccaccctgaagccttgtgactatccagatatcaagcacggcggcctgtatcacgagaatatgaggcgcccttacttcccagtggccgtgggcaagtactattcctactattgcgacgagcactttgagacaccatccggctcttactgggaccacatccactgtacccaggatggctggtctcccgccgtgccttgcctgagaaagtgttacttcccctatctggagaacggctacaaccagaattatggcaggaagtttgtgcagggcaagtccatcgacgtggcatgccacccaggatacgcactgcctaaggcacagaccacagtgacctgtatggagaatggctggagcccaacacccaggtgcatccgcgtgaagacctgttccaagagcagcatcgacatcgagaacggcttcatcagcgagtcccagtacacctatgccctgaaggagaaggccaagtatcagtgcaagctgggatacgtgaccgcagacggagaaacatctggcagcatcacctgcggcaaggatggctggtctgcccagcccacatgcatcaagagcatcaagaccgactgtctgtctctgcccagctttgagaatgccatccctatgggcgagaagaaggacgtgtacaaggccggcgagcaggtgacatacacctgcgccacatactataagatggacggcgccagcaacgtgacctgtatcaattcccggtggacaggcagacctacctgcagggatacaagctgcgtgaacccacccaccgtgcagaatgcctatatcgtgtcccgccagatgtctaagtaccctagcggcgagcgggtgagatatcagtgccggtccccatacgagatgttcggcgacgaggaagtgatgtgcctgaacggcaattggacagagcctccacagtgcaaggatagcaccggcaagtgtggaccacctccaccaatcgacaacggcgatatcacatcctttccactgagcgtgtacgcccccgcctcctctgtggagtatcagtgccagaacctgtaccagctggagggcaataagcgcatcacctgtcggaacggccagtggtctgagcctccaaagtgcctgcacccttgcgtgatctccagagagatcatggagaactataatatcgccctgcgctggacagccaagcagaagctgtactctcggaccggcgagagcgtggagttcgtgtgcaagcggggctatagactgagctcccgcagccacacactgcggaccacatgctgggacggcaagctggagtacccaacctgtgccaagaggtgagccacgcgtaacacgtgcatgcgagagatctgcggccgcgagctcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatcctagccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 44 - DNA, 4696 bp, synthetic construct
  misc_feature 1..4696: note = heterologous nucleic acid sequence
  source 1..4696: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaaccaggcgcgcccagtttaaacagtccggaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacggtcaccggtcagctagcacgccaccatgcgcctgctggccaagatcatctgcctgatgctgtgggccatctgcgtggccgaggactgtaacgagctgccccctcggagaaatacagagatcctgaccggctcttggagcgatcagacctacccagagggcacacaggccatctacaagtgcaggcccggctatcgctccctgggcaatatcatcatggtgtgcaggaagggagagtgggtggccctgaacccactgagaaagtgccagaagaggccatgtggacacccaggcgacacacctttcggcacctttacactgaccggcggcaacgtgttcgagtacggcgtgaaggccgtgtatacctgcaatgagggctaccagctgctgggcgagatcaactaccgggagtgtgacacagatggctggaccaatgatatccccatctgcgaggtggtgaagtgtctgccagtgaccgcccccgagaacggcaagatcgtgagctccgccatggagcctgacagagagtatcacttcggccaggccgtgaggttcgtgtgcaatagcggctacaagatcgagggcgatgaggagatgcactgttccgacgatggcttctggtctaaggagaagcccaagtgcgtggagatctcctgtaagtctcctgacgtgattaacggcagcccaatctcccagaagatcatctataaggagaatgagcggtttcagtacaagtgcaacatgggctacgagtattccgagagaggcgatgccgtgtgcacagagtctggatggaggcccctgcctagctgcgaggagaagtccaccctgaagccttgtgactatccagatatcaagcacggcggcctgtatcacgagaatatgaggcgcccttacttcccagtggccgtgggcaagtactattcctactattgcgacgagcactttgagacaccatccggctcttactgggaccacatccactgtacccaggatggctggtctcccgccgtgccttgcctgagaaagtgttacttcccctatctggagaacggctacaaccagaattatggcaggaagtttgtgcagggcaagtccatcgacgtggcatgccacccaggatacgcactgcctaaggcacagaccacagtgacctgtatggagaatggctggagcccaacacccaggtgcatccgcgtgaagacctgttccaagagcagcatcgacatcgagaacggcttcatcagcgagtcccagtacacctatgccctgaaggagaaggccaagtatcagtgcaagctgggatacgtgaccgcagacggagaaacatctggcagcatcacctgcggcaaggatggctggtctgcccagcccacatgcatcaagagcatcaagaccgactgtctgtctctgcccagctttgagaatgccatccctatgggcgagaagaaggacgtgtacaaggccggcgagcaggtgacatacacctgcgccacatactataagatggacggcgccagcaacgtgacctgtatcaattcccggtggacaggcagacctacctgcagggatacaagctgcgtgaacccacccaccgtgcagaatgcctatatcgtgtcccgccagatgtctaagtaccctagcggcgagcgggtgagatatcagtgccggtccccatacgagatgttcggcgacgaggaagtgatgtgcctgaacggcaattggacagagcctccacagtgcaaggatagcaccggcaagtgtggaccacctccaccaatcgacaacggcgatatcacatcctttccactgagcgtgtacgcccccgcctcctctgtggagtatcagtgccagaacctgtaccagctggagggcaataagcgcatcacctgtcggaacggccagtggtctgagcctccaaagtgcctgcacccttgcgtgatctccagagagatcatggagaactataatatcgccctgcgctggacagccaagcagaagctgtactctcggaccggcgagagcgtggagttcgtgtgcaagcggggctatagactgagctcccgcagccacacactgcggaccacatgctgggacggcaagctggagtacccaacctgtgccaagaggtgagcctaggactgtacagtacgcgtaacacgtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatggtcccagccttcattgagcctattggtcccccctcagaacccacccacctggcagtagaggatgtctcttgagacaccactgtctccctcaagttcaggaggcccccattaattaagagagagtgggagcaggaggcctgtcagatggctactcttaagtggagtactgcccagagggctgctcagagtgggtggctgtaaccctgcaggggctgacagagcacacatccatactggtgaaggacctgtgacccactggccagactggccggccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 45 - DNA, 4696 bp, synthetic construct
  misc_feature 1..4696: note = heterologous nucleic acid sequence
  source 1..4696: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaaccaggcgcgcccagtttaaacagtccggaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacggtcaccggtcagctagcacgccaccatgaggctgctggccaagatcatctgcctgatgctgtgggccatctgtgtggctgaggactgtaatgagctgccccctaggagaaatacagagatcctgactggctcttggtctgatcagacctacccagagggcacacaggccatctacaagtgcaggcctggctatagatccctgggcaatatcatcatggtgtgcaggaagggagagtgggtggccctgaacccactgagaaagtgccagaagaggccatgtggacacccaggagacacaccttttggcacctttacactgacagggggcaatgtgtttgagtatggtgtgaaggcagtgtatacctgcaatgagggctaccagctgctgggagagatcaactacagggagtgtgacacagatggctggaccaatgatatccccatctgtgaggtggtgaagtgtctgccagtgactgcccctgagaatggcaagatagtgagctctgccatggagcctgacagagagtatcactttggccaggctgtgaggtttgtgtgcaatagtggctacaagattgagggggatgaggagatgcactgttcagatgatggcttctggtctaaggagaagcccaagtgtgtggagatctcctgtaagtctcctgatgtgattaatggcagcccaatctcccagaagatcatctataaggagaatgagagatttcagtacaagtgcaacatgggctatgagtattcagagagaggtgatgctgtgtgcacagagtctggatggaggcccctgcctagctgtgaggagaagtccaccctgaagccttgtgactatccagatatcaagcatggaggcctgtatcatgagaatatgaggaggccttacttcccagtggctgtgggcaagtactattcctactattgtgatgagcactttgagacaccatctggctcttactgggaccacatccactgtacccaggatggctggtctcctgctgtgccttgcctgagaaagtgttacttcccctatctggagaatggctacaaccagaattatggcaggaagtttgtgcagggcaagtccattgatgtggcatgccacccaggatatgcactgcctaaggcacagaccacagtgacctgtatggagaatggctggagcccaacacccaggtgcatcagggtgaagacctgttccaagagcagcattgacatagagaatggcttcatctcagagtcccagtacacctatgccctgaaggagaaggccaagtatcagtgcaagctgggatatgtgacagcagatggagaaacatctggcagcatcacctgtggcaaggatggctggtctgcccagcccacatgcatcaagagcatcaagactgactgtctgtctctgcccagctttgagaatgccatccctatgggtgagaagaaggatgtgtacaaggctggagagcaggtgacatacacctgtgccacatactataagatggatggagccagcaatgtgacctgtatcaattccagatggacaggcagacctacctgcagggatacaagctgtgtgaacccacccactgtgcagaatgcctatattgtgtccaggcagatgtctaagtacccttctggagagagagtgagatatcagtgcagatccccatatgagatgtttggggatgaggaagtgatgtgcctgaatggcaattggacagagcctccacagtgcaaggatagcactggcaagtgtggaccacctccaccaattgacaatggagatatcacatcctttccactgtcagtgtatgcccctgcctcctctgtggagtatcagtgccagaacctgtaccagctggagggcaataagagaatcacctgtagaaatggccagtggtctgagcctccaaagtgcctgcacccttgtgtgatctccagagagatcatggagaactataatatagccctgaggtggacagccaagcagaagctgtactctaggactggggagtcagtggagtttgtgtgcaagagaggctatagactgagctccagaagccacacactgaggaccacatgctgggatggcaagctggagtacccaacctgtgccaagaggtgagcctaggactgtacagtacgcgtaacacgtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatggtcccagccttcattgagcctattggtcccccctcagaacccacccacctggcagtagaggatgtctcttgagacaccactgtctccctcaagttcaggaggcccccattaattaagagagagtgggagcaggaggcctgtcagatggctactcttaagtggagtactgcccagagggctgctcagagtgggtggctgtaaccctgcaggggctgacagagcacacatccatactggtgaaggacctgtgacccactggccagactggccggccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 46 - DNA, 4696 bp, synthetic construct
  misc_feature 1..4696: note = heterologous nucleic acid sequence
  source 1..4696: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaaccaggcgcgcccagtttaaacagtccggaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacggtcaccggtcagctagcacgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgcaggcctggatatagatctcttggaaatatcataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattacagggaatgtgacacagatggatggaccaatgatattcctatatgtgaagttgtgaagtgtttaccagtgacagcaccagagaatggaaaaattgtcagtagtgcaatggaaccagatagagaataccattttggacaagcagtaagatttgtatgtaactcaggctacaagattgaaggagatgaagaaatgcattgttcagatgatggtttttggagtaaagagaaaccaaagtgtgtggaaatttcatgcaaatccccagatgttataaatggatctcctatatctcagaagattatttataaggagaatgaaagatttcaatataaatgtaacatgggttatgaatacagtgaaagaggagatgctgtatgcactgaatctggatggagacctttgccttcatgtgaagaaaaatcaaccttgaaaccttgtgattatccagacattaaacatggaggtctatatcatgagaatatgagaagaccatactttccagtagctgtaggaaaatattactcctattactgtgatgaacattttgagactccttcaggaagttactgggatcacattcattgcacacaagatggatggagtccagcagtaccatgcctcagaaaatgttattttccttatttggaaaatggatataatcaaaattatggaagaaagtttgtacagggtaaatctatagatgttgcctgccatcctggctatgctcttccaaaagctcagaccacagttacatgtatggagaatggctggtctcctactcccagatgcatcagagtcaaaacatgttccaaatcaagtatagatattgagaatgggtttatttctgaatctcagtatacatatgccttaaaagaaaaagctaaatatcaatgcaaactaggatatgtaacagcagatggtgaaacatcaggatcaattacatgtgggaaagatggatggtcagctcaacccacctgcattaaatctataaaaacagattgtctcagtttacctagctttgaaaatgccatacccatgggagagaagaaggatgtgtataaggctggtgagcaagtgacttacacttgtgcaacatattacaaaatggatggagccagtaatgtaacatgcattaatagcagatggacaggaaggccaacatgcagagacacctcctgtgtgaatcctcccacagtacaaaatgcttatatagtgagtagacagatgagtaaatatccatctggtgagagagtaagatatcaatgtaggagcccttatgaaatgtttggggatgaagaagtgatgtgtttaaatggaaactggacagaaccacctcaatgcaaagattctacaggaaaatgtgggccccctccacctattgacaatggggacattacttcattccccttgtcagtatatgctccagcttcatcagttgagtaccaatgccagaacttgtatcaacttgagggtaacaagaggataacatgtagaaatggacaatggtcagaaccaccaaaatgcttacatccctgtgtaatatccagggaaattatggaaaattataacatagcattaaggtggacagccaaacagaagctttattctagaacaggtgaatcagttgaatttgtgtgtaaaagaggatatagactttcatcaaggtctcacacattgaggacaacatgttgggatgggaaactggagtatccaacttgtgcaaaaagatgagcctaggactgtacagtacgcgtaacacgtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatggtcccagccttcattgagcctattggtcccccctcagaacccacccacctggcagtagaggatgtctcttgagacaccactgtctccctcaagttcaggaggcccccattaattaagagagagtgggagcaggaggcctgtcagatggctactcttaagtggagtactgcccagagggctgctcagagtgggtggctgtaaccctgcaggggctgacagagcacacatccatactggtgaaggacctgtgacccactggccagactggccggccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 47 - protein, 735 aa, adeno-associated virus 2 [sequence omitted]